(19) United States
(12) Patent Application Publication
(10) Pub. No.: US 2006/0265769 A1
(43) Pub. Date: Nov. 23, 2006

(54) Gab2 (p97) gene and methods of use thereof

(75) Inventors: Haihua Gu, Needham, MA (US); Benjamin G. Neel, Wayland, MA (US); Jean-Pierre Kinet, Lexington, MA (US)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: Beth Israel Deaconess Medical Center, Inc.

(21) Appl. No.: 11/300,682

(22) Filed: Dec. 13, 2005

Related U.S. Application Data
(63) Continuation-in-part of application No. 11/155,004, filed on Jun. 15, 2005.
Application No. 11/155,004 is a continuation of application No. 10/424,570, filed on Apr. 25, 2003, now abandoned.
Application No. 10/424,570 is a continuation of application No. PCT/US01/47854, filed on Oct. 26, 2001.
(60) Provisional application No. 60/243,495, filed on Oct. 26, 2000.

(51) Int. Cl. A01K 67/027; C12Q 1/68; C07H 21/04; C12P 21/06; C07K 14/74

(52) U.S. Cl. 800/18; 435/69.1; 435/320.1; 435/325; 530/350; 514/12; 435/6; 536/23.5

(57) ABSTRACT

This invention relates to the purification, cloning and characterization of a novel gene, Gab2. In response to extracellular stimuli (e.g., cyokines, growth factors, hormones and antigens), Gab2 binds several signal relay molecules, including the protein-tyrosine phosphatase SHP-2 and phosphatidylinositol-3-OH kinase (PI-3K), which results in the initiation of multiple signaling cascades. Gab2 nucleic acid molecules, peptides, vectors, host cells, probes, antibodies, knockout and transgenic animals are provided. The invention also relates to methods of diagnosis, prevention and treatment of Gab2-mediated conditions such as allergic responses, neoplastic disorders and immune disorders. The invention further relates to diagnostic kits for disorders associated with altered Gab2 expression.

### RELATED APPLICATIONS

This application is a continuation-in-part of U.S. application Ser. No. 11/155,004, filed Jun. 15, 2005, which is a continuation of the U.S. application Ser. No. 10/424,570, which was filed on Apr. 25, 2003, which is a continuation of International Application No. PCT/US01/47854, which designated the United States and was filed Oct. 26, 2001, published in English, and which claimed the benefit of U.S. Provisional Application No. 60/243,495, filed Oct. 26, 2000. The entire teachings of the above Applications are incorporated herein by reference.

### GOVERNMENT SUPPORT

The invention was supported, in whole or in part, by grants R01 DK50693, P01 DK50654, R01 DK50654, R01 CA49152 and R01 AI 51612 from the National Institutes of Health and grant DAMD170310284 from the Department of Defense. The Government has certain rights in the invention.

### BACKGROUND OF THE INVENTION

Extracellular stimuli are involved in a number of biological processes including cellular proliferation and differentiation. Several components in such biological processes (e.g., signaling cascades) remain unidentified. Thus, there is a need to identify new components of signaling cascades as well as to develop new, improved and effective methods to identify components of signaling cascades.

Allergies are a major medical problem with significant morbidity and even occasional mortality. Current anti-allergy drugs include primarily anti-histamines, cromolyn sodium, and steroids. Steroids can have undesirable side effects. Cromolyn sodium is mainly useful for chronic prevention and often is an irritant in its own right. Antihistamines block the effects of mast cell degranulation, but do not prevent degranulation itself. There is a large market for new anti-allergy agents, as evinced by the success of non-sedating antihistamines when they were introduced several years ago.

Cancer progression is a multi-step process, which evolves from the accumulation of mutations and the deregulation of the genes involved in cell-growth control. In the case of human breast cancer, commonly observed genetic abnormalities include the loss of heterozygosity (LOH) in tumor suppressor genes and the DNA amplification and/or overexpression of growth promoting oncogenes (e.g., c-myc, cyclin D1, and ErbB2/Neu). Breast carcinogenesis due to amplification of ErbB2, in association with Gab2 overexpression resulting from Gab2 amplification, may provide not only a new diagnostic marker for breast cancer detection but also an alternative therapeutic strategy for treating some breast tumors. Since Gab2 knockout mice are essentially healthy except for defects in allergic response, intervention to specifically lower the expression of Gab2 in vivo should have minimal side effects.

### SUMMARY OF THE INVENTION

The invention relates to the isolation, cloning, sequencing and characterization of the Gab2 gene or a fragment, derivative or mutation thereof. The present invention also relates to DNA molecules (also referred to herein as DNA sequences or nucleic acid sequences) which encode a protein which comprises Gab2. The present invention also relates to DNA sequences capable of hybridizing to the DNA sequence of Gab2.

The present invention further relates to an expression vector comprising the nucleic acid molecule of Gab2, or a fragment, derivative or mutation thereof. The present invention also relates to a host cell which has been transformed, or transfected, with the expression vector comprising the nucleic acid molecule of Gab2, or a fragment, derivative or mutation thereof.

The protein, or peptides, of the present invention can be used to produce antibodies, both polyclonal and monoclonal, which are reactive with (i.e., bind to) the Gab2 protein, and can be used in diagnostic assays to determine the presence or type of a Gab2-mediated, e.g., Gab2-dependent, disease.

The invention further relates to a transgenic non-human mammal (e.g., a mouse) with a disruption of the Gab2 gene in its genome, either a homozygous disruption or a heterozygous disruption, such that the mammal lacks, or has reduced levels of, functional Gab2 protein. The invention also relates to a transgenic non-human mammal (e.g., a mouse) in which the genome has been altered such that the mammal has increased levels of functional Gab2 protein. These transgenic mammals would exhibit an altered responsiveness to cytokine, growth factor, hormone or antigen stimulation.

The invention further relates to the use of an agent which inhibits a Gab2 interaction with an associated protein (e.g., SHP-2, p85, PI-3K, Grb2, ERBB2/HER2/Neu, an ERBB2/HER2/Neu oncogenic mutant protein), in response to an extracellular stimulus (e.g., a cytokine, growth factor, hormone or antigen) for the manufacture of a medicament for preventing or treating a Gab2-mediated injury (e.g., an allergic response, a neoplastic disease, or an immune disorder).

The agent of the present invention can be selected from the group consisting of proteins, polypeptides, antibodies, oligonucleotides, small molecules, natural product inhibitors, mutants of Gab2 (e.g., a mutant Gab2 protein that is unable to bind to SHP-2 protein), and mutants of Gab2-associated molecules, or an agent which is an oligonucleotide antisense to the nucleic acid sequence of Gab2 (e.g., a microRNA that targets Gab2), or antisense to a Gab2 homolog, fragment, complementary sequence, or mutant.

The invention further relates to the nasal, topical or systemic administration of the agent in which the agent is a mutant Gab2, or fragment thereof, which competes with Gab2 for interaction with its associated proteins, or the agent inhibits the expression of Gab2 or the agent inhibits the tyrosyl phosphorylation of Gab2. The invention also relates to the administration of the agent as an insert in a gene therapy vector.

The agent of the present invention can also be employed to inhibit the response of mast cells to FceRI receptor stimulation by administration of the agent to the mast cells. In particular, the agent can prevent a Gab2-mediated injury, for example, an allergic response, by inhibiting a Gab2 interaction with an associated protein in response to allergen challenge and, thus, prevent activation of a Gab2-mediated signaling cascade (e.g., an PI3K signaling pathway).

The agent of the present invention can also be employed to inhibit a neoplastic disease (e.g., leukemia, prostate cancer, ovarian cancer and breast cancer) by inhibiting a Gab2 interaction with an associated protein in response to an extracellular signal and, thus, prevent activation of a Gab2-mediated signaling cascade. In a particular embodiment, the neoplastic disease is a breast cancer associated with Gab2 overexpression. As used herein, the phrase “is associated with” means “is characterized by” or “is correlated with” one or more particular features. In a related embodiment, the neoplastic disease is a breast cancer associated with a Gab2 gene amplification. In yet a further embodiment, the neoplastic disease is a breast cancer associated with ERBB2/HER2/Neu overexpression or a breast cancer associated with the expression of a ERBB2/HER2/Neu oncogenic mutant protein. The Gab2 nucleic acid sequence and Gab2 amino acid sequence of the present invention can be used to produce a probe and antibody, respectively, that can detect upregulation of Gab2 product in a patient with a neoplastic disorder.

The invention also relates to a method of identifying a drug to that can be administered to treat a Gab2-mediated condition by producing a mouse that is a model of the condition, and administering to the mouse a drug to be assessed for its effectiveness in treating or preventing the condition. If the drug reduces the extent to which the condition is present or progresses, the drug is a drug to be administered to treat the condition.

The present invention also relates to isolated RNA molecules (double-stranded; single-stranded) which mediate RNAi of Gab2. That is, the isolated RNAs of the present invention mediate degradation of Gab2 mRNA. It is not necessary that there be perfect correspondence of the sequences, but the correspondence must be sufficient to enable the RNA to direct RNAi cleavage of the Gab2 mRNA. In a particular embodiment, the RNA molecules of the present invention comprise a 3′ hydroxyl group.

The present invention also relates to RNA produced by the methods of the present invention, as well as to RNAs, produced by other methods, such as chemical synthesis or recombinant DNA techniques, that have the same or substantially the same sequences as naturally-occurring RNAs that mediate Gab2 RNAi, such as those produced by the methods of the present invention. The invention further relates to uses of the RNAs, such as for therapeutic or prophylactic treatment and compositions comprising RNAs that mediate Gab2 RNAi, such as pharmaceutical compositions comprising RNAs and an appropriate carrier (e.g., a buffer or water).

The present invention also relates to a method of mediating RNA interference of Gab2 mRNA of a gene in a cell or organism (e.g., mammal such as a mouse or a human). In one embodiment, RNA which targets the Gab2 mRNA is introduced into the cell or organism. The cell or organism is maintained under conditions under which degradation of the mRNA occurs, thereby mediating RNA interference of the Gab2 mRNA in the cell or organism. As used herein, the term “cell or organism in which RNAi occurs” includes both a cell or organism in which Gab2 RNAi occurs as the cell or organism is obtained, or a cell or organism that has been modified so that RNAi occurs.

The present invention also relates to a method for knocking down (partially or completely) the Gab2 gene, thus providing an alternative to presently available methods of knocking down (or out) the Gab2 gene. This method of knocking down gene expression can be used therapeutically or for research (e.g., to generate models of disease states, to examine the function of a gene, to assess whether an agent acts on a gene, to validate targets for drug discovery). In those instances in which gene function is eliminated, the resulting cell or organism can also be referred to as a knockout. One embodiment of the method of producing knockdown cells and organisms comprises introducing into a cell or organism in which Gab2 is to be knocked down, RNA that targets the Gab2 gene and maintaining the resulting cell or organism under conditions under which RNAi occurs, resulting in degradation of the Gab2 mRNA, thereby producing knockdown cells or organisms. Knockdown cells and organisms produced by the present method are also the subject of this invention.

The present invention also encompasses a method of treating a disease or condition associated with the presence of the Gab2 protein in an individual comprising administering to the individual RNA which targets the Gab2 mRNA for degradation. As a result, the protein is not produced or is not produced to the extent it would be in the absence of the treatment.

Also encompassed by the present invention is a method of identifying target sites within a Gab2 mRNA that are particularly suitable for RNAi as well as a method of assessing the ability of RNAs to mediate RNAi of Gab2.

The invention further encompasses a method for preventing or treating a disorder associated with Gab2 overexpression in a subject in need thereof, comprising administering at least one Erk signaling pathway inhibitor to the subject. When the Erk signaling pathway inhibitor is administered to the subject, the disorder associated with Gab2 overexpression is prevented or treated. In a particular embodiment, the Erk signaling pathway inhibitor prevents Erk activation. Suitable Erk signaling pathway inhibitors include, but are not limited to, inhibitors of Erk activation, for example, inhibitors comprising isolated RNA that mediates RNA interference against mRNA of a component of an Erk signaling pathway (e.g., SHP-2, Ras, Raf, Mek, Erk), inhibitors of Erk and kinases that act upstream of Erk (e.g. Ras inhibitors, Raf inhibitors, Mek inhibitors), several of which are commercially available. In one embodiment, the disorder associated with Gab2 overexpression is selected from the group consisting of leukemia, prostate cancer, ovarian cancer and breast cancer. In a particular embodiment, the disorder associated with Gab2 overexpression is breast cancer (e.g., a breast cancer associated with a Gab2 gene amplification, a breast cancer associated with ERBB2/HER2/Neu overexpression or a breast cancer associated with the expression of a ERBB2/HER2/Neu oncogenic mutant protein).

The invention also relates to a method for preventing or treating a disorder associated with Gab2 overexpression in a subject in need thereof, comprising administering at least one agent that inhibits or eliminates Gab2 expression in one or more cells of the subject. By inhibiting or eliminating the expression of Gab2 in one or more cells of the subject, the disorder associated with Gab2 overexpression is prevented or treated. In one embodiment, the disorder associated with Gab2 overexpression is selected from the group consisting of leukemia, prostate cancer, ovarian cancer and breast cancer. In another embodiment, the disorder associated with Gab2 overexpression is a breast cancer associated with a Gab2 gene amplification.

The invention also provides a method for diagnosing whether a subject has or is at risk for developing a disorder associated with altered Gab2 expression (e.g., Gab2 gene expression, Gab2 protein expression), comprising detecting the expression of a Gab2 gene product (e.g., a nucleic acid transcript, a protein) in a sample from the subject. According to this method, an increase or decrease in the expression of a Gab2 gene product in the sample from the subject, relative to the expression of a corresponding Gab2 gene product in a suitable control sample, is indicative of the subject having or being at risk for developing a disorder associated with altered Gab2 expression. In one embodiment, the Gab2 gene product is a Gab2 protein. In a further embodiment, the Gab2 protein is overexpressed. Suitable techniques for detecting the expression of Gab2 protein in this embodiment include immunohistochemical staining (IHC), Western blot analysis, enzyme-linked immunosorbent assay (ELISA) and radioimmunoassay (RIA), among others. In one embodiment, the disorder associated with altered Gab2 expression is selected from the group consisting of leukemia, prostate cancer, ovarian cancer and breast cancer. In a particular embodiment, the disorder associated with altered Gab2 expression is breast cancer (e.g., a breast cancer associated with overexpression of Gab2 protein, a breast cancer associated with a Gab2 gene amplification, a breast cancer associated with ERBB2/HER2/Neu overexpression or a breast cancer associated with the expression of a ERBB2/HER2/Neu oncogenic mutant protein).

The invention also relates to a method for diagnosing whether a subject has or is at risk for developing a disorder associated with Gab2 overexpression, comprising testing one or more cells from the subject for the presence of a Gab2 gene amplification. The presence of a Gab2 gene amplification in one or more cells from the subject is indicative of the subject having or being at risk for developing a disorder associated with Gab2 overexpression. In one embodiment, the disorder associated with Gab2 overexpression is selected from the group consisting of leukemia, prostate cancer, ovarian cancer and breast cancer. In a particular embodiment, the disorder associated with Gab2 overexpression is breast cancer (e.g., a breast cancer associated with a Gab2 gene amplification, a breast cancer associated with ERBB2/HER2/Neu overexpression or the expression of a ERBB2/HER2/Neu oncogenic mutant protein). The presence of a Gab2 gene amplification can be tested by determining the Gab2 gene copy number in one or more cells from the subject. In one embodiment, a Gab2 gene copy number that is greater than 2 is indicative of the presence of a Gab2 gene amplification. In a related embodiment, a Gab2 gene copy number that is at least two-fold greater in cells from the subject that are affected with the disorder, relative to normal control cells from the same subject, is indicative of the presence of a Gab2 gene amplification. In a certain embodiment, the Gab2 gene copy number is determined in one or more cells of the subject using a nucleic acid probe comprising a Gab2-specific nucleotide sequence. Suitable techniques for determining Gab2 gene copy number in this embodiment include, but are not limited to, in situ hybridization (e.g., fluorescent in situ hybridization).

The invention also encompasses a kit for diagnosing a disorder associated with altered Gab2 expression. The kit can comprise a Gab2-specific antibody and/or a nucleic acid probe that comprises a Gab2-specific nucleotide sequence. In one embodiment, the kit comprises a Gab2-specific antibody. Such antibodies can be used to detect Gab2 protein expression (e.g., by IHC, by Western blotting, by ELISA, by radioimmunoassay (RIA). In another embodiment, the kit comprises a nucleic acid probe that comprises a Gab2-specific nucleotide sequence that further comprises a detectable label (e.g., a fluorophore). Such probes can be used to detect Gab2 mRNA expression and/or Gab2 gene copy number (e.g., by in situ hybridization, by Northern blotting, by Southern blotting). In one embodiment, the disorder associated with altered Gab2 expression is selected from the group consisting of leukemia, prostate cancer, ovarian cancer and breast cancer. In a particular embodiment, the disorder associated with Gab2 overexpression is breast cancer (e.g., a breast cancer associated with Gab2 protein overexpression, a breast cancer associated with a Gab2 gene amplification, a breast cancer associated with ERBB2/HER2/Neu overexpression or a breast cancer associated with the expression of a ERBB2/HER2/Neu oncogenic mutant protein).

### BRIEF DESCRIPTION OF THE DRAWINGS

FIG. 1 is a schematic of the HPLC of Lys-C fragments from Gab2. Nine peaks (numbered) were selected for Edman sequencing.

FIG. 2 depicts the predicted protein sequence derived from Gab2 cDNA (SEQ ID NO: 5). The locations of 8 of the 9 peptides obtained by Edman sequencing are underlined. PH domain is in bold, tyrosine-containing motifs are indicated (−−), and PXXP motifs are in bold.

FIG. 3 is a schematic illustration comparing Dos, Gab2 (p97) and Gab1. Percentage sequence identity among family members is indicated. (P)=potential proline-rich domains.

FIG. 4 depicts the alignment of Gab/Dos family PH domains (top) and the MBD region in Gab1 and Gab2 (p97)(bottom). β1 and β2 sheets corresponding to the PH domain of PLCδ1 are indicated. Basic amino acid residues between β1 and β2 are in bold. A proposed consensus that may specify Gab/Dos family members is indicated. Two PXXP motifs within Gab1 are overlined.

FIG. 5A-FIG. 5E depict the effects of the SHP-2/Gab2 complex on IL-3-induced transactivation, and IL-3-induced MAPK activation. (A) Effects of expression of Gab2ΔY2HA on IL-3-induced c-fos promoter activity. (B) Effects of Gab2/ΔY2HA on IL-3 induced c-fos promoter activity. (C) Effects of point mutations in Gab2 on IL-3-induced c-fos promoter activity. (D) Normalized GAL-4 luciferase activity of BaF3 cells transiently co-transfected with the indicated expression vectors or vector alone, together with a GAL4-Elk-1 construct and GAL-4- and TK-Renilla luciferase reporters. (E) Effects of Gab2/SHP-2 on STAT-mediated activation.

FIG. 6 depicts the nucleotide sequence of Gab2 (SEQ ID NO: 6).

FIG. 7 depicts the targeting strategy for the generation of Gab2 knockout mice.

FIG. 8 depicts surface expression of Fcε RI in Gab2−/− BMMCs.

FIG. 9 depicts Fcε RI-mediated degranulation in Gab2−/− BMMCs.

FIG. 10 depicts Fcε RI-evoked TNFα and IL-6 gene expression in Gab2−/− BMMCs.

FIG. 11 depicts passive cutaneous anaphylaxis in wild-type (WT) and Gab2−/− mice.

FIG. 12 depicts a schematic illustration of the Gab2 response to Fcε RI receptor stimulation.

FIG. 13A depicts the effects of Gab2 overexpression on acinar structure in MCF10A cells. Representative confocal images of cross-sections through the middle of day 20 acini are shown for control (pBabe) and Gab2-overexpressing cells (Gab2). Cultures were immunostained for E-cadherin (top panels) and laminin-5 (bottom panels). Nuclei were counterstained with TO-PRO3.

FIG. 13B depicts enhanced proliferation of MCF10A cells cultured in three dimensions caused by Gab2 overexpression. Representative confocal images of cross-sections of acini structures are shown for control (pBabe) and Gab2-overexpressing cells (Gab2). Acini were immunostained for Ki67, and nuclei were counterstained with TO-PRO3. The graph shows fold changes in cell number. Asterisk (*) indicates P<0.0001.

FIG. 13C depicts increased apoptosis of MCF10A cells caused by Gab2 overexpression. The left panels show representative confocal images of cross-sections of acini that were immunostained for the activated caspase-3 and counterstained with TO-PRO3. The graph shows fold changes in cell number. Asterisk (*) indicates P=0.003.

FIG. 13D depicts the effects of Gab2 overexpression on MCF10A cell number. Cells numbers were counted on the indicated days for control (pBabe) and Gab2-overexpressing cells (Gab2).

FIG. 13E depicts lumenal filling caused by coexpressing Gab2 with antiapoptotic oncogenes. Images show equatorial cross-sections of day 15 MCF10A acini that express the genes indicated above each panel. Acini were immunostained with antibody to laminin-5 and nuclei were counterstained with TO-PRO3. D1=cyclin D1.

FIG. 14A depicts a schematic illustration of NeuNT showing various protein domains (bottom), as well as tyrosyl phosphorylation sites (top; YA, YB, YC, YD, YE) that are present in activated NeuNT and/or various NeuNT mutants.

FIG. 14B depicts immunoblots of Neu or Gab2 immunoprecipitates (IP:Neu, IP:GAB2) that were subjected to immunoblotting (IB) with the indicated antibodies (tyrosine phosphorylated Gab2 (pTyr), GAB2, Neu). The expression of NeuNT and NeuNT mutants (NYPD, YA, YB, YC, YD, YE) in lysates was detected by Neu antibodies.

FIG. 14C depicts immunoblots of Neu immunoprecipitates (IP:Neu) from cells expressing Gab2 (+Gab2) or empty vector (+vector) that were immunoblotted (IB) with the antibodies indicated at the left of each panel (Neu, GAB2). The expression of NeuNT and NeuNT mutants (NYPD, YA, YB, YC, YD, YE) in lysates were detected by Neu antibodies.

FIG. 14D depicts NeuNT enhancement of Gab2 tyrosyl phosphorylation. The figure shows immunoblots of Gab2 immunoprecipitates (IP:GAB2) from parental MCF10A cells (pBabe) or MCF10A cells overexpressing Gab2 and/or Neu (left panel) or lysates from transgenic mouse tumors expressing NeuNT or Gab2+NeuNT (right panel). The immunoblots were probed with the indicated antibodies (tyrosine phosphorylated Gab2 phosphotyrosine antibodies (pTyr) or Gab2-specific antibodies to control for loading (GAB2)). Asterisk (*) indicates nonspecific bands. The arrowhead indicates the position of tyrosyl-phosphorylated Gab2.

FIG. 14E depicts cooperation of Gab2 with Neu to induce invasive multiacinar structures. Phase and confocal images of day 15 acini are shown for cells expressing pBabe, Gab2, Neu, or a combination of Gab2 and Neu, as indicated. Images after 48 d of culture (right) emphasize the invasive nature of acini overexpressing both Gab2 and Neu (bottom right panel). Acini were immunostained with antibody to laminin-5 and nuclei were counterstained with TO-PRO3. Arrows indicate multiacinar structures with disrupted architecture.

FIG. 15A depicts the role of SHP-2 binding in Gab2-evoked proliferation of MCF10A cells. The immunoblot (left) shows expression Gab2 and Erk2 (loading control) in MCF10A cells expressing either control vector (pBabe), wild-type Gab2 (GAB2), the mutant Gab2 protein 3YF or the mutant Gab2 protein DM, which does not bind SHP-2. The middle panels show images of MCF10A cells that were cultured in three dimensions for 15 d, and stained for Ki67. Nuclei were counterstained with TO-PRO3. The graph (right) indicates proliferation (fold increase in cell number) in each of the indicated cell lines, as assessed by Ki67 staining. Results represent mean±s.e.m. from three independent experiments. Asterisk (*) indicates P<0.01 by ANOVA.

FIG. 15B depicts antagonism of Gab2-evoked proliferation due to the effects of PTPN11-mediated Shp2 knockdown. The immunoblot (left) shows the expression of Gab2, Shp2 and Erk2 (loading control) in MCF10A cells expressing either control vector (pBabe) or wild-type Gab2 (GAB2). These cells were infected with either PTPN11 shRNA (PTPN11 RNAi) or pSuper control virus. PTPN11 shRNA reduced Shp2 expression by ˜70%. The graph (right) show fold increase in cell number to assess proliferation. Asterisk indicates *P<0.001 by ANOVA.

FIG. 15C depicts a graph illustrating that Shp2 binding sites are required for Gab2-Neu cooperation. MCF10A cells expressing the control vector (pBabe), wild-type Gab2 (GAB2), mutant Gab2 DM (DM), mutant Gab2 3YF (3YF), Neu, or a combination of Neu and wild-type (Neu+GAB2) or mutant Gab2 (Neu+DM, Neu+3YF), were cultured in three dimensions and multiacinar structures quantified on day 7. Results represent means±s.e.m. from two independent experiments. Asterisk (*) indicates P<0.0001 by ANOVA.

FIG. 15D depicts Erk activation by immunoblotting (top), showing that Shp2 binding sites in Gab2 are required for enhanced Erk activation as a consequence of Gab2 overexpression. Lysates were obtained from MCF10A cells infected with and expressing the indicated vectors (control vector (pBabe; Neu (Neu); wild-type Gab2 (GAB2)). Cells were cultured in three dimensions, starved and stimulated for the indicated times (0′, 5′, 15′, 60′). Immunoblots were probed with phospho-Erk2 (PERK) and Erk-2 specific (ERK2) antibodies. The graph with numbers (bottom) indicates the ratio of phosphorylated Erk2/Erk2 intensity (arbitrary units) for the sample in each corresponding lane in the immunoblot above and represent one of three independent experiments with similar results.

FIG. 15E depicts Erk activation by immunoblotting (top), showing that Shp2 binding sites in Gab2 are required for Erk activation. Lysates were obtained from MCF10A cells infected with and expressing the indicated vectors (control vector (pBabe; Neu (Neu); wild-type Gab2 (GAB2); mutant Gab2 3YF (3YF); mutant Gab2 DM (DM)). Cells were cultured in three dimensions, starved and stimulated for the indicated times (0′,15′). Immunoblots were probed with phospho-Erk2 (pERK) and Erk-2 specific (ERK2) antibodies. The graph with numbers (bottom) indicates the ratio of phosphorylated Erk2/Erk2 intensity (arbitrary units) for the sample in each corresponding lane in the immunoblot above and represent one of three independent experiments with similar results.

FIG. 15F depicts a graph showing that Mek inhibition blocks Gab2-evoked proliferation and cooperation of Gab2 and Neu. MCF10A cells expressing control vector (pBabe) or Gab2 (GAB2) were either treated with UO126 (UO 15 μM) on days 13 and 14 or were left untreated (Untreated). Proliferation was assessed on day 15 by positive Ki67 staining. Results represent means±s.e.m. from two independent experiments. Asterisk (*) indicates P<0.005, two asterisks (**) indicate P<0.0003.

FIG. 15G depicts representative phase images of acini at 9 days in untreated MCF10A cells (left panels) and MCF10A cells that were treated with the Mek inhibitor, UO126, on days 1 and 2.

FIG. 16 depicts immunoblots showing Gab2 expression in retrovirally-transduced MCF10A cells. Lysates of MCF10A cells expressing HER2 alone (HER2), or HER2 in combination with wild-type Gab2 (HER2+Gab2) or mutant Gab2 proteins (HER2+3YF or HER2+DM), were immunoblotted with HER2, Gab2 and Erk2 antibodies, as indicated at the left of each panel.

FIGS. 17A-H depict Gab1-mediated promotion of multi-acinar structure formation via Shp2. Confocal images showing the formation of multi-acinar structures at day 15 in MCF10A cells are shown. Acini were immunostained for E-cadherin and the nuclei were counterstained with TO-PRO3. (A) MCF10A cells expressing pBabe; (B) MCF10A cells expressing wild-type Gab2 (Gab2); (C) MCF10A cells expressing Gab2 mutant 3YF (3YF); (D) MCF10A cells expressing Gab2 mutant DM (DM); (E) MCF10A cells expressing HER2 (HER2); (F) MCF10A cells expressing HER2 and wild-type Gab2 (HER2+Gab2). Arrows indicate acini having disrupted basement membrane and an invasive structure.; (G) MCF10A cells expressing HER2 and Gab2 mutant 3YF (HER2+3YF); (H) MCF10A cells expressing Gab2 mutant DM (HER2+DM). Arrows indicate acini having disrupted basement membrane and an invasive structure.

FIG. 18 depicts immunoblots showing that Gab2 overexpression does not affect Stat5 activation in MCF10A cells in 3D cultures. MCF10A cells were infected with pBabe, Gab2, HER2, or a combination of Gab2 and HER2, as indicated above each lane. The cells were stimulated with assay media for the indicated times (0′, 15′, 60′). Lysates were immunoblotted with antibodies against phospho-Stat5 (pStat5) and reprobed with antibodies against Stat5.

FIG. 19 depicts immunoblots showing that Gab2 expression has minimal effects on Akt activation in MCF10A cells in 3D cultures. MCF10A cells were infected with pBabe, Gab2, HER2, or a combination of Gab2 and HER2, as indicated above each lane. The cells were stimulated with assay media for the indicated times (0′, 15′, 60′). Lysates were immunoblotted with antibodies against phospho-Akt (pAkt) and reprobed with antibodies against Akt.

FIG. 20 depicts immunoblots showing the effects of different expression levels of wild type and mutant Gab2 in MCF10A cells. Lysates of MCF10A cells expressing pBabe, HER2 alone (HER2), or HER2 in combination with wild-type Gab2 (HER2+Gab2) or mutant Gab2 proteins 3YF or DM (HER2+3YF or HER2+DM, respectively), were immunoblotted with Her2, Gab2 and Erk2 antibodies, as indicated at the left of each panel.

FIG. 21A depicts a schematic illustration (top) of the MMTV-Gab2 transgenic construct showing relevant features and immunoblots (bottom) using the antibodies indicated at the left of the images on cell lysates from mammary glands of nontransgenic mice (FVB), or mice from the indicated MMTV-NeuNT Gab2 transgenic lines (line 1, line 2, line 3), as indicated above each lane.

FIG. 21B depicts Kaplan-Meier curves showing onset of mammary tumors in Gab2-overexpressing mice (Gab2), NeuNT-expressing mice (NeuNT; n=42) and NeuNT Gab2 mice (line 1, n=45; line 2, n=16; and line 3, n=25). P=0.011 for NeuNT versus NeuNT Gab2 1; P<0.0001 for NeuNT versus either NeuNT Gab2 line 2 or line 3 by log-rank test.

FIG. 21C depicts whole-mount (top panels) and H&E-stained sections (bottom panels) of mammary glands from 3-month-old virgin MMTV-NeuNT (NeuNT) or MMTV-NeuNT Gab2 (line 3; NeuNT+Gab2) mice (n=3) showing the effects of overexpressing Gab2 on NeuNT-evoked tumorigenesis in mice. Whole mounts and sections are stained with hematoxylin and eosin. An enlarged view of the boxed area in the top middle panel is shown in the top left panel.

FIG. 21D depicts Kaplan-Meier curves showing tumor onset in MMTV-NeuNT mice with the indicated Gab2 genotypes (right of graph), indicating that endogenous Gab2 is required for NeuNT-evoked mammary tumorigenesis.

FIG. 21E depicts the decrease of Erk activation and cell proliferation of a NeuNT-transformed cell line caused by Gab2 knockdown. (Left panels) Cell lysates from vector control (Control) and Gab2 knockdown cells (Gab2 RNAi) were immunoblotted with the indicated antibodies (Gab2, phopho-Erk, Erk2). Graphic results (right) represent cell number for the pooled values from three independent experiments (means±s.e.m.). *P=0.004, **P=0.007, ***P=0.008. Equal numbers of vector control and Gab2 shRNA-expressing cells were plated and cell numbers were counted on the indicated days.

FIG. 22 depicts whole mount mammary glands from virgin 19 week-old (mature puberty) FVB non-transgenic (left panel) and MMTV-Gab2 mice from transgenic lines 1 (middle panel) and 3 (right panel) that have been stained with hematoxylin and eosin (n=3).

FIG. 23A depicts immunoblots showing the phosphorylation status of Erk in normal pre-neoplastic mammary gland samples from both MMTV-NeuNT: Gab2^{+/+} and MMTV-NeuNT: Gab2^{-/-} mice. Immunoblots were probed with antibodies against the proteins indicated at the right of each panel (phospho-Erk (pErk), Erk2, Keratin). Note that loss of Gab2 results in a dramatic decrease in Erk activation in the preneoplastic mammary glands.

FIG. 23B depicts immunoblots showing the phosphorylation status of Erk in breast tumor samples from both MMTV-NeuNT Gab2^{+/+} and MMTV-NeuNT: Gab2^{-/-} mice. Immunoblots were probed with antibodies against the proteins indicated at the right of each panel (phospho-Erk (p-Erk), Erk2, Keratin).

FIG. 24A depicts Gab2 amplification and overexpression in human breast cancer. Representative images of FISH analyses using a Gab2 DNA probe on breast tumor cells from two primary breast tumors that overexpress Gab2 (Tumor, lower panels) and adjacent normal stromal cells (Normal stroma; upper left panel) are shown. The upper right panel shows an image of Gab2 FISH on a metaphase spread of normal chromosomes from human lymphocytes. Gab2 hybridization spots are orange. Nuclei and chromosomes are counterstained with DAPI.

FIG. 24B depicts immunohistochemical staining of Gab2 protein in sections of mammary tissue from breast tumors. The upper panels show cells from tumors having Gab2 gene amplification. The upper left and right panels correspond to Sample 2 and Sample 3 in Table 1, respectively. The lower panels show Gab2 protein expression in two tumors identified as having low Gab2 expression by microarray analyses.

### DETAILED DESCRIPTION OF THE INVENTION

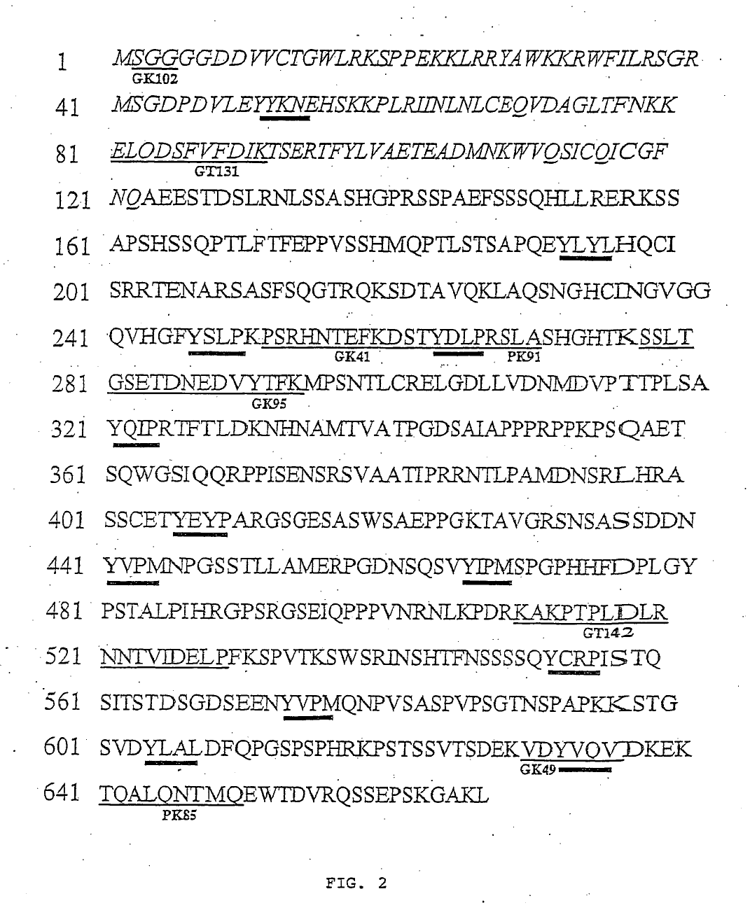

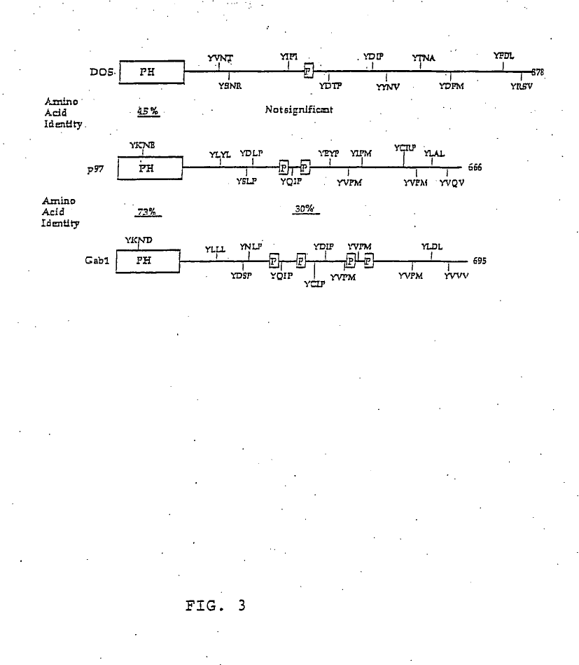

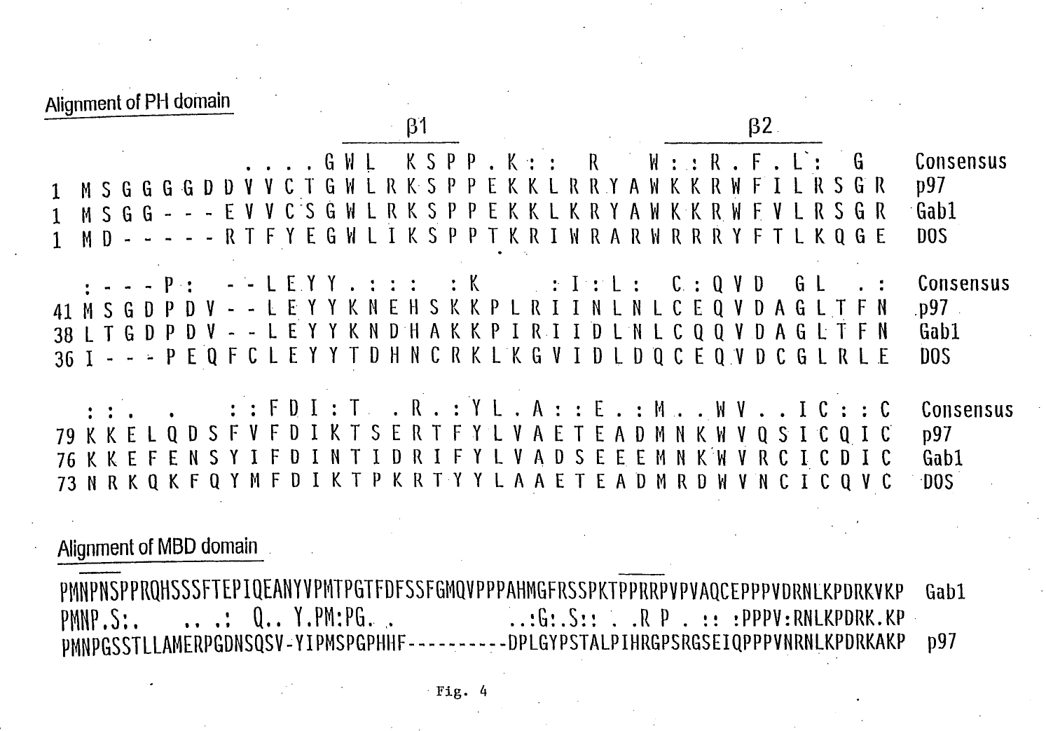

Gab2, a novel member of the DOS/Gab1 subfamily of scaffolding molecules, was purified, cloned and characterized. DOS/Gab1 Family members contain an N-terminal PH (Pleckstrin homology) domain, proline-rich motifs and multiple tyrosines, but show minimal sequence identity, mainly in their PH domains (FIGS. 2, 4) (see Rameh, L. E. et al., J. Biol. Chem. 272, 22059-22066 (1997)). Most of their tyrosyl residues, including those required for SHP-2 (also referred to herein as Shp2 and Shp-2) binding (Table 2 and FIG. 3), occur at similar relative positions and within similar sequence contexts (FIG. 3). This suggests that Gab/Dos proteins bind similar signal relay molecules, which may have to bind in a specific orientation. IRS proteins and FRS-2 have similar topography, but are distinguished from Dos/Gab proteins by their PH domains and the order and sequence contexts of their tyrosines and proline-rich motifs. Members of the family include Drosophila Dos and mammalian Gab1. Tissue-specific differences in Gab1 and Gab2 expression exist, and at least one functionally important region of Gab1, the MBD, is divergent in Gab2. Although Gab2 and Gab1 are only distantly related and have distinct functions in vivo, the novel protein is termed Gab2 to simplify the nomenclature. Gab2 is also known as GAB2, p97 and p97/Gab2.

Extracellular stimuli, such as cytokines, growth factors, hormones and antigens, regulate cell proliferation and differentiation via changing the tyrosine phosphorylation states of proteins, such as Gab2, inside the cell. Extracellular receptors, such as cytokine receptors, trigger multiple signaling cascades, which regulate cell proliferation and differentiation. Binding of a cytokine to its cognate receptor activates receptor-associated Janus family (e.g., Jak/Tyk) protein-tyrosine kinase(s) (PTKs), resulting in their phosphorylation and the phosphorylation of receptor cytoplasmic domains. Phosphorylation creates docking sites for SH2 domain-containing signal relay molecules, most of which are substrates for Janus PTKs. Signal relay molecules promote activation of downstream cascades, including the Ras/Raf/mitogen-activated protein Kinase (MAPK), phosphatidylinositol-3′ kinase (PI-3K) and Stat cascades. Ultimately, these cascades evoke the transcription of immediate-early genes, such as c-fos (see Ihle, J. N. et al., Stem Cells 15, 105-111 (1997)). Receptor tyrosine kinases (RTKs) and multi-chain immune recognition receptors (MIRRs) utilize analogous signaling strategies.

Gab2 is widely expressed in a variety of tissues, and contains multiple potential serine/threonine phosphorylation sites. Upon cytokine, growth factor, hormone or antigen receptor stimulation, Gab2 becomes tyosine phosphorylated and involved in the activation of multiple signaling cascades via interaction with different intracellular signaling molecules. Upon tyrosine phosphorylation, Gab2 recruits SH2-containing downstream signal molecules. Gab2 may also transmit extracellular stimuli through interaction with cellular proteins containing SH3 or WW domains. In addition, Gab2 may exert its effect through an interaction with intracellular lipids.

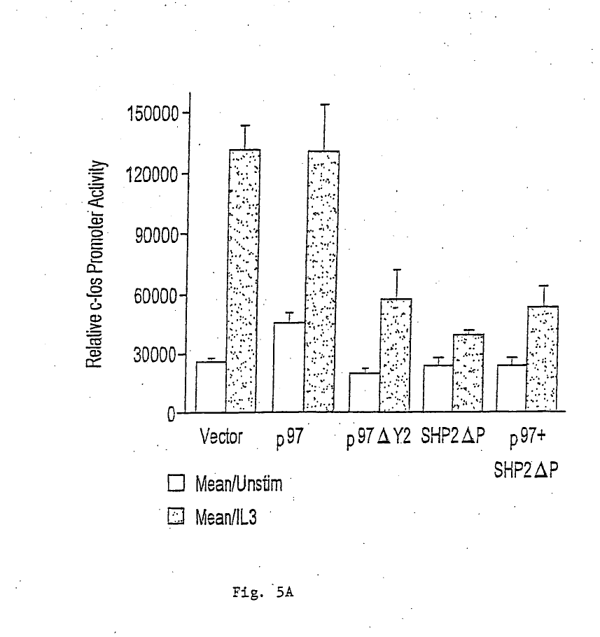

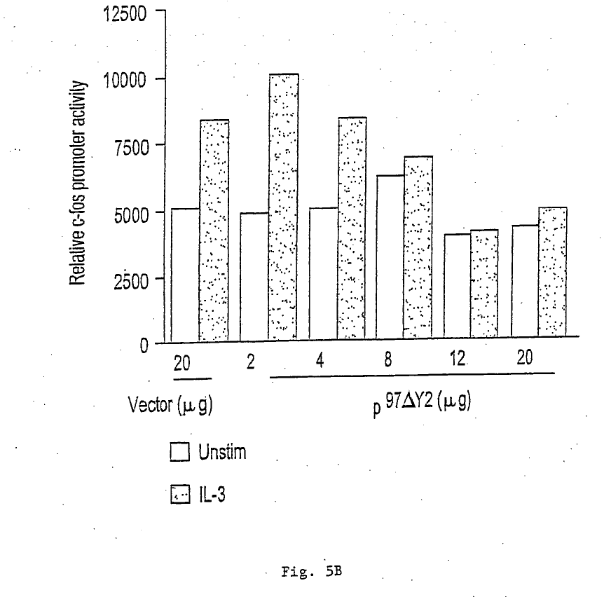

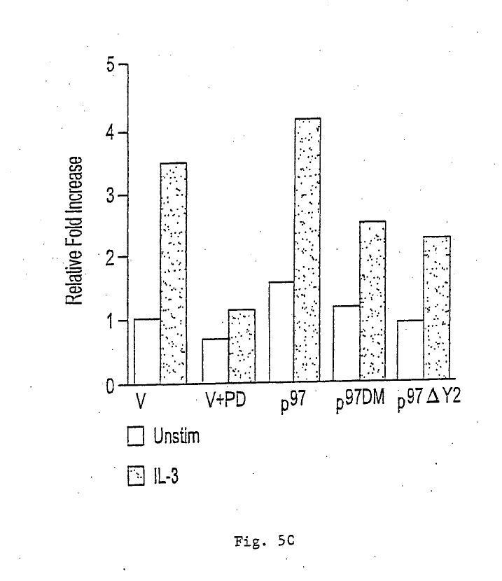

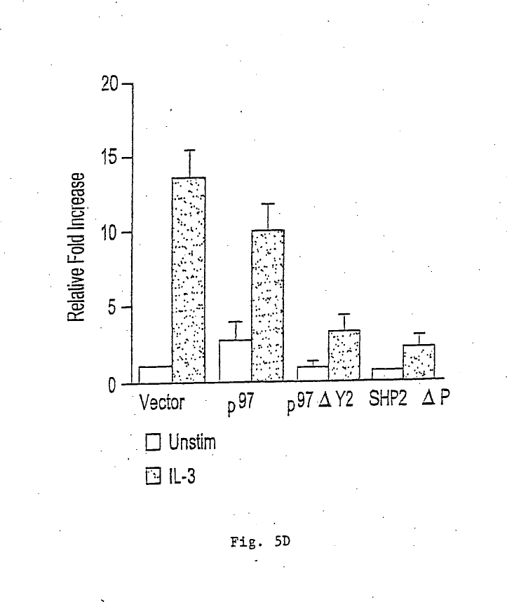

In various hematopoietic cell lines, in response to cytokine stimulation and engagement of MIRRs, Gab2 becomes associated with various SH2-containing molecules, including SHP-2, p85 (the regulatory subunit of PI-3K) and Shc. Gab2, via its interaction with SHP-2, is required for cytokine (e.g., IL-3) induced c-fos gene transcription via a novel mechanism that is parallel to or downstream of MAPK FIGS. 5A-E). However, Gab2 can also signal to the Erk pathway and, under some conditions, the MAPK cascade under some conditions. For the MAPK cascade, this signal does not require SHP-2 binding since Gab2 mutants which do not bind SHP-2 potentiate (do not inhibit) MAPK activation. Conceivably, Gab2 may transmit signals to the MAPK cascade via PI-3K.

In several systems, PI-3K functions downstream of Ras and upstream of MAPK particularly under conditions of limiting receptor stimulation. The Gab2/p85 complex is critical for PI-3K activation evoked by cytokine receptors, such as IL-3/GM-CSF and IL-2, which do not contain p85-binding sites. However, Gab2 is also tyrosyl phosphorylated upon stimulation of receptors that bind p85 directly (e.g., CSF-1R) or have other means of p85 recruitment (e.g., CD19 for the BCR). In these systems, Gab2 may amplify PI-3K activation.

Upon cytokine stimulation, Gab2 can be recruited to receptor complexes. PH domains bind to phosphatidylinositol lipids, providing a potential recruitment mechanism. Alternatively, the Gab2 region corresponding to the Gab1 MBD may direct binding. A third possibility is that recruitment is indirect. Mutation of Y577 in the IL-3 receptor βc chain, the Shc binding site, severely diminishes Gab2 tyrosyl phosphorylation (see Itoh, T. et al., J. Biol. Chem. 271, 7587-7592(1996)). Since Grb2 (presumably via its SH3 domains) binds Gab2 constitutively, and the Grb2 SH2 domain binds to tyrosyl phosphorylated Shc, Gab2 recruitment to the IL-3 receptor may occur via a Shc-Grb2 complex.

Role of Gab2 in Allergic Responses

Interaction of multivalent antigen with IgE-bound mast cells provokes several effects: the immediate release of preformed granules containing vasoactive amines including histamine and serotonin (degranulation), secretion of lipid mediators, and the late synthesis and release of cytokines. Fcε RI is the high affinity receptor for IgE. Fcε RI consists of a ligand binding α chain, one β, and two γ chains. Crosslinking Fcε RI by the multivalent antigen activates the β chain associated with tyrosine kinase, lyn. Activated lyn phosphorylates the tyrosine-based activation motifs (IAMTs) in the cytoplasmic domains of the β and γ chains, which recruit and activate tyrosine kinase Syk. Subsequently, lyn and syk phosphorylate various signal relay molecules, resulting in the activation of multiple downstream signaling cascades including phosphatidylinositol 3-kinase (PI-3K) and three major subfamilies of mitogen-activated protein kinases (MAPKs), Erk, JNK, and p38. The lipid products of PI-3K are required for full activation of Tec family tyrosine kinase Btk/Emt, and subsequent phosphorylation and activation of PLCγ. Activated PLCγ then converts PI4, 5P2 into inosital 1,4,5-triphosphate (IP3) and diacylglycerol (DAG), which can increase intracellular Ca⁺⁺ level and activate PKC respectively. Both Ca⁺⁺ and PKC are required for the optimal degranulation. Activation of Erk, JNK, and p38 are important for the late phase cytokine production.

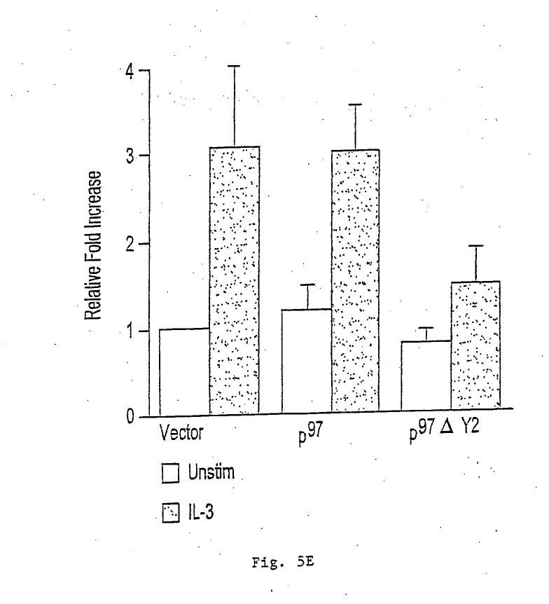

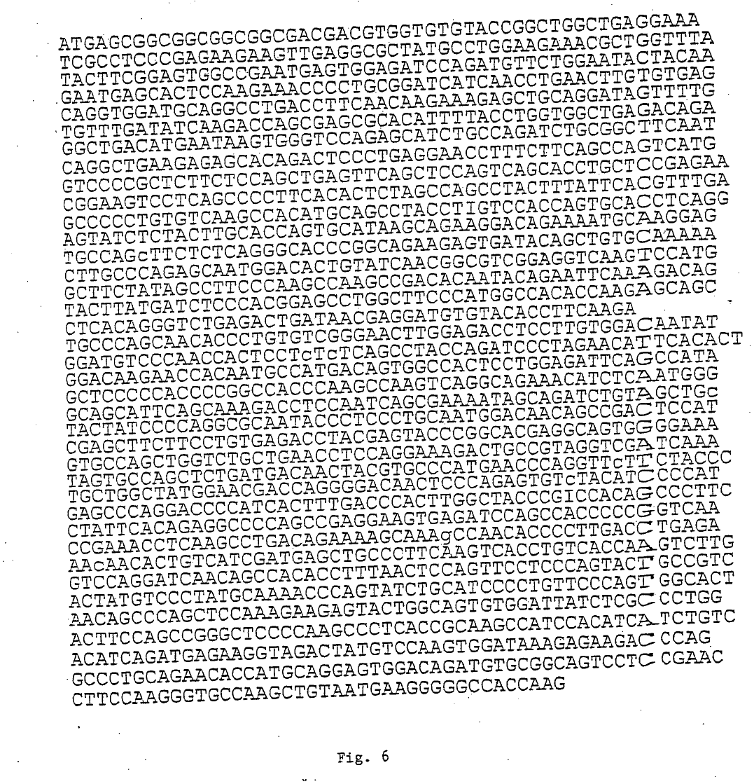

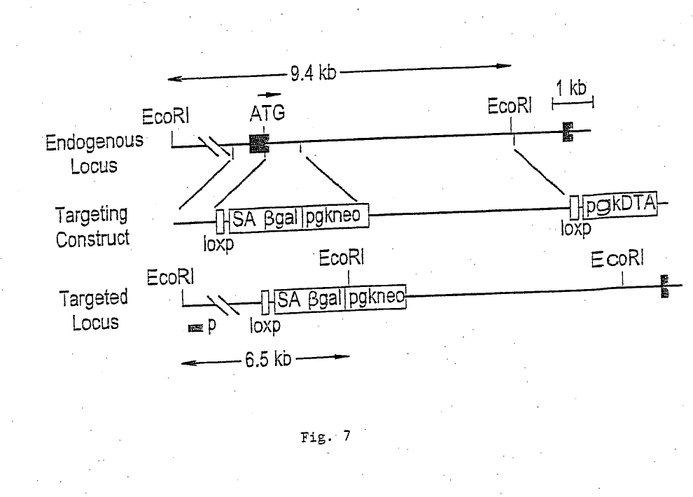

Since one of the major functions of mast cells is involved in IgE-associated immune responses, the role of Gab2 in signaling evoked by Fcε RI in Bone Marrow-derived Mast Cells (BMMCS) was determined. Gab2 knockout mice were generated by conventional gene targeting methods (FIG. 7). Bone marrow mast cells or macrophages were isolated from these mice by standard techniques. To examine Fcε signaling, mast cells were loaded with anti-DNP IgE and then stimulated with a range of concentrations of DNP, and serotonin release was quantified. For assessment of macrophage Fcγ responses, bone marrow macrophages were presented with opsonized RBCs, and phagocytosis was quantified by microscopy. In addition, various biochemical parameters were monitored by standard techniques.

In contrast to Gab1 knockout mice, which are embryonic lethal, Gab2 homozygous (−/−) knockout mice are healthy, fertile and appear to live a normal life span. However, analysis of mast cells from Gab2−/− mice show that they are refractory to stimulation through IgE receptors which are the main receptors mediating allergic responses. Mast cell numbers are also diminished in Gab2−/− mice, and Fc gamma receptor signaling in macrophages is defective. Thus, elimination of Gab2 expression, or Gab2 interaction with key signaling molecules, is a powerful approach to prevention of allergic responses.

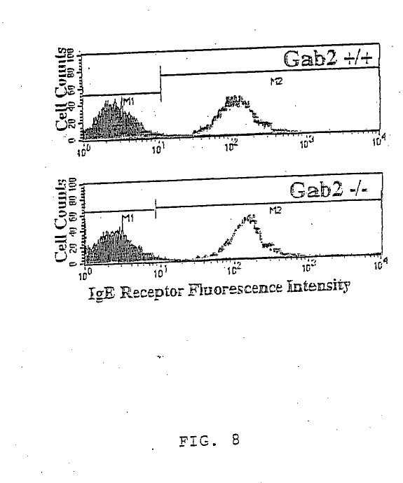

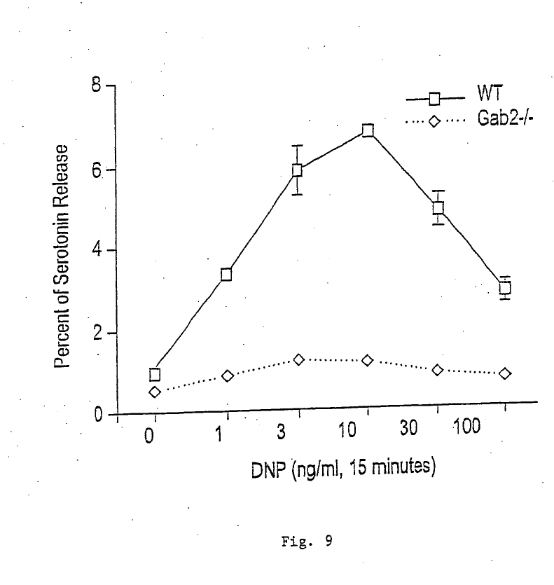

The mast cell degranulation evoked by Fcε RI crosslinking was dramatically impaired (e.g., a 3-7 fold impairment of Fcε-evoked serotonin release) in Gab2−/− BMMCS compared to wild type BMMCS (FIG. 9). Fcε RI-evoked PLCγ phosphorylation and interleukin-3-induced AKT activation also are significantly diminished in Gab2−/− BMMCS, as well as Fcε RI evoked TNFα and IL-6 gene expression (FIG. 10). Furthermore, activation of JNK and p38 are defective in Gab2−/− upon Fcε RI crosslinking. In addition, Gab2−/− mice show decreased passive cutaneous anaphylaxis compared to wild mice (FIG. 11). These data show that Gab2 is required for selective events downstream of Fcε activation, most likely those dependent on PI-3K activation.

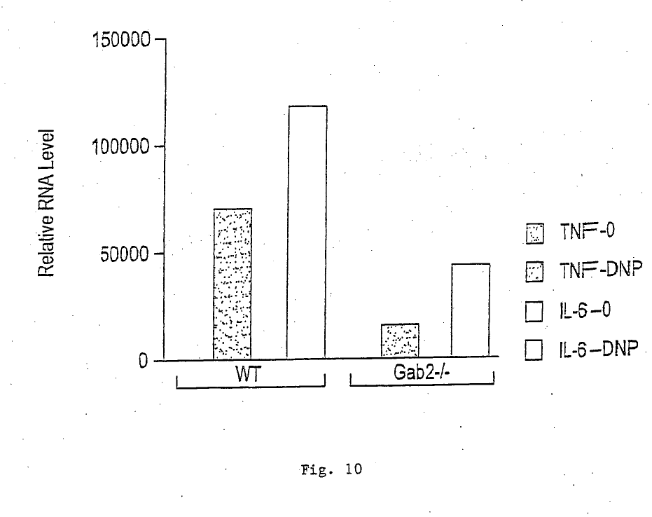

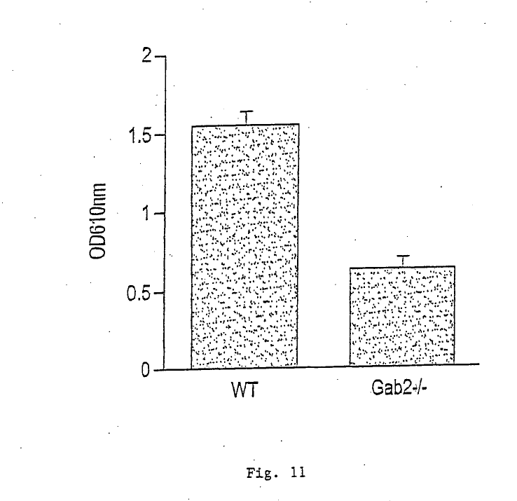

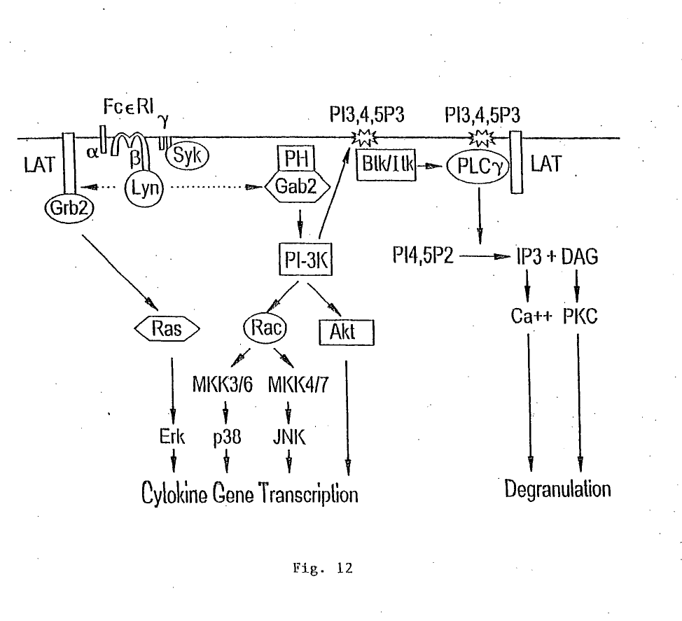

These defects in Fcε RI-evoked biological response and signaling cascades in Gab2−/− BMMCS are not due to a change in surface expression of Fcε RI (FIG. 8) or defective activation of upstream tyrosine kinases. Notably, early events in Fcε responses, including surface expression of the IgE receptor, Syk phosphorylation and, most likely, LAT phosphorylation, as well as Erk MAPK activation, are normal. Furthermore, total tyrosyl phosphorylation in Gab2−/− BMMCs is normal upon FcεRI engagement. Moreover, expression of the wild type Gab2 in Gab2−/− BMMCS can rescue the FcεRI-evoked signaling defects. Thus, these data demonstrate that Gab2 plays an important role in FcεRI-evoked signaling and effector function in mast cells and is essential for the IgE-initiated effector function of mast cells by activating the PI-3K/Akt and JNK and p38 cascades (FIG. 12). Similar results are obtained in macrophages, where Fcγ-evoked phagocytosis is impaired by up to 50%. Gab2 and its associated signaling molecules represent new targets for developing drugs to treat allergy.

Role of Gab2 Interaction with SHP-2

In another embodiment, the Gab2/SHP-2 complex also has a distinct and novel signaling role, since Gab2 mutants inhibit activation of c-fos luciferase (FIG. 5A) and Elk (i.e, TCF)-(FIG. 5D) and STAT-(FIG. 5E) driven reporters. The N-SH2 of SHP-2 binds to tyrosyl phosphorylated Gab2, and N-SH2 engagement activates the enzyme (see Barford, D. and Neel, B. G., Structure 6, 249-254 (1998)). Dominant negative SHP-2 blocks Gab2 potentiation of basal c-fos promoter activity (FIG. 5A), suggesting that upon binding to Gab2, activated SHP-2 dephosphorylates one or more targets to permit c-fos activation. The identity of this target(s) remains unknown. Previous experiments with “substrate trapping” mutants suggested that Gab2 is a SHP-2 substrate, and Dos reportedly is a substrate of Csw. However, mutating its SHP-2 binding sites does not increase Gab2 tyrosyl phosphorylation. Conceivably, the increased association of the cystein to serine mutant (C>S) of SHP-2 with tyrosyl phosphorylated Gab2 is due to SHP-2 regulation of a PTK that phosphorylates Gab2, with consequently increased SHP-2 binding via its SH2 domains to Gab2. However, it remains possible that one or more sites on Gab2 are SHP-2 targets. SHP-2 could dephosphorylate its own binding sites. Alternatively, it could target other sites, but the net increase in their phosphorylation might be less than the decreased phosphorylation due to loss of the SHP-2 sites. If sites on Gab2 are not the primary target(s) of the Gab2/SHP-2 complex, then presumably Gab2 directs SHP-2 to the proper location for accessing its target(s).

Although Gab2 is strongly tyrosyl phosphorylated in response to many stimuli, its association with SHP-2 varies, suggesting that Gab2 may be phosphorylated on distinct sites in response to different stimuli. The widespread expression of Gab2 suggests that its involvement in growth factor and/or cytokine signaling cascades in non-hematopoietic cells should be investigated.

SHP-2 is a critical component of multiple signaling cascades. Embryonic stem cells expressing mutant SHP-2 exhibit defective ex vivo hematopoietic differentiation. SHP-2 is required for cytokine-induced MAPK activation. For example, SHP-2 is required for interleukin-5 (IL-5)-induced MAPK activation in eosinophils, interleukin-2 (IL-2)-induced MAPK activation in T cells, and for immediate-early gene activation in response to multiple cytokines in various cellular contexts. RTK and T cell receptor (TCR)-evoked MAPK activation also require SHP-2, and SHP-2 function is necessary for RTK-induced c-fos expression acting, at least in part, to control the activity of the transcription factor Elk-1. Likewise, the Drosophila homolog of SHP-2, Csw, is required for RTK-induced gene induction. These studies suggest that SHP-2 is a required positive component of cytokine, RTK, and MIRR signaling cascades, acting upstream of MAPK, which, in turn, lies upstream of immediate-early genes.

However, several lines of evidence raise questions about this simple linear model. For example, SHP-2 has been reported to act both upstream and downstream of Ras. Genetic analysis indicates that Csw acts upstream and downstream of Ras or functions in a parallel cascade in Sevenless signaling. Biochemical and genetic studies suggest that Csw binds to, dephosphorylates, and signals through the Daughter of Sevenless (Dos) gene product, a scaffolding protein remotely related to mammalian Gab1. SHP-2 binds directly to some growth factor receptors, but in other cascades, it binds to scaffolding molecules such as IRS family members, Gab1, and FRS-2, and/or to one or more transmembrane glycoproteins, such as SHPS-1/SIRPs.

Accordingly, upon cytokine, growth factor, hormone or antigen receptor (B cell receptor/T cell receptor) stimulation, Gab2 becomes tyrosyl phosphorylated and associates with several SH2 domain-containing proteins, including SHP-2. Gab2 via interaction with SHP-2 is required for cytokine induced gene expression, via a novel signaling cascade. Expressed Gab2 mutants that were unable to bind SHP-2 blocks cytokine-induced c-fos promoter activation, inhibiting Elk-1-mediated and STAT5-mediated transactivation, indicating that Gab2 function is required for cytokine-induced immediate early gene activation. In addition, dominant negative SHP-2 inhibits IL-3-induced MAPK activation in BaF3 cells. However, SHP-2 need not bind to Gab2 to participate in MAPK activation, whereas it must bind to Gab2 to allow transcriptional activation (FIGS. 5A-E). Thus, SHP-2 has at least two sites of action in cytokine signaling. The requirement of SHP-2 for MAPK activation could be mediated through another binding protein, e.g, p135 in BaF3 cells (see Gu, H. et al., J. Biol. Chem. 272, 16421-16430 (1997)), or through the reported direct interaction of SHP-2 and Jak-2 (see Fuhrer, D. K. et al., J. Biol. Chem. 270, 24826-24830 (1995); Ali, S. et al., EMBO J 15, 135-142 (1996)), whereas the work herein shows that the requirement of SHP-2 for transactivation is mediated by Gab2. SHP-2 action at multiple steps may help explain some of the discrepancies between earlier studies over the site of action of SHP-2 in RTK signaling. Future experiments can be directed to elucidating Gab-2-dependent and -independent functions of SHP-2 in signaling by cytokines, growth factors and MIRRs. How these interactions culminate in MAPK activation and/or induction of gene expression is unclear. To understand how SHP-2 functions requires both the identification and characterization of its binding proteins and substrates.

Gab2 is an important new regulator of receptor signaling that controls a novel cascade to immediate-early gene activation and suggest new functions for SHP-2 in cytokine receptor signaling. Moreover, since SHP-2 itself is require for IL-3-induced MAPK activation, these data show that SHP-2 is required for at least two steps in cytokine signal transduction, one upstream of, and the other downstream of or parallel to MAPK.

Gab2/SHP-2 may be required for MAPK translocation because MAPKs probably must translocate to the nucleus to phosphorylate transcription factors (e.g., Elk-1) (see Brunet, A. and Pouyssegur, J., Essays Biochem 32, 1-16 (1997)). Consistent with this model, MAPK activation also is required for IL-3-driven STAT reporter activity in BaF3 cells (see Rajotte, D. et al., Blood 88, 2906-2916 (1996)).

Gab2/SHP2 may control a cascade that inhibits dephosphorylation of SRF, TCF, and/or other components of the c-fos transcriptional machinery, perhaps by controlling the serine-threonine kinase KSR. Recent work suggests that KSR activates a serine-threonine phosphatase that catalyzes Elk-1 dephosphorylation. Interestingly, IRS-1 also signals to c-fos without affecting MAPK. Data suggest that this signal maybe sent via IRS-1/SHP-2 complexes; by inference, similar signaling cascades may exist for other scaffolding protein/SHP-2 complexes. Whereas Gab2/SHP-2 complex formation appears to be required for full activity of the c-fos promoter, Gab2 mutants unable to bind SHP-2 only partially inhibit c-fos activation. It is unclear whether partial inhibition is due to incomplete interference with endogenous Gab2 by the Gab2 mutants or instead indicates that Gab2/SHP-2 is predominantly an “amplifier” of c-fos activation. The latter is likely because the effects of the Gab2 mutants on c-fos promoter activity are enhanced at lower levels of IL-3. Moreover, the results described herein do not exclude important roles for Gab2 and/or SHP-2 in cascades other than those leading to c-fos promoter activation.

Role of Gab2 in Neoplastic Disorders

Oncogenes can subvert normal function of Gab2. For example, Gab2 is required for Bcr-Abl-evoked activation of Akt and Erk and, consequently, for transformation by Bcr-Abl (Sattler, M. et al., Cancer Cell 1, 479-492 (2002)), whereas the association of Gab2 with P13K and Shp2 is essential for fibroblast transformation by v-Sea (Ischenko, I. et al., Oncogene 22, 6311-6318 (2003)). Mutations in two Gab2-binding proteins also have key roles in human cancer. Activating mutations in the gene encoding P13K occur in many carcinomas, including breast neoplasms (Samuels, Y. et al., Science 304, 554 (2004)). PTPN11 (the gene encoding SHP2), is normally required for activation of the Ras-Erk cascade by multiple RTKs and cytokine receptors (Feng, G. S., Exp. Cell Res. 253, 47-54 (1999); Neel, B. G. et al., Trends Biochem. Sci. 28, 284-293 (2003)). Gain-of-function mutations in PTPN11 are found in several types of leukemia and some solid tumors (Tartaglia, M. et al., Nat. Genet 34, 148-150 (2003); Bentires-Alj, M. et al., Cancer Res. 64, 8816-8820 (2004)). Recent work suggests that Shp2 mutant proteins cause leukemia by binding Gab2 and enhancing Gab2-dependent signaling (Mohi, M. G. et al., Cancer Cell 7, 179-191 (2005)).

Gab2 may promote breast carcinogenesis, for example, by enhancing EGFR/ErbB2 and/or ErbB3 initiated growth and survival signals. ErbB2 is also referred to herein as ERBB2. ErbB2/Neu, which encodes the RTK HER2 in humans, is the best known gene that is amplified and overexpressed in human breast cancer. Although oncogenic mutant forms of ErbB2 are rarely found in humans, ErbB2 is amplified and overexpressed in 20-30% of breast cancers. Amplification of ErbB2 correlates with aggressive disease and poor prognosis. Transgenic overexpression of Neu (the equivalent of ERBB2 in rats) in mice also causes mammary carcinomas with high penetrance (Muller, W. J. et al., Cell 54, 105-115 (1988); Bouchard, L. et al., Cell 57, 931-936 (1989)). ErbB2/Neu overexpression is also found in ˜90% of Ductal Carcinoma in situ (DCIS), a malignant ductal carcinoma with an intact basement membrane barrier. Under in vitro culture conditions, EGFR (ErbB1) signals have been shown to contribute to disorganized growth of colonies of breast tumor cells under a three-dimensional basement membrane, which mimics DCIS. Furthermore, overexpression of ErbB2 under the control of mouse mammary tumor virus (MMTV) long terminal repeat (LTR) in mammary gland causes mammary tumor formation with fairly long latency in mice. Consistent with a role of ErbB2 in breast cancer, ErbB2 blocking antibodies have been used clinically to reduce regression of some of the ErbB2-overexpressing breast tumors. For example, the therapeutic efficacy of the HER2-specific monoclonal antibody trastuzumab (Herceptin) (Slamon, D. J. et al., N. Engl. J. Med. 344, 783-792 (2001)) provides strong evidence for the functional significance of HER2 overexpression. Collectively, these data indicate that ErbB2/neu overexpression contributes to breast carcinogenesis in human. Notably, Gab2 is tyrosyl phosphorylated upon activation of HER2 (Daly, R. J. et al., Oncogene 21, 5175-5181 (2002)), although its role in HER2 signaling is unclear.

ErbB2 belongs to the EGFR/ErbB receptor tyrosine kinase family, which also includes ErbB1/EGFR, ErbB3, and ErbB4. ErbB members can form homodimers or heterodimers depending on binding to specific ligand. Although no ligand for ErbB2 has been identified, ErbB2 acts as a co-receptor for ErbB1, ErbB3, and ErbB4 when the latter binds to EGF family members or neuregulin respectively. Upon ligand binding, ErbB members become dimerized and activated. Activated ErbBs phosphorylate various tyrosine residues in the cytoplasmic domain, resulting in the recruitment and activation of various downstream signaling cascades including ras/raf/MAPK, phosphatidylinositol-3 kinase (PI-3K), and tyrosine phosphatase SHP-2, which provide signals for various cellular responses including proliferation and survival. It is still not clear how each of the ErbB-activated cascades contribute to breast carcinogenesis in vivo. Nevertheless, since ErbB2 overexpression inducing breast tumor formation suggests that enhanced PTK activity or ErbB downstream signal relay/adapter components may contribute to breast carcinogenesis.

Although Gab-like molecules have not been implicated in breast cancer previously, overexpression of Gab1 in fibroblasts has been shown to potentiate EGF-mediated cell growth and transformation and anchorage independent growth, and Gab2 is important for cytokine-induced cell growth via its ability to activate PI-3K. Although Gab2 tyrosine phosphorylation and its associated PI-3K activity have been correlated with EGF-induced cell proliferation, the functional evolvement of Gab2 in EGF-mediated cell growth has not been fully investigated.

Overexpression of Gab2 may also play a role in neoplastic disorders. Although mutations in PTPN11 were not detected in a large panel of human breast tumors (Bentires-Alj, M. et al., Cancer Res. 64, 8816-8820 (2004)), studies described herein, as well as other previous studies, showed that Gab2 is overexpressed in breast cancer (Daly, R. J. et al., Oncogene 21, 5175-5181 (2002)). Furthermore, using fluorescence in situ hybridization (FISH) analysis, the Gab2 gene was located on human chromosome 11q13.5-14.1 (Yamada, K et al., Cytogenet. Cell Genet. 94, 39-42(2001)), which maps within a region (11q13) amplified in approximately 10-15% of human breast tumors (Ormnandy, C. J. et al., Breast Cancer Res. Treat. 78, 323-335 (2003)); Bekri, S. et al. Cytogenet. Cell Genet. 79, 125-131 (1997)). Because chromosome 11q13 amplification has been found in 10-15% of breast cancer patients, Gab2 expression in breast cancer cell lines and tumors was examined and Gab2 was found to be overexpressed in ˜40% of breast cancer cell lines and 20% of primary breast tumor samples tested. Whether Gab2 is amplified in breast tumors, the extent to which gene amplification contributes to Gab2 overexpression and whether overexpression of Gab2 has a causal role in mammary carcinogenesis have remained unknown.

Applicants have discovered at least two effects of Gab2 overexpression which can contribute to human breast carcinogenesis. Firstly, Gab2 overexpression alone can increase the proliferative capacity of mammary epithelial cells. Secondly, when overexpressed with Neu and, conceivably, the products of other oncogenes, Gab2 confers a more invasive phenotype in mice. Both effects reflect the ability of Gab2 to enhance activation of the Shp2-Erk pathway, and can be antagonized, at least ex vivo, by Mek inhibitors. Gab2 was also found to be amplified in human breast cancer, and Gab2 amplification maybe a major cause of Gab2 overexpression. Given the effects of Gab2 overexpression in three-dimensional culture and in transgenic mice, these results suggest that amplification of Gab2, in combination with other genetic abnormalities, may contribute to the genesis of breast tumors with 11q13-14 amplification.

In one embodiment, the invention encompasses Gab2, its homologs, analogs, variants, mutants, complementary nucleic acid sequences. Encompassed by the present invention are proteins that have substantially the same amino acid sequence as Gab2, or polynucleotides that have substantially the same nucleic acid sequence as the polynucleotides encoding Gab2. “Substantially the same sequence” means a nucleic acid or polypeptide that exhibits at least about 90% sequence identity with a reference sequence, e.g., another nucleic acid or polypeptide, preferably at least about 95% identity, and more preferably at least about 97% sequence identity with the reference sequence. The length of comparison for sequences will generally be at least 75 nucleotide bases or 25 amino acids, more preferably at least 150 nucleotide bases or 50 amino acids, and most preferably 243-264 nucleotide bases or 81-88 amino acids. “Polypeptide” as used herein indicates a molecular chain of amino acids and does not refer to a specific length of the product. Thus, peptides, oligopeptides and proteins are included within the definition of polypeptide. This term is also intended to include polypeptide that have been subjected to post-expression modifications such as, for example, glycosylations, acetylations, phosphorylations and the like.

“Sequence identity,” as used herein, refers to the subunit sequence similarity between two polymeric molecules, e.g., two polynucleotides or two polypeptides. When a subunit position in both of the two molecules is occupied by the same monomeric subunit, e.g., if a position in each of two peptides is occupied by serine, then they are identical at that position. The identity between two sequences is a direct function of the number of matching or identical positions, e.g, if half (e.g, 5 positions in a polymer 10 subunits in length) of the positions in two peptide or compound sequences are identical, then the two sequences are 50% identical; if 90% of the positions, e.g, 9 of 10 are matched, the two sequences share 90% sequence identity. By way of example, the amino acid sequences R₂R₅R₇R₁₀R₆R₃ and R₉R₈R₁R₁₀R₆R₃ have 3 of 6 positions in common, and therefore share 50% sequence identity, while the sequences R₂R₅R₇R₁₀R₆R₃ and R₈R₁R₁₀R₆R₃ have 3 of 5 positions in common, and therefore share 60% sequence identity. The identity between two sequences is a direct function of the number of matching or identical positions. Thus, if a portion of the reference sequence is deleted in a particular peptide, that deleted section is not counted for purposes of calculating sequence identity, e.g., R₂R₅R₇R₁₀R₆R₃ and R₂R₅R₇R₁₀R₃ have 5 out of 6 positions in common, and therefore share 83.3% sequence identity.

Identity is often measured using sequence analysis software e.g., BLASTN or BLASTP (available at http://www.ncbi.nlm.nih.gov/BLAST/). The default parameters for comparing two sequences (e.g., “Blast”-ing two sequences against each other, http://www.ncbi.nlm.nih.gov/gorf/b12.html) by BLASTN (for nucleotide sequences) are reward for match=1, penalty for mismatch=−2, open gap=5, extension gap=2. When using BLASTP for protein sequences, the default parameters are reward for match=0, penalty for mismatch=0, open gap=11, and extension gap=1.

When two sequences share “sequence homology,” it is meant that the two sequences differ from each other only by conservative substitutions. For polypeptide sequences, such conservative substitutions consist of substitution of one amino acid at a given position in the sequence for another amino acid of the same class (e.g., amino acids that share characteristics of hydrophobicity, charge, pK or other conformational or chemical properties, e.g., valine for leucine, arginine for lysine), or by one or more non-conservative amino acid substitutions, deletions, or insertions, located at positions of the sequence that do not alter the conformation or folding of the polypeptide to the extent that the biological activity of the polypeptide is destroyed. Examples of “conservative substitutions” include substitution of one non-polar (hydrophobic) residue such as isoleucine, valine, leucine or methionine for another, the substitution of one polar (hydrophilic) residue for another such as between arginine and lysine, between glutamine and asparagine, between threonine and serine; the substitution of one basic residue such as lysine, arginine or histidine for another, or the substitution of one acidic residue, such as aspartic acid or glutamic acid for another, or the use of a chemically derivatized residue in place of a non-derivatized residue; provided that the polypeptide displays the requisite biological activity. Two sequences which share sequence homology may called “sequence homologs.”

Homology, for polypeptides, is typically measured using sequence analysis software (e.g., Sequence Analysis Software Package of the Genetics Computer Group, University of Wisconsin Biotechnology Center, 1710 University Avenue, Madison, Wis. 53705). Protein analysis software matches similar sequences by assigning degrees of homology to various substitutions, deletions, and other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine.

The invention also contemplates mutants of the proteins and peptides disclosed herein, where the mutation(s) do not substantially alter the activity of the protein or peptide, that is the mutations are effectively “silent” mutations.

By “mutant” of Gab2 is meant a polypeptide that includes any change in the amino acid sequence relative to the amino acid sequence of the equivalent reference Gab2 polypeptide. Such changes can arise either spontaneously or by manipulations by man, by chemical energy (e.g., X-ray), or by other forms of chemical mutagenesis, or by genetic engineering, or as a result of mating or other forms of exchange of genetic information. Mutations include, e.g., base changes, deletions, insertions, inversions, translocations, or duplications. Mutant forms of Gab2 may display either increased or decreased activity relative to the equivalent reference Gab2 polynucleotide, and such mutants may or may not also comprise additional amino acids derived from the process of cloning, e.g, amino acid residues or amino acid sequences corresponding to full or partial linker sequences.

Mutants/fragments of the protein of the present invention can be generated by PCR cloning. To make such fragments, PCR primers are designed from known sequence in such a way that each set of primers will amplify known subsequence from the overall protein. These subsequences are then cloned into an appropriate expression vector and the expressed protein tested for its activity as described in the assays described herein.

Mutants/fragments of the protein of the present invention can also be generated by Pseudomonas elastase digestion, as described by Mariyama, M. et al. (1992, J. Biol. Chem. 267:1253-8).

By “analog” of Gab2 is meant a non-natural molecule substantially similar to either the entire Gab2 molecule or a fragment or allelic variant thereof, and having substantially the same or superior biological activity. Such analogs are intended to include derivatives (e.g., chemical derivatives, as defined above) of the biologically active Gab2, as well as its fragments, mutants, homologs, and allelic variants, which derivatives exhibit a qualitatively similar agonist or antagonist effect to that of the unmodified Gab2 polypeptide, fragment, mutant, homolog, or allelic variant.

By “allele” of Gab2 is meant a polypeptide sequence containing a naturally-occurring sequence variation relative to the polypeptide sequence of the reference Gab2 polypeptide. By “allele” of a polynucleotide encoding the Gab2 polypeptide is meant a polynucleotide containing a sequence variation relative to the reference polynucleotide sequence encoding the reference Gab2 polypeptide, where the allele of the polynucleotide encoding the Gab2 polypeptide encodes an allelic form of the Gab2 polypeptide.

It is possible that a given polypeptide may be either a fragment, a mutant, an analog, or allelic variant of Gab2, or it may be two or more of those things, e.g., a polypeptide may be both an analog and a mutant of the Gab2 polypeptide. For example, a shortened version of the Gab2 molecule (e.g., a fragment of Gab2) may be created in the laboratory. If that fragment is then mutated through means known in the art, a molecule is created that is both a fragment and a mutant of Gab2. In another example, a mutant may be created, which is later discovered to exist as an allelic form of Gab2 in some mammalian individuals. Such a mutant Gab2 molecule would therefore be both a mutant and an allelic variant. Such combinations of fragments, mutants, allelic variants, and analogs are intended to be encompassed in the present invention.

Also encompassed by the present invention are chemical derivatives of Gab2. “Chemical derivative” refers to a subject polypeptide having one or more residues chemically derivatized by reaction of a functional side group. Such derivatized residues include for example, those molecules in which free amino groups have been derivatized to form amine hydrochlorides, p-toluene sulfonyl groups, carbobenzoxy groups, t-butyloxycarbonyl groups, chloroacetyl groups or formyl groups. Free carboxyl groups may be derivatized to form salts, methyl and ethyl esters or other types of esters or hydrazides. Free hydroxyl groups may be derivatized to form O-acyl or O-alkyl derivatives. The imidazole nitrogen of histidine may be derivatized to form N-imbenzylhistidine. Also included as chemical derivatives are those peptides which contain one or more naturally occurring amino acid derivatives of the twenty standard amino acids. For examples: 4-hydroxyproline may be substituted for proline; 5-hydroxylysine may be substitute for lysine; 3-methylhistidine may be substituted for histidine; homoserine may be substituted for serine; and ornithine may be substituted for lysine.

The present invention also includes fusion proteins and chimeric proteins comprising the Gab2 protein, its fragments, mutants, homologs, analogs, and allelic variants. A fusion or chimeric protein can be produced as a result of recombinant expression and the cloning process, e.g, the protein may be produced comprising additional amino acids or amino acid sequences corresponding to full or partial linker sequences, comprises additional vector sequence added to the protein, including a HA tag. As used herein, the term “fusion or chimeric protein” is intended to encompass changes of this type to the original protein sequence. A fusion or chimeric protein can consist of a multimer of a single protein, e.g, repeats of the Gab2 protein, or the fusion and chimeric proteins can be made up of several proteins. The tern “fusion protein” or “chimeric protein” as used herein can also encompass additional components for e.g., delivering a chemotherapeutic agent, wherein a polynucleotide encoding the chemotherapeutic agent is linked to the polynucleotide encoding the Gab2 protein.

Multimeric proteins comprising Gab2, its fragments, mutants, homologs, analogs and allelic variants are also intended to be encompassed by the present invention. By “multimer” is meant a protein comprising two or more copies of a subunit protein. The subunit protein may be the protein of the present invention, e.g., Gab2 repeated two or more times, or a fragment, mutant, homolog, analog or allelic variant, e.g, a Gab2 mutant or fragment, repeated two or more times. Such a multimer may also be a fusion or chimeric protein, e.g, a repeated Gab2 mutant may be combined with polylinker sequence, and/or one or more peptides, e.g., Gab2-associated peptides, which may be present in a single copy, or may also be tandemly repeated, e.g, a protein may comprise two or more multimers within the overall protein.

In one embodiment, the invention encompasses Gab2, its homologs, analogs, variants, mutants, complementary nucleic acid sequences, and sequences which can be used to design probes which hybridize to Gab2 or its complementary strand. The design of the probes should preferably follow these parameters: (a) it should be designed to an area of the sequence which has the fewest ambiguous bases (“N's”), if any, and (b) it should be designed to have a Tₘ of approx. 80° C. (assuming 2° C. for each A or T and 4 degrees for each G or C).

The probes should preferably be labeled with g-³²P-ATP (specific activity 6000 Ci/mmole) and T4 polynucleotide kinase using commonly employed techniques for labeling oligonucleotides. Other labeling techniques can also be used. Unincorporated label should preferably be removed by gel filtration chromatography or other established methods. The amount of radioactivity incorporated into the probe should be quantitated by measurement in a scintillation counter. In one embodiment, specific activity of the resulting probe should be approximately 4×10⁶ dpm/pmole. The bacterial culture containing the pool of full-length clones can be thawed and 100 μl of the stock used to inoculate a sterile culture flask containing 25 ml of sterile L-broth containing ampicillin at 100 μg/ml The culture can be grown to saturation at 37° C., and the saturated culture should preferably be diluted in fresh L-broth. Aliquots of these dilutions can be plated to determine the dilution and volume which will yield approximately 5000 distinct and well-separated colonies on solid bacteriological media containing L-broth containing ampicillin at 100 μg/ml and agar at 1.5% in a 150 mm petri dish when grown overnight at 37° C. Other known methods of obtaining distinct, well-separated colonies can also be employed.

Standard colony hybridization procedures can then be used to transfer the colonies to nitrocellulose filters and lyse, denature and bake them. Highly stringent conditions include those that are at least as stringent as, for example, 1×SSC at 65° C., or 1×SSC and 50% formamide at 42° C. Moderate stringency conditions include those that are at least as stringent as, for example, 4×SSC at 65° C., or 4×SSC and 50% formamide at 42° C. Reduced stringency conditions can include, for example, those that are at least as stringent as 4×SSC at 50° C., or6×SSC and 50% formamide at 40° C.

The filter is then preferably incubated at 65° C. for 1 hour with gentle agitation in 6×SSC (20× stock is 175.3 g NaCl/liter, 88.2 g Na citrate/liter, adjusted to pH 7.0 with NaOH) containing 0.5% SDS, 100 μg/ml of yeast RNA, and 10 mM EDTA (approximately 10 mL per 150 mm filter). The probe can then added to the hybridizon mix at a concentration greater than or equal to 1×10⁶ dpm/mL. The filter is then preferably incubated at 65° C. with gentle agitation overnight. The filter is then preferably washed in 500 mL of 2×SSC/0.5% SDS at room temperature without agitation, preferably followed by 500 mL of 2×SSC/0.1% SDS at room temperature with gentle shaking for 15 minutes. A third wash with 0.1×SSC/0.5% SDS at 65° C. for 30 minutes to 1 hour is optional. The filter is then preferably dried and subjected to autoradiography for sufficient time to visualize the positives on the X-ray film. Other known hybridization methods can also be employed. The positive colonies are then picked, grown in culture, and plasmid DNA isolated using standard procedures. The clones can then be verified by restriction analysis, hybridization analysis, or DNA sequencing.

Stringency conditions for hybridization refers to conditions of temperature and buffer composition which permit hybridization of a first nucleic acid sequence to a second nucleic acid sequence, wherein the conditions determine the degree of identity between those sequences which hybridize to each other. Therefore, “high stringency conditions” are those conditions wherein only nucleic acid sequences which are very similar to each other will hybridize. The sequences may be less similar to each other if they hybridize under moderate stringency conditions. Still less similarity is needed for two sequences to hybridize under low stringency conditions. By varying the hybridization conditions from a stringency level at which no hybridization occurs, to a level at which hybridization is first observed, conditions can be determined at which a given sequence will hybridize to those sequences that are most similar to it. The precise conditions determining the stringency of a particular hybridization include not only the ionic strength, temperature, and the concentration of destabilizing agents such as formamide, but also on factors such as the length of the nucleic acid sequences, their base composition, the percent of mismatched base pairs between the two sequences, and the frequency of occurrence of subsets of the sequences (e.g., small stretches of repeats) within other non-identical sequences. Washing is the step in which conditions are set so as to determine a minimum level of similarity between the sequences hybridizing with each other. Generally, from the lowest temperature at which only homologous hybridization occurs, a 1% mismatch between two sequences results in a 1° C. decrease in the melting temperature (Tₘ) for any chosen SSC concentration. Generally, a doubling of the concentration of SSC results in an increase in the Tₘ of about 17° C. Using these guidelines, the washing temperature can be determined empirically, depending on the level of mismatch sought. Hybridization and wash conditions are explained in Current Protocols in Molecular Biology (Ausubel, F. M. et al., eds., John Wiley & Sons, Inc., 1995, with supplemental updates) on pages 2.10.1 to 2.10.16, and 6.3.1 to 6.3.6.

High stringency conditions can employ hybridization at either (1) 1×SSC (10×SSC=3 M NaCl, 0.3 M Na₃-citrate-2H₂O (88 g/liter), pH to 7.0 with 1 M HCl), 1% SDS (sodium dodecyl sulfate), 0.1-2 mg/ml denatured salmon sperm DNA at 65° C., (2) 1×SSC, 50% formamide, 1% SDS, 0.1-2 mg/ml denatured salmon sperm DNA at 42° C., (3) 1% bovine serum albumen (fraction V), 1 mM Na₂·EDTA, 0.5 M NaHPO₄ (pH 7.2) (1 M NaHPO₄=134 g Na₂HPO₄·7H₂O, 4 ml 85% H₃PO₄ per liter), 7% SDS, 0.1-2 mg/ml denatured salmon sperm DNA at 65° C., (4) 50% formamide, 5×SSC, 0.02 M Tris-HCl (pH 7.6), 1× Denhardt's solution (100×=10 g Ficoll 400, 10 g polyvinylpyrrolidone, 10 g bovine serum albumin (fraction V), water to 500 ml), 10% dextran sulfate, 1% SDS, 0.1-2 mg/ml denatured salmon sperm DNA at 42° C, (5) 5×SSC, 5× Denhardt's solution, 1% SDS, 100 μg/ml denatured salmon sperm DNA at 65° C., or (6) 5×SSC, 5× Denhardt's solution, 50% formamide, 1% SDS, 100 μg/ml denatured salmon sperm DNA at 42° C., with high stringency washes of either (1) 0.3-0.1×SSC, 0.1% SDS at 65° C, or (2) 1 mM Na₂EDTA, 40 mM NaHPO₄ (pH 7.2), 1% SDS at 65° C. The above conditions are intended to be used for DNA-DNA hybrids of 50 base pairs or longer. Where the hybrid is believed to be less than 18 base pairs in length, the hybridization and wash temperatures should be 5-10° C. below that of the calculated Tₘ of the hybrid, where Tₘ in ° C.=(2×the number of A and T bases)+(4×the number of G and C bases). For hybrids believed to be about 18 to about 49 base pairs in length, the Tₘ in ° C.=(81.5° C.+16.6 (log₁₀ M)+0.41 (% G+C)−0.61 (% formamide)−500/L), where “M” is the molarity of monovalent cations (e.g., Na⁺), and “L” is the length of the hybrid in base pairs.

Moderate stringency conditions can employ hybridization at either (1) 4×SSC, (10×SSC=3 M NaCl, 0.3 M Na₃-citrate-2H₂O (88 g/liter), pH to 7.0 with 1 M HCl), 1% SDS (sodium dodecyl sulfate), 0.1-2 mg/ml denatured salmon sperm DNA at 65° C., (2) 4×SSC, 50% formamide, 1% SDS, 0.1-2 mg/ml denatured salmon sperm DNA at 42° C., (3) 1% bovine serun albumen (fraction V), 1 mM Na₂·EDTA, 0.5 M NaHPO₄ (pH 7.2) (1 M NaHPO₄=134 g Na₂HPO₄·7H₂O, 4 ml 85% H₃PO₄ per liter), 7% SDS, 0.1-2 mg/ml denatured salmon sperm DNA at 65° C., (4) 50% formamide, 5×SSC, 0.02 M Tris-HCl (pH 7.6), 1× Denhardt's solution (100 ×=10 g Ficoll 400, 10 g polyvinylpyrrolidone, 10 g bovine serum albumin (faction V), water to 500 ml), 10% dextran sulfate, 1% SDS, 0.1-2 mg/ml denatured salmon sperm DNA at 42° C., (5) 5×SSC, 5× Denhardt's solution, 1% SDS, 100 μg/ml denatured salmon sperm DNA at 65° C., or (6) 5×SSC, 5× Denhardt's solution, 50% formamide, 1% SDS, 100 μg/ml denatured salmon sperm DNA at 42° C., with moderate stringency washes of 1×SSC, 0.1% SDS at 65° C. The above conditions are intended to be used for DNA-DNA hybrids of 50 base pairs or longer. Where the hybrid is believed to be less than 18 base pairs in length, the hybridization and wash tempers should be 5-10° C. below that of the calculated Tₘ of the hybrid, where Tₘ in ° C.=(2×the number of A and T bases) +(4 x the number of G and C bases). For hybrids believed to be about 18 to about 49 base pairs in length, the Tₘ in ° C.=(81.5° C.+16.6 (log₁₀ M)+0.41 (% G+C)−0.61 (% formamide)−500/L), where “M” is the molarity of monovalent cations (e.g, Na⁺), and “L” is the length of the hybrid in base pairs.

Low stringency conditions can employ hybridization at either (1) 4×SSC, (10×SSC=3 M NaCl, 0.3 M Na₃-citrate-2H₂O (88 g/liter), pH to 7.0 with 1 M HCl), 1% SDS (sodium dodecyl sulfate), 0.1-2 mg/ml denatured salmon sperm DNA at 50° C., (2) 6×SSC, 50% formamide, 1% SDS, 0.1-2 mg/ml denatured salmon sperm DNA at 40° C., (3) 1% bovine serum albumen (fraction V), 1 mM Na₂·EDTA, 0.5 M NaHPO₄ (pH 7.2) (1 M NaHPO₄=134 g Na₂HPO₄·7₂O, 4 ml 85% H₃PO₄ per liter), 7% SDS, 0.1-2 mg/ml denatured salmon sperm DNA at 50° C., (4) 50% formamide, 5×SSC, 0.02 M Tris-HCl (pH 7.6), 1× Denhardt's solution (100×=10 g Ficoll 400, 10 g polyvinylpyrrolidone, 10 g bovine serum albumin (fraction V), water to 500 ml), 10% dextran sulfate, 1% SDS, 0.1-2 mg/ml denatured salmon sperm DNA at 40° C., (5) 5×SSC, 5× Denhardt's solution, 1% SDS, 100 μg/ml denatured salmon sperm DNA at 50° C., or (6) 5×SSC, 5× Denhardt's solution, 50% formamide, 1% SDS, 100 μg/ml denatured salmon sperm DNA at 40° C., with low stringency washes of either 2×SSC, 0.1% SDS at 50° C., or (2) 0.5% bovine serum albumin (fraction V), 1 mM Na₂EDTA, 40 mM NaHPO₄ (pH 7.2), 5% SDS. The above conditions are intended to be used for DNA-DNA hybrids of 50 base pairs or longer. Where the hybrid is believed to be less than 18 base pairs in length, the hybridization and wash temperatures should be 5-10° C. below that of the calculated Tₘ of the hybrid, where Tₘ in ° C.=(2×the number of A and T bases)+(4×the number of G and C bases). For hybrids believed to be about 18 to about 49 base pairs in length, the Tₘ in ° C.=(81.5° C.+16.6 (log₁₀ M)+0.41 (% G+C)−0.61 (% formamide)−500/L), where “M” is the molarity of monovalent cations (e.g, Na⁺), and “L” is the length of the hybrid in base pairs.

The invention also encompasses the use of wild type or mutant versions of Gab2 as inserts for vectors. Such vectors can be used to produce Gab2 proteins in large quantities. They can also be used for the delivery of nucleic acids to a cell, e.g., a host cell. Such a vector may also bring about the replication and/or expression of the transferred nucleic acid pieces and can be used to produce Gab2 protein in large quantities. Examples of vectors include nucleic acid molecules derived, e.g., from a plasmid, bacteriophage, or a mammalian, plant or insect virus, or non-viral vectors such as ligand-nucleic acid conjugates, liposomes, or lipid-nucleic acid complexes. It may be desirable that the transferred nucleic molecule is operatively linked to an expression control sequence to form an expression vector capable of expressing the transferred nucleic acid. Such transfer of nucleic acids is generally called “transformation,” and refers to the insertion of an exogenous polynucleotide into a host cell, irrespective of the method used for the insertion. For example, direct uptake, transduction or f-mating are included. The exogenous polynucleotide may be maintained as a non-integrated vector, for example, a plasmid, or alternatively, may be integrated into the host genome. “Operably linked” refers to a situation wherein the components described are in a relationship permitting them to function in their intended manner, e.g., a control sequence “operably linked” to a coding sequence is ligated in such a manner that expression of the coding sequence is achieved under conditions compatible with the control sequence. A “coding sequence” is a polynucleotide sequence which is transcribed into mRNA and translated into a polypeptide when placed under the control of (e.g., operably linked to) appropriate regulatory sequences. The boundaries of the coding sequence are determined by a translation start codon at the 5′-terminus and a translation stop codon at the 3′-terminus. Such boundaries can be naturally-occurring, or can be introduced into or added the polynucleotide sequence by methods known in the art. A coding sequence can include, but is not limited to, mRNA, cDNA, and recombinant polynucleotide sequences.

The vector into which the cloned polynucleotide is cloned may be chosen because it functions in a prokaryotic, or alternatively, it is chosen because it functions in a eukaryotic organism. Two examples of vectors which allow for both the cloning of a polynucleotide encoding the Gab2 protein, and the expression of this protein from the polynucleotides, are the pET22b and pET28(a) vectors (Novagen, Madison, Wis., USA) and a modified pPICZαA vector (InVitrogen, San Diego, Calif., USA), which allow expression of the protein in bacteria and yeast, respectively. See for example, WO 99/29878, the entire teachings which are hereby incorporated by reference.

Once a polynucleotide has been cloned into a suitable vector, it can be transformed, or transfected, into an appropriate host cell. By “host cell” is meant a cell which has been or can be used as the recipient of transferred nucleic acid by means of a vector. Host cells can be prokaryotic or eukaryotic, mammalian, plant, or insect, and can exist as single cells, or as a collection, e.g, as a culture, or in a tissue culture, or in a tissue or an organism. Host cells can also be derived from normal or diseased tissue from a multicellular organism, e.g, a mammal. “Host cell”, as used herein, is intended to include not only the original cell which was transformed with a nucleic acid, but also descendants of such a cell, which still, comprise, or contain, the nucleic acid.

In one embodiment, the isolated polynucleotide encoding the Gab2 protein additionally comprises a polynucleotide linker encoding a peptide. Such linkers are known to those of skill in the art and, for example the linker can comprise at least one additional codon encoding at least one additional amino acid. Typically the linker comprises one to about twenty or thirty amino acids. The polynucleotide linker is translated, as is the polynucleotide encoding the Gab2 protein, resulting in the expression of a Gab2 protein with at least one additional amino acid residue at the amino or carboxyl terminus of the protein. Importantly, the additional amino acid, or amino acids, do not compromise the activity of the Gab2 protein.

After inserting the selected polynucleotide into the vector, the vector is transformed into an appropriate prokaryotic strain and the strain is cultured (e.g, maintained) under suitable culture conditions for the production of the biologically active Gab2 protein, thereby producing a biologically active Gab2protein, or mutant, derivative, fragment or fusion protein thereof. In one embodiment, the invention comprises cloning of a polynucleotide encoding a Gab2 protein into the vectors pET22b, pET17b or pET28a, which are then transformed into bacteria. The bacterial host strain then expresses the Gab2 protein.

In another embodiment of the present invention, the eukaryotic vector comprises a modified yeast vector. One method is to use a pPICzα plasmid wherein the plasmid contains a multiple cloning site. The multiple cloning site has inserted into it a HA.Tag motif. Additionally the vector can be modified to add a NdeI site, or other suitable restriction sites. Such sites are well known to those of skill in the art.

One method of producing Gab2, for example, is to amplify the polynucleotide of SEQ ID NO:6, and clone it into an expression vector, transform the vector containing the polynucleotide into a host cell capable of expressing the polypeptide encoded by the polynucleotide, culturing the transformed host cell under culture conditions suitable for expressing the protein, and then extracting and purifying the protein from the culture.

In another embodiment, the Gab2 protein may also be expressed as a product of transgenic animal, e.g., as a component of the milk of transgenic cows, goats, sheep or pigs, or as a product of a transgenic plant, e.g., combined or linked with starch molecules in maize. These methods can also be used with a subsequence of SEQ ID NO:6 to produce portions of the protein of SEQ ID NO:5.

Gab2 may also be produced by conventional, known methods of chemical synthesis. Methods for constructing the proteins of the present invention by synthetic means are known to those skilled in the art The synthetically-constructed Gab2 protein sequence, by virtue of sharing primary, secondary or tertiary structural and/or conformational characteristics with e.g., recombinantly-produced Gab2, may possess biological properties in common therewith, including biological activity. Thus, the synthetically-constructed Gab2 protein sequence may be employed as biologically active or immunological substitute for e.g., recombinantly-produced, purified Gab2 protein in screening of therapeutic compounds and in immunological processes for the development of antibodies.

Polynucleotides encoding Gab2 can be cloned out of isolated DNA or a cDNA library. Nucleic acids and polypeptides referred to herein as “isolated” are nucleic acids or polypeptides substantially free (i e., separated away from) the material of the biological source from which they were obtained (e.g., as exists in a mixture of nucleic acids or in cells), which may have undergone further processing. “Isolated” nucleic acids or polypeptides include nucleic acids or polypeptides obtained by methods described herein, similar methods, or other suitable methods, including essentially pure nucleic acids or polypeptides, nucleic acids or polypeptides produced by chemical synthesis, by combinations of chemical or biological methods, and recombinantly produced nucleic acids or polypeptides which are isolated. An isolated polypeptide therefore means one which is relatively free of other proteins, carbohydrates, lipids, and other cellular components with which it is normally associated. An isolated nucleic acid is not immediately contiguous with (i.e., covalently linked to) both of the nucleic acids with which it is immediately contiguous in the naturally-occurring genome of the organism from which the nucleic acid is derived. The term, therefore, includes, for example, a nucleic acid which is incorporated into a vector (e.g., an autonomously replicating virus or plasmid), or a nucleic acid which exists as a separate molecule independent of other nucleic acids such as a nucleic acid fragment produced by chemical means or restriction endonuclease treatment.

An extracellular stimulus is any substance (e.g., a compound such as a molecule) that associates or interacts with a cell, directly or indirectly, such that the association or interaction results in a signaling cascade within the cell. Such extracellular stimuli include cytokines, growth factors, hormones and antigens.

In another embodiment, the biological function of Gab2 can be compromised by inhibitors which can specially block the interaction of Gab2 with its associated molecules. Such inhibitors may be useful in the treatment of numerous disorders, including allergic responses, immunodisorders and cancer (e.g., breast cancer, ovarian cancer, leukemia). For example, Gab2 is constitutively tyrosine phosphorylated in a variety of cells transformed by BCR-ABL, the oncogene responsible for chronic myelogenous leukemia, as well as by its relative, the TEL-ABL fusion protein, suggesting that deregulation of a cascade involving SHP-2 and Gab2 may contribute to cell transformation. In addition, over-expression and/or constitutive phosphorylation of Gab2 could contribute to other diseases including other neoplastic diseases.

As used herein, the term “inhibitor of Gab2 interaction/function” refers to an agent (e.g., an oligonucleotide, a molecule, a compound, or a protein) which can inhibit or eliminate a (i.e., one or more) function of Gab2. Inhibition can be partial or complete. For example, an inhibitor of Gab2 function can inhibit or eliminate Gab2's association with one or more proteins (e.g., SHP-2, p85, Grb2, ERBB2/HER2/Neu (wild-type and oncogenic mutants thereof), NeuNT), inhibit or eliminate Gab2 expression, and/or inhibit or eliminate signal transduction mediated through Gab2 (e.g., MAPK activation, c-fos gene transcription, Erk activation).

SHP-2 promotes activation of the Ras-Erk pathway. As described herein (see Example 21), Gab2 promotes proliferation of human breast epithelial cells via a SHP-2/Erk signaling pathway. Notably, inhibiting Erk activation can abolish the effects of Gab2 overexpression on cell proliferation. Therefore, one or more Erk signaling pathway inhibitors can be used to inhibit the effects of Gab2 overexpression (e.g., increased cell proliferation). Suitable Erk signaling pathway inhibitors include, but are not limited to, inhibitors of Erk activation, for example, inhibitors comprising isolated RNA that mediates RNA interference against mRNA of a component of an Erk signaling pathway (e.g., SHP-2, Ras, Raf, Mek, Erk), inhibitors of Erk and kinases that act upstream of Erk (e.g. Ras inhibitors, Raf inhibitors, Mek inhibitors), several of which are commercially available, as well as lovostatin and other HMG CoA reductase inhibitors, and/or Farnesyl transferase inhibitors. Such inhibitors can be used in the prevention and/or treatment of disorders, injuries and diseases associated with Gab2 overexpression (e.g., breast cancer, ovarian cancer, leukemia). Accordingly, Gab2-mediated processes and cellular responses (e.g., proliferation, migration, chemotactic responses, secretion or degranulation (e.g, acute rejection, chronic rejection) ) can be inhibited with an inhibitor of Gab2 function. As used herein, “Gab2” refers to naturally occurring Gab2 (including vertebrate Gab2, e.g., mammalian Gab2, such as human (Homo sapiens) Gab2) and also encompasses naturally occurring variants, such as allelic variants and splice variants.

Preferably, the inhibitor of Gab2 function is a compound which is, for example, a small organic molecule, natural product, protein (e.g., antibody, cytokine, antigen), peptide or peptidomimetic. Inhibitors of Gab2 function can be identified, for example, by screening libraries or collections of molecules, such as, the Chemical Repository of the National Cancer Institute, as described herein or using other suitable methods.

The term “natural product”, as used herein, refers to a compound which can be found in nature, for example, naturally occurring metabolites of marine organisms (e.g., tunicates, algae), plants or other organisms and which possess biological activity, e.g., can inhibit Gab2 function.

Natural products can be isolated and identified by suitable means. For example, a suitable biological source (e.g., vegetation) can be homogenized (e.g., by grinding) in a suitable buffer and clarified by centrifugation, thereby producing an extract. The resulting extract can be assayed for the capacity to inhibit Gab2 function, for example, by the assays described herein. Extracts which contain an activity that inhibit Gab2 function can be further processed to isolate the Gab2 inhibitor by suitable methods, such as, fractionation (e.g., column chromatography (e.g., ion exchange, reverse phase, affinity), phase partitioning, fractional crystallization) and assaying for biological activity. Once isolated the structure of a natural product can be determined (e.g., by nuclear magnetic resonance (NMR)) and those of skill in the art can devise a synthetic scheme for synthesizing the natural product. Thus, a natural product can be isolated (e.g., substantially purified) from nature or can be fully or partially synthetic. A natural product can be modified (e.g., derivatized) to optimize its therapeutic potential. Thus, the term “natural product”, as used herein, includes those compounds which are produced using standard medicinal chemistry techniques to optimize the therapeutic potential of a compound which can be isolated from nature.

A “neoplastic disorder”, or “Cancer” means neoplastic growth, hyperplastic or proliferative growth or a pathological state of abnormal cellular development and includes solid tumors, non-solid tumors, and any abnormal cellular proliferation, such as that seen in leukemia. As used herein, “cancer” also means Gab2-dependent cancers and tumors, i.e., tumors that require for their growth (expansion in volume and/or mass) an amplification/overexpression of Gab2 (e.g., breast cancer, ovarian cancer, leukemia). “Regression” refers to the reduction of tumor mass and size as determined using methods well-known to those of skill in the art.

The term “neoplastic disease”, as used herein, refers to malignant pathologies involving a Gab2-mediated injury such as, but not limited to, malignancies involving Gab2, including breast cancer, prostate cancer, ovarian cancer, carcinomas, such as Ductal Carcinoma in situ, and leukemias (chronic myelocytic, chronic lymphocytic and/or myelodyspastic syndrome); and lymphomas (Hodgkin's and non-Hodgkin's lymphomas, such as malignant lymphomas (Burkitt's lymphoma or Mycosis fungoides)).

The term “allergic response”, as used herein, refers to inflammatory or allergic diseases and conditions, including, but not limited to, respiratory allergic diseases such as asthma, allergic rhinitis, hypersensitivity lung diseases, hypersensitivity pneumonitis, interstitial lung diseases (ILD) (e.g., idiopathic pulmonary fibrosis, or ILD associated with rheumatoid arthritis, systemic lupus erythematosus, ankylosing spondylitis, systemic sclerosis, Sjogren's syndrome, polymyositis or dermatomyositis); systemic anaphylaxis or hypersensitivity responses, drug allergies (e.g., to penicillin, cephalosporins), insect sting allergies; inflammatory bowel diseases, such as Crohn's disease and ulcerative colitis; spondyloarthropathies; scleroderma; psoriasis and inflammatory dermatoses such as dermatitis, eczema, atopic dermatitis, allergic contact dermatitis, urticaria; vasculitis (e.g., necrotizing, cutaneous, and hypersensitivity vasculitis).

The term “immune disorder”, as used herein, refers to any immune disease, including autoimmune diseases including, but not limited to, arthritis (e.g., rheumatoid arthritis, juvenile rheumatoid arthritis, psoriatic arthritis), multiple sclerosis, systemic lupus erythematosus, myasthenia gravis, juvenile onset diabetes, nephritides such as glomerulonephritis, autoimmune thyroiditis, Behcet's disease, graft rejection (e.g., in transplantation); or to other diseases or conditions (including Gab2-mediated diseases or conditions), in which undesirable inflammatory responses are to be inhibited can be treated, including, but not limited to, reperfusion injury, atherosclerosis, certain hematologic malignancies, cytokine-induced toxicity (e.g., septic shock, endotoxic shock), polymyositis, dermatomyositis.

The terms “prevent,” “preventing,” or “prevention,” as used herein, mean reducing the probability/likelihood or risk of contraction of a Gab2-mediated disease, disorder or injury by a subject, delaying the onset of a condition related to a Gab2-mediated disease, disorder or injury in a subject, lessening the severity of one or more symptoms of a Gab2-mediated disease, disorder or injury in a subject, or any combination thereof. In general, the subject of a preventative regimen most likely will be categorized as being “at-risk”, e.g., the risk for the subject contracting a Gab2-mediated disease, disorder or injury is higher than the risk for an individual represented by the relevant baseline population.

As used herein, the terms “treat,” “treating,” or “treatment,” mean to counteract a medical condition (e.g., a condition related to a Gab2-mediated disease, disorder or injury) to the extent that the medical condition is improved according to a clinically-acceptable standard. For example, “to treat a Gab2-mediated disease, disorder or injury” means to improve the ability of a subject to resist contraction of a Gab2-mediated disease, disorder or injury and/or to relieve symptoms of a condition related to a Gab2-mediated disease, disorder or injury in a subject, wherein the improvement and relief are evaluated using a clinically-acceptable standard. Treatment can be partial or complete and can include the alleviation of one or more symptoms of a Gab2-mediated disease, disorder or injury.

In another embodiment, the nucleic acid sequence of Gab2 of the invention or its homologs, fragments or complementary sequences can be used to design antisense oligonucleotides (e.g., antisense RNA, microRNA). In addition, naturally-occurring nucleic acid molecules (e.g., microRNA), that bind to and inhibit the expression of Gab2 mRNA, the mRNA of a Gab2-associated protein (e.g., SHP-2), and/or the mRNA of a component downstream of Gab2 in a Gab2-mediated signaling cascade (e.g., an Erk pathway), can also be usedSuch agents can be administered via a variety of routes, including nasal, systemic or topical. In one embodiment, they can be used in anti-allergy therapy.

The agent (e.g., Gab2 inhibitor, or additional therapeutic agent, for example, an Erk signaling pathway inhibitor, an inhibitor of an anti-apoptotic protein, an inhibitor of ERBB2/HER2/Neu signaling) can be administered by any suitable parenteral or nonparenteral route, including, for example, topically (e.g., cream, ointment), or nasally (e.g., solution, suspension). Parenteral administration can include, for example, intramuscular, intravenous, intraarticular, intraarterial, intrathecal, subcutaneous, or intraperitoneal administration The agent (e.g., Gab2 inhibitor, additional therapeutic agent) can also be administered orally (e.g., in capsules, suspensions, tablets or dietary), transdermally, topically, by inhalation (e.g., intrabronchial, intranasal, oral inhalation or intranasal drops) or rectally. Administration can be local or systemic as indicated. The preferred mode of administration can vary depending upon the particular agent (e.g., Gab2 inhibitor, additional therapeutic agent) chosen, however, oral, systemic or parenteral administration is generally preferred.

In some embodiments, one or more inhibitors or agents described herein can be administered sequentially or co-administered simultaneously. Delivery can be in vitro, in vivo, or ex vivo. Delivery can be via a variety of means, including transfection, transformation and electroporation. The cell can be present in a biological sample obtained from the patient (e.g., blood, bone marrow) and used in the treatment of disease such as cancer, immunosuppression/immunostimulation, neurodegeneration or cardiac hypertrophy, or can be obtained from cell culture and used to dissect cell proliferation, cell death or protein degradation cascades in in vivo and in vitro systems. After contact with the viral vector comprising Gab2 or a Gab2 mutant, the sample can be returned or readministered to a cell or patient according to methods known to those practiced in the art. In the case of delivery to a patient or experimental animal model (e.g., rat, mouse, monkey, chimpanzee), such a treatment procedure is sometimes referred to as ex vivo treatment or therapy. Frequently the cell is targeted from the patient or animal and returned to the patient or animal once contacted with the viral vector comprising the activated mutant of the present invention. Ex vivo gene therapy has been described, for example, in Kasid, et al., Proc. Natl. Acad. Sci. USA 87:473 (1990); Rosenberg, et al., New Engl. J. Med. 323:570 (1990); Williams, et al., Nature 310476 (1984); Dick, et al, Cell 42:71 (1985); Keller, et al., Nature 318:149 (1985) and Anderson, et al., U.S. Pat. No. 5,399,346 (1994).

Use of timed release or sustained release delivery systems are also included in the invention. Such systems are highly desirable in situations where surgery is difficult or impossible, e.g., patients debilitated by age or the disease course itself, or where the risk-benefit analysis dictates control over cure.

A sustained-release matrix, as used herein, is a matrix made of materials, usually polymers, which are degradable by enzymatic or acid/base hydrolysis or by dissolution. Once inserted into the body, the matrix is acted upon by enzymes and body fluids. The sustained-release matrix desirably is chosen from biocompatible materials such as liposomes, polylactides (polylactic acid), polyglycolide (polymer of glycolic acid), polylactide co-glycolide (co-polymers of lactic acid and glycolic acid) polyanhydrides, poly(ortho)esters, polyproteins, hyaluronic acid, collagen, chondroitin sulfate, carboxylic acids, fatty acids, phospholipids, polysaccharides, nucleic acids, polyamino acids, amino acids such as phenylalanine, tyrosine, isoleucine, polynucleotides, polyvinyl propylene, polyvinylpyrrolidone and silicone. A preferred biodegradable matrix is a matrix of one of either polylactide, polyglycolide, or polylactide co-glycolide (co-polymers of lactic acid and glycolic acid).

The agent (e.g., Gab2 inhibitor, additional therapeutic agent) can be administered as a neutral compound or as a salt or esther. Salts of compounds containing an amine or other basic group can be obtained, for example, by reacting with a suitable organic or inorganic acid, such as hydrogen chloride, hydrogen bromide, acetic acid, perchloric acid and the like. Compounds with a quaternary ammonium group also contain a counteranion such as chloride, bromide, iodide, acetate, perchlorate and the like. Salts of compounds containing a carboxylic acid or other acidic functional group can be prepared by reacting with a suitable base, for example, a hydroxide base. Salts of acidic functional groups contain a countercation such as sodium, potassium and the like.

As used herein, the terms “pharmaceutically acceptable,” “physiologically tolerable” and grammatical variations thereof as they refer to compositions, carriers, diluents and reagents, are used interchangeably and represent that the materials are capable of administration to or upon a mammal with a minimum of undesirable physiological effects such as nausea, dizziness, gastric upset and the like. The preparation of a pharmacological composition that contains active ingredients dissolved or dispersed therein is well understood in the art and need not be limited based on formulation. Typically such compositions are prepared as injectables either as liquid solutions or suspensions, however, solid forms suitable for solution, or suspensions, in liquid prior to use can also be prepared. The preparation can also be emulsified.

The inhibitor of Gab2 function can be administered to the individual as part of a pharmaceutical composition comprising a Gab2 inhibitor and a pharmaceutically or physiologically acceptable carrier. Pharmaceutical compositions for co-therapy can comprise an inhibitor of Gab2 function and one or more additional therapeutic agents. An inhibitor of Gab2 function and an additional therapeutic agent can be components of separate pharmaceutical compositions which can be mixed together prior to administration or administered separately. Formulation will vary according to the route of administration selected (e.g., solution, emulsion, capsule).

The active (e.g., therapeutic) ingredient can be mixed with excipients which are pharmaceutically acceptable and compatible with the active ingredient and in amounts suitable for use in the therapeutic methods described herein. Suitable pharmaceutical or physiological carriers can contain inert ingredients which do not interact with the inhibitor of Gab2 function and/or additional therapeutic agent. Standard pharmaceutical formulation techniques can be employed, such as those described in Remington's Pharmaceutical Sciences, Mack Publishing Company, Easton, Pa. Suitable carriers, e.g., excipients for parenteral administration, include, for example, sterile water, dextrose, glycerol, ethanol, physiological saline, bacteriostatic saline (saline containing about 0.9% benzyl alcohol), phosphate-buffered saline, Hank's solution, Ringer's-lactate and the like and combinations thereof. Methods for encapsulating compositions (such as in a coating of hard gelatin or cyclodextran) are known in the art (Baker, et al., “Controlled Release of Biological Active Agents”, John Wiley and Sons, 1986). In addition, if desired, the composition can contain minor amounts of auxiliary substances such as wetting or emulsifying agents, pH buffering agents and the like which enhance the effectiveness of the active ingredient.

As used herein, the term “effective amount” also means the total amount of each active component of the composition or method that is sufficient to show a meaningful patient benefit, i.e., treatment, healing, prevention or amelioration of the relevant medical condition, or an increase in rate of treatment, healing, prevention or amelioration of such conditions. For example, an “effective amount” of a Gab2 inhibitor is an amount sufficient to achieve a desired therapeutic and/or prophylactic effect, such as an amount sufficient to inhibit mast cell degranulation. In addition, an effective amount is an amount sufficient to inhibit a (i.e., one or more) function of Gab2 (e.g., Gab2 extracellular signal-induced c-fos activation, and/or Gab2 ligand-induced secretion (e.g. degranulation) of antihistamines), and thereby, inhibit a Gab2-mediated injury (e.g., allergic response, immune disorder, neoplastic disease). An “effective amount” of an additional therapeutic agent is an amount sufficient to achieve a desired therapeutic and/or prophylactic effect. When applied to a combination, the term refers to combined amounts of the active ingredients that result in the therapeutic effect, whether administered in combination, serially or simultaneously.

The amount of agent (e.g., Gab2 inhibitor, additional therapeutic agent) administered to the individual will depend on the characteristics of the individual, such as general health, age, sex, body weight and tolerance to drugs as well as the degree, seventy and type of rejection. The skilled artisan will be able to determine appropriate dosages depending on these and other factors.

The dosage of the agent of Gab2 of the present invention will also depend on the disease state or condition being treated along with the above clinical factors and the route of administration of the compound. For treating humans or animals, about 10 mg/kg of body weight to about 20 mg/kg of body weight of the protein can be administered. In combination therapies, e.g., the agents of the invention in combination with radiotherapy, chemotherapy, or immunotherapy, it may be possible to reduce the dosage, e.g., to about 0.1 mg/g of body weight to about 0.2 mg/kg of body weight. Depending upon the half-life of the agent in the particular animal or human, the agent can be administered between several times per day to once a week. It is to be understood that the present invention has application for both human and veterinary use. The methods of the present invention contemplate single as well as multiple administrations, given either simultaneously or over an extended period of time. In addition, the agent can be administered in conjunction with other forms of therapy, e.g, chemotherapy, radiotherapy, or immunotherapy.

The term “unit dose” when used in reference to a therapeutic composition of the present invention refers to physically discrete units suitable as unitary dosage for the subject, each unit containing a predetermined quantity of active material calculated to produce the desire therapeutic effect in association with the required diluent; i.e., carrier or vehicle. Preferred unit dosage formulations are those containing a daily dose or unit, daily sub-dose, or an appropriate fraction thereof of the administered ingredient. It should be understood that in addition to the ingredients, particularly mentioned above, the formulations of the present invention may include other agents conventional in the art having regard to the type of formulation in question. Optionally, cytotoxic agents may be incorporated or otherwise combined with the agent, or biologically functional protein fragments thereof, to provide dual therapy to the patient.

In addition, Gab2 nucleic acid sequences (e.g., cDNA sequence) can be used to identify relevant downstream targets of Gab2, for example, those required for allergic responses. Moreover, Gab2 nucleic acid sequences (e.g., cDNA sequence) along with critical binding molecule(s), can be used to screen for small molecule and/or natural product inhibitors of the interaction(s).

Conversely, defects in Gab2 expression and/or sequence may contribute to other diseases, such as immunodeficiency. Thus, the Gab2 coding sequence may also be useful for screening in various diseases, and in gene therapy applications.

In another embodiment, the novel scaffolding molecule Gab2 can be used to identify inhibitors of PH domain/lipids; Gab2/receptor (e.g., cytokine, growth factor, hormone and antigen receptor); Gab2/SH2 or SH3 protein interactions. Thus, inhibitors which can block Gab2 interaction with other intracellular molecules via PH domain, SH2, and SH3 domain can be potentially useful for immunosuppression and cancer therapy. Nucleic acid probes for Gab2 can also be used to detect upregulation or downregulation of Gab2 product in specimens from patients with leukemia such as CML, or to generate antibodies to detect changes in Gab2 expression or phosphorylation, for example, in patients with various neoplastic states.

RNA interference or “RNAi” is a term initially coined by Fire and co-workers to describe the observation that double-stranded RNA (dsRNA) can block gene expression when it is introduced into worms (Fire et al., Nature 391, 806-811 (1998)). dsRNA, including small interfering RNA (siRNA) and small hairpin RNA (shRNA), among others, directs target-specific (e.g., gene-specific), post-transcriptional silencing in many organisms, including vertebrates, and is a tool for studying gene function.

RNA interference can be used as a method for knocking down (partially or completely) (out) Gab2. This method of knocking out Gab2 gene expression can be used therapeutically or for research (e.g., to generate models of disease states, to examine the function of a Gab2, to assess whether an agent acts on a Gab2, to validate targets for drug discovery). In those instances in which Gab2 function is eliminated, the resulting cell or organism can also be referred to as a knockout. One embodiment of the method of producing knockdown cells and organisms comprises introducing into a cell or organism in which Gab2 is to be knocked down, RNA of sufficient length that targets Gab2 and maintaining the resulting cell or organism under conditions under which RNAi occurs, resulting in degradation of the mRNA of Gab2, thereby producing knockdown cells or organisms. Gab2 knockdown cells and organisms produced by the present method are also the subject of this invention.

Furthermore, RNAi can be used as a method of examining or assessing the function of Gab2 in a cell or organism. In one embodiment, RNA of sufficient length which targets mRNA of Gab2 is introduced into a cell or organism in which RNAi occurs. The cell or organism is referred to as a test cell or organism. The test cell or organism is maintained under conditions under which degradation of Gab2 mRNA occurs. The phenotype of the test cell or organism is then observed and compared to that of an appropriate control cell or organism, such as a corresponding cell or organism that is treated in the same manner except that Gab2 is not targeted. A difference between the phenotypes of the test and control cells or organisms provides information about the function of the degraded Gab2 mRNA. The information provided may be sufficient to identify (define) the function of Gab2 or may be used in conjunction with information obtained from other assays or analyses to do so.

Moreover, RNAi can be used as a method of validating whether an agent acts on Gab2. In this method, RNA of sufficient length that targets the Gab2 mRNA is introduced into a cell or organism in which RNAi occurs. Whether the agent has an effect on the cell or organism is determined.

In addition, RNAi can be used as a method of validating whether Gab2 is a target for drug discovery or development. RNA of sufficient length that targets Gab2 is introduced into a cell or organism. The cell or organism is maintained under conditions in which degradation of the Gab2 mRNA occurs, resulting in decreased expression of Gab2. Whether decreased expression of Gab2 has an effect on the cell or organism is determined, wherein if decreased expression of Gab2 has an effect, then the Gab2 product is a target for drug discovery or development.

RNAi can also be used as a method of treating a disease or condition associated with the presence of Gab2 protein in an individual comprising administering to the individual RNA of sufficient length which targets the mRNA of Gab2 (the mRNA that encodes the protein) for degradation. As a result, the protein is not produced or is not produced to the extent it would be in the absence of the treatment. Techniques for using such methods are found in PCT Application Number PCT/US01/10188 (WO 01/75164), the contents of which are incorporated by reference herein in their entirety.

The invention also encompasses genetically manipulated cell and animals, including knockout and transgenic mice, such as Gab2−/− mice or mice which overexpress Gab2 such as Whey Acidic Promoter-Gab2 (MMTV-Gab2) transgene mice as described herein.

In another embodiment, vectors described herein can be useful in a gene therapy setting, whereby a polynucleotide encoding the Gab2 protein, integrins, integrin subunits, or a mutant, fragment, or fusion protein thereof; is introduced and regulated in a patient. Various methods of transferring or delivering DNA to cells for expression of the gene product protein, otherwise referred to as gene therapy, are disclosed in Gene Transfer into Mammalian Somatic Cells in vivo, N. Yang, Crit. Rev. Biotechn. 12(4):335-356 (1992), which is hereby incorporated in its entirety by reference. Gene therapy encompasses incorporation of DNA sequences into somatic cells or germ line cells for use in either ex vivo or in vivo therapy. Gene therapy functions to replace genes, augment or inhibit normal or abnormal gene function, and to combat infectious diseases and other pathologies.

Strategies for treating these medical problems with gene therapy include therapeutic strategies such as identifying the defective gene and then adding a functional gene to either replace or inhibit the function of the defective gene or to augment a slightly functional gene; or prophylactic strategies, such as adding a gene for the product protein that will treat the condition or that will make the tissue or organ more susceptible to a treatment regimen.

Many protocols for transfer of the DNA or regulatory sequences of the Gab2 protein are envisioned in this invention. Transfection of promoter sequences, other than one normally found specifically associated with the Gab2 protein, or other sequences which would increase production of the Gab2 protein are also envisioned as methods of gene therapy.

Gene transfer methods for gene therapy fall into three broad categories: physical (e.g., electroporation, direct gene transfer and particle bombardment), chemical (e.g., lipid-based carriers, or other non-viral vectors) and biological (e.g, virus-derived vector and receptor uptake). For example, non-viral vectors may be used which include liposomes coated with DNA. Such liposome/DNA complexes may be directly injected intravenously into the patient. It is believed that the liposome/DNA complexes are concentrated in the liver where they deliver the DNA to macrophages and Kupffer cells. These cells are long lived and thus provide long term expression of the delivered DNA. Additionally, vectors or the “naked” DNA of the gene may be directly injected into the desired organ, tissue or tumor for targeted delivery of the therapeutic DNA.

Gene therapy methodologies can also be described by delivery site. Fundamental ways to deliver genes include ex vivo gene transfer, in vivo gene transfer, and in vitro gene transfer. In ex vivo gene transfer, cells are taken from the patient and grown in cell culture. The DNA is transfected into the cells, the transfected cells are expanded in number and then reimplanted in the patient. In in vitro gene transfer, the transformed cells are cells growing in culture, such as tissue culture cells, and not particular cells from a particular patient. These “laboratory cells” are transfected, the transfected cells are selected and expanded for either implantation into a patient or for other uses.

In vivo gene transfer involves introducing the DNA into the cells of the patient when the cells are within the patient. Methods include using virally mediated gene transfer using a noninfectious virus to deliver the gene in the patient or injecting naked DNA into a site in the patient and the DNA is taken up by a percentage of cells in which the gene product protein is expressed. Additionally, the other methods described herein, such as use of a “gene gun,” may be used for in vitro insertion of the DNA or regulatory sequences controlling production of the Gab2 protein.

Chemical methods of gene therapy may involve a lipid based compound, not necessarily a liposome, to transfer the DNA across the cell membrane. Lipofectins or cytofectins, lipid-based positive ions that bind to negatively charged DNA, make a complex that can cross the cell membrane and provide the DNA into the interior of the cell. Another chemical method uses receptor-based endocytosis, which involves binding a specific ligand to a cell surface receptor and enveloping and transporting it across the cell membrane. The ligand binds to the DNA and the whole complex is transported into the cell. The ligand gene complex is injected into the blood stream and then target cells that have the receptor will specifically bind the ligand and transport the ligand-DNA complex into the cell.

Many gene therapy methodologies employ viral vectors to insert genes into cells. For example, altered retrovirus vectors have been used in ex vivo methods to introduce genes into peripheral and tumor-infiltrating lymphocytes, hepatocytes, epidermal cells, myocytes, or other somatic cells. These altered cells are then introduced into the patient to provide the gene product from the inserted DNA.

Viral vectors have also been used to insert genes into cells using in vivo protocols. To direct the tissue-specific expression of foreign genes, WAP-acting regulatory elements or promoters that are known to be tissue-specific can be used. Alternatively, this can be achieved using in situ delivery of DNA or viral vectors to specific anatomical sites in vivo. For example, gene transfer to blood vessels in vivo was achieved by implanting in vitro transduced endothelial cells in chosen sites on arterial walls. The virus infected surrounding cells which also expressed the gene product. A viral vector can be delivered directly to the in vivo site, by a catheter for example, thus allowing only certain areas to be infected by the virus, and providing long-term, site specific gene expression. In vivo gene transfer using retrovirus vectors has also been demonstrated mammary tissue and hepatic tissue by injection of the altered virus into blood vessels leading to the organs.

Viral vectors that have been used for gene therapy protocols include but are not limited to, retrovirus, other RNA viruses such as poliovirus or Sindbis virus, adenovirus, adeno-associated virus, herpes viruses, SV 40, vaccinia and other DNA viruses. Replication-defective murine retroviral vectors are the most widely utilized gene transfer vectors. Murine leukemia retroviruses are composed of a single strand RNA complexed with a nuclear core protein and polymerase (pol) enzymes, encased by a protein core (gag) and surrounded by a glycoprotein envelope (env) that determines host range. The genomic structure of retroviruses include the gag, pol, and env genes enclosed at by the 5′ and 3′ long terminal repeats (LTR). Retroviral vector systems exploit the fact that a minimal vector containing the 5′ and 3′ LTRs and the packaging signal are sufficient to allow vector packaging, infection and integration into target cells providing that the viral structural proteins are supplied in trans in the packaging cell line. Fundamental advantages of retroviral vectors for gene transfer include efficient infection and gene expression in most cell types, precise single copy vector integration into target cell chromosomal DNA, and ease of manipulation of the retroviral genome.

The adenovirus is composed of linear, double stranded DNA complexed with core proteins and surrounded with capsid proteins. Advances in molecular virology have led to the ability to exploit the biology of these organisms to create vectors capable of transducing novel genetic sequences into target cells in vivo. Adenoviral-based vectors will express gene product proteins at high levels. Adenoviral vectors have high efficiencies of infectivity, even with low titers of virus. Additionally, the virus is fully infective as a cell free virion so injection of producer cell lines is not necessary. Another potential advantage to adenoviral vectors is the ability to achieve long term expression of heterologous genes in vivo.

Mechanical methods of DNA delivery include fusogenic lipid vesicles such as liposomes or other vesicles for membrane fusion, lipid particles of DNA incorporating cationic lipid such as lipofectin, polylysine-mediated transfer of DNA, direct injection of DNA, such as microinjection of DNA into germ or somatic cells, pneumatically delivered DNA-coated particles, such as the gold particles used in a “gene gun,” and inorganic chemical approaches such as calcium phosphate transfection. Particle-mediated gene transfer methods were first used in transforming plant tissue. With a particle bombardment device, or “gene gun,” a motive force is generated to accelerate DNA-coated high density particles (such as gold or tungsten) to a high velocity that allows penetration of the target organs, tissues or cells. Particle bombardment can be used in in vitro systems, or with ex vivo or in vivo techniques to introduce DNA into cells, tissues or organs. Another method, ligand-mediated gene therapy, involves complexing the DNA with specific ligands to form ligand-DNA conjugates, to direct the DNA to a specific cell or tissue.

It has been found that injecting plasmid DNA into muscle cells yields high percentage of the cells which are transfected and have sustained expression of marker genes. The DNA of the plasmid may or may not integrate into the genome of the cells. Non-integration of the transfected DNA would allow the transfection and expression of gene product proteins in terminally differentiated, non-proliferative tissues for a prolonged period of time without fear of mutational insertions, deletions, or alterations in the cellular or mitochondrial genome. Long-term, but not necessarily permanent, transfer of therapeutic genes into specific cells may provide treatments for genetic diseases or for prophylactic use. The DNA could be reinjected periodically to maintain the gene product level without mutations occurring in the genomes of the recipient cells. Non-integration of exogenous DNAs may allow for the presence of several different exogenous DNA constructs within one cell with all of the constructs expressing various gene products.

Electroporation for gene transfer uses an electrical current to make cells or tissues susceptible to electroporation-mediated mediated gene transfer. A brief electric impulse with a given field strength is used to increase the permeability of a membrane in such a way that DNA molecules can penetrate into the cells. This technique can be used in in vitro systems, or with ex vivo or in vivo techniques to introduce DNA into cells, tissues or organs.

Carrier mediated gene transfer in vivo can be used to transfect foreign DNA into cells. The carrier-DNA complex can be conveniently introduced into body fluids or the bloodstream and then site-specifically directed to the target organ or tissue in the body. Both liposomes and polycations, such as polylysine, lipofectins or cytofectins, can be used. Liposomes can be developed which are cell specific or organ specific and thus the foreign DNA carried by the liposome will be taken up by target cells. Injection of immunoliposomes that are targeted to a specific receptor on certain cells can be used as a convenient method of inserting the DNA into the cells bearing the receptor. Another carrier system that has been used is the asialoglycoportein/polylysine conjugate system for carrying DNA to hepatocytes for in vivo gene transfer.

The transfected DNA may also be complexed with other kinds of carriers so that the DNA is carried to the recipient cell and then resides in the cytoplasm or in the nucleoplasm. DNA can be coupled to carrier nuclear proteins in specifically engineered vesicle complexes and carried directly into the nucleus.

Gene regulation of the Gab2 may be accomplished by administering compounds that bind to the Gab2 gene, or control regions associated with the Gab2 gene, or its corresponding RNA transcript to modify the rate of transcription or translation. Additionally, cells transfected with a DNA sequence encoding the Gab2 protein may be administered to a patient to provide an in vivo source of those proteins. For example, cells may be transfected with a vector containing a nucleic acid sequence encoding the Gab2 protein. The transfected cells may be cells derived from the patient's normal tissue, the patient's diseased tissue, or may be non-patient cells.

For example, tumor cells removed from a patient can be transfected with a vector capable of expressing the agent of the present invention, and re-introduced into the patient. The transfected tumor cells produce levels of the agent in the patient that inhibit Gab2 function, thereby, inhibit the growth of the tumor. Patients may be human or non-human animals. Cells may also be transfected by non-vector, or physical or chemical methods known in the art such as electroporation, ionoporation, or via a “gene gun.” Additionally, the DNA may be directly injected, without the aid of a carrier, into a patient. In particular, the DNA may be injected into skin, muscle or blood.

The gene therapy protocol for transfecting the Gab2 agent into a patient may either be through integration of the Gab2 DNA into the genome of the cells, into minichromosomes or as a separate replicating or non-replicating DNA construct in the cytoplasm or nucleoplasm of the cell. Expression of the Gab2 protein may continue for a long-period of time or may be reinjected periodically to maintain a desired level of the protein(s) in the cell, the tissue or organ or a determined blood level.

In one embodiment, the Gab2 peptides of the present invention, as well as Gab2 fusion proteins, can be used to raise antibodies against, e.g., are specific for, Gab2. Such antibodies are referred to herein as “Gab2-specific antibodies”. Such peptides can be used to immunize or vaccinate an animal. Thus, the invention encompasses antibodies and antisera, which can be used for testing of novel Gab2 proteins, and can also be used in diagnosis, prognosis, prevention or treatment of diseases and conditions characterized by, or associated with, Gab2 activity or lack thereof.

Such antibodies and antisera can be combined with pharmaceutically-acceptable compositions and carriers to form diagnostic, prognostic or therapeutic compositions. The term “antibody” or “antibody molecule” refers to a population of immunoglobulin molecules and/or immunologically active portions of immunoglobulin molecules, i.e., molecules that contain an antibody combining site or paratope.

The antibodies of the present invention can be either polyclonal antibodies or monoclonal antibodies and can be raised against all or part of a Gab2 protein. Therefore, the antibodies of the present invention include peptide antibodies. In a particular embodiment, the peptide antibodies are peptide antibodies that recognize and bind to a SHP-2 binding site on a Gab2 protein. These antibodies that specifically bind to the Gab2 proteins or with a protein that associates with Gab2 can be used in diagnostic methods and kits that are well known to those of ordinary skill in the art to detect or quantify the Gab2 proteins in a body fluid or tissue. Results from these tests can be used to diagnose or predict the occurrence or recurrence of a cancer (e.g., breast cancer, ovarian cancer, leukemia) and other Gab2-mediated diseases.

The invention also includes use of the Gab2 protein, antibodies to this protein, and compositions comprising this protein and/or its antibodies in diagnosis or prognosis of diseases characterized by Gab2 activity. As used herein, the term “prognostic method” means a method that enables a prediction regarding the progression of a disease of a human or animal, e.g., a mammal diagnosed with the disease, in particular, an Gab2-dependent disease. The term “diagnostic method” as used herein means a method that enables a determination of the presence or type of Gab2-dependent disease in or on a human or animal.

Accordingly, the invention also encompasses methods and kits for the diagnosis and prognosis of Gab2-dependent diseases and disorders. Examples of Gab2-dependent diseases and disorders which may be diagnosed or prognosed using the methods and kits of the invention include, but are not limited to, diseases or disorders resulting from altered expression (e.g., overexpression, underexpression) of a Gab2 gene product (e.g., a Gab2 RNA transcript, a Gab2 protein) in a cell (e.g., a cell from a subject having a Gab2-dependent disease or disorder). Suitable diseases or disorders resulting from altered expression of a Gab2 gene product include an allergic response, a neoplastic disease or disorder and an immune disorder, among others.

In various embodiments, the diagnostic and prognostic methods comprise detecting the expression of a Gab2 gene product in a sample from a subject. In a particular embodiment, the subject is a human. An increase or decrease in the expression of a Gab2 gene product in a sample from the subject, relative to the expression of the Gab2 gene product in a suitable control sample, is indicative of the subject having or being at risk for developing a disease or disorder associated with altered Gab2 expression. In one embodiment, the Gab2 gene product with altered expression is a Gab2 protein. Suitable techniques for detecting the expression of a protein in a biological sample are well known to those of skill in the art and include, but are not limited to, immunohistochemical staining (IHC), Western blot analysis, enzyme-linked immunosorbent assay (ELISA) and radioimmunoassays (RIA) using an antibody that specifically recognizes a protein of interest.(e.g., a Gab2-specific antibody).

In another embodiment, the Gab2 gene product with altered expression is a nucleic acid transcript of a Gab2 gene (e.g., Gab2 mRNA). Suitable techniques for detecting the expression of a nucleic acid transcript in a biological sample are well known in the art and include, but are not limited to, RT-PCR (e.g., quantitative, semi-quantitative), in situ hybridization (e.g., fluorescence in situ hybridization), microarray analysis and Northern blot analysis.

As described herein, a significant percentage of human breast cancers are associated with overexpression of Gab2 protein. Therefore, the invention encompasses a method for diagnosing whether a subject has or is at risk for developing a disorder associated with Gab2 overexpression, comprising detecting the expression of Gab2 protein in a sample from the subject. According to this method, an increase in the expression of Gab2 protein in the sample from the subject, relative to the expression of Gab2 protein in a suitable control sample, is indicative of the subject having or being at risk for developing a disorder associated with Gab2 overexpression. In a particular embodiment, the disorder associated with Gab2 overexpression is breast cancer. In a further embodiment, the breast cancer is associated with a Gab2 gene amplification. In another embodiment, the breast cancer is associated with ERBB2/HER24/Neu overexpression or the expression of a ERBB2/HER2/Neu oncogenic mutant protein.

Overexpression of a Gab2 gene product can result from amplification of the Gab2 gene. Therefore, the invention also provides a method for diagnosing whether a subject has or is at risk for developing a disorder associated with Gab2 overexpression, comprising testing one or more cells from the subject for the presence of a Gab2 gene amplification. The presence of a Gab2 gene amplification is indicative of the subject having or being at risk for developing a disorder associated with Gab2 overexpression. In a particular embodiment, the disorder associated with Gab2 overexpression is breast cancer. In a further embodiment, the breast cancer is associated with a Gab2 gene amplification. In another embodiment, the breast cancer is associated with ERBB2/HER2/Neu overexpression or the expression of a ERBB2/HER2/Neu oncogenic mutant protein.

A Gab2 gene amplification can be tested, for example, by determining the Gab2 gene copy number in one or more cells from the subject. In a certain embodiment, a Gab2 gene copy number that is greater than 2 is indicative of the presence of a Gab2 gene amplification. In a related embodiment, a Gab2 gene copy number that is at least two-fold greater in cells that are affected with the disorder, relative to normal control cells from the subject, is indicative of the presence of a Gab2 gene amplification.

Techniques for detecting the copy number of a gene are well known in the art and include, for example, in situ hybridization (e.g., fluorescence in situ hybridization (FISH) and Southern blotting techniques, as well as sequencing of genomic DNA. In one embodiment, the Gab2 gene copy number in one or more cells from a subject is determined by performing in situ hybridization using a nucleic acid probe comprising a Gab2-specific nucleotide sequence. In a particular embodiment, the nucleic acid probe comprising a Gab2-specific nucleotide sequence is BAC clone RP11-1149C10. As described herein, Gab2 nucleic acid sequences be used to design and produce suitable probes for in situ hybridization that hybridize to Gab2 or its complementary strand, according to standard techniques.

The invention also provides kits for the diagnosis and/or prognosis of Gab2-dependent diseases and disorders. Accordingly, the invention encompasses a kit for diagnosing a disorder associated with altered Gab2 expression. In a particular embodiment, the disorder associated with altered Gab2 expression is breast cancer that is associated with Gab2 overexpression. Suitable components for the kit of the invention include, but are not limited to, Gab2-specific antibodies for detecting the expression of Gab2 protein in a sample and nucleic acid probes (e.g., DNA probes, RNA probes) that comprise a Gab2-specific nucleotide sequence for detecting the level of a Gab2 gene transcript in a sample. In one embodiment, the diagnostic kit comprises a Gab2-specific antibody. As described herein, Gab2 peptides of the present invention can be used to raise antibodies against, and are specific for, Gab2. In another embodiment, the diagnostic kit comprises a nucleic acid probe that comprises a Gab2-specific nucleotide sequence. As described herein, Gab2 nucleic acid sequences be used to design and produce probes that hybridize to Gab2 or its complementary strand, according to standard techniques. In yet another embodiment, the diagnostic kit comprises both a Gab2-specific antibody and a nucleic acid probe that comprises a Gab2-specific nucleotide sequence.

In a certain embodiment, the diagnostic kit comprises a Gab2-specific antibody and/or nucleic acid probe comprising a Gab2-specific nucleotide sequence, which further comprise one or more detectable labels. As used herein, a “detectable label” refers to any moiety that is capable of being specifically detected (e.g., by a partner moiety), either directly or indirectly, and therefore, can be used to identify and/or isolate a molecule (e.g., an antibody, a nucleic acid probe) that comprises the detectable label. Suitable detectable labels include, but are not limited to, chromophores, fluorophores, haptens, radionuclides (e.g., ³²P, ³³P, ³⁵S), fluorescence quenchers, enzymes, enzyme substrates, affinity tags (e.g., biotin, avidin, streptavidin, etc.), mass tags, electrophoretic tags and epitope tags that are recognized by an antibody. In a particular embodiment, the detectable label is a fluorescent label (e.g., a fluorophore).

The Gab2 protein can be used in a diagnostic method and kit to detect and quantity antibodies capable of binding the protein. These kits would permit detection of circulating antibodies to the Gab2 protein. Patients that have such circulating anti-Gab2 protein antibodies may be more likely to have Gab2-related disorders, such as cancers, immune disorders or allergic disorders and may be more likely to have recurrences of cancer after treatments or periods of remission

The present invention is further illustrated by the following examples, which are not intended to be limiting in any way.

### EXAMPLES

### Example 1

### Purification and Sequencing of Gab2

Cell Culture

Cell lines were grown in RPMI/10% FCS and the appropriate cytoline/growth factors.

Northern Blotting

Blots containing mouse tissue poly(A) RNA (Clontech, Palo Alto, Calif.) or total RNA (10 μg) from murine hematopoietic cells (provided by Dr. D. Zhang, Beth Israel Deaconess Medical Center, Boston, Mass.) were hybridized to radiolabelled cDNA probes, as indicated.

Purification and Sequencing

Gab2 was purified from ˜5×10¹⁰P210BCR-ABL BaF3 cells. Affinity-purified rabbit anti-SHP-2 antibodies (2.6 mg) were crosslinked onto protein A Sepharose beads (Harlow, E. and Lane, D., Antibodies: A Laboratory Manual (Cold Spring Harbor, New York: Cold Spring Harbor Laboratory) (1988)) using dimethylpimelimidate (Pierce, Rockford, Ill.). P210BCR-ABL BaF3 cells (4×10⁹) were resuspended in 40 ml hypotonic buffer (HB) containing protease and phosphatase inhibitors (Timms, J. F. et al., Mol. Cell. Biol. 18, 3838-3850 (1998)) and homogenized. Lysates clarified at 100,000× g were loaded onto Q-Sepharose and washed successively with HB and 20 mM Tris, pH 7.4/100 mM NaCl. Bound material was eluted in 20 mM Tris, pH 7.4/350 mM NaCl, diluted to a solution of 20 mM Tris 7.4 /150 mM NaCl/0.2% NP40, incubated with the anti-SHP-2 antibody beads, and washed with five volumes each of 20 mM Tris, pH 7.4/500 mM NaCl and 100 mM glycine, pH 6.0. Bound material was eluted in 100 mM glycine, pH 2.5, and neutralized with 1M Tris, pH 8.0. Pooled eluates from 10 cycles of this protocol were concentrated (Centricon-30; Amicon, Bedford, Mass.), acetone-precipitated, and resolved by SDS-PAGE.

A fraction of the final preparation was transferred to a nylon membrane and stained for total protein (Amersham, Piscataway, N.J.) and with anti-phosphotyrosine antibodies (anti-pTyr), and the immunoblots were examined. In addition to SHP-2 and Gab2, co-purifying species included an 85 kDa band, which was identified as the p85 subunit of PI-3K, and a band at ˜150 kDa (p150). The 150 kDa species associated with SHP-2 only in some BCR-ABL-transformed hematopoietic cell lines (Gu, H. et al., J. Biol. Chem. 272, 16421-16430 (1997)). Although SHP-2 was reported to associate with P210BCR-ABL (Tauchi, T. et al., J. Biol. Chem. 269, 15381-7 (1994)), a ˜210 kDa species was barely detectable in the final preparation, indicating that it is an association of low stoichiometry.

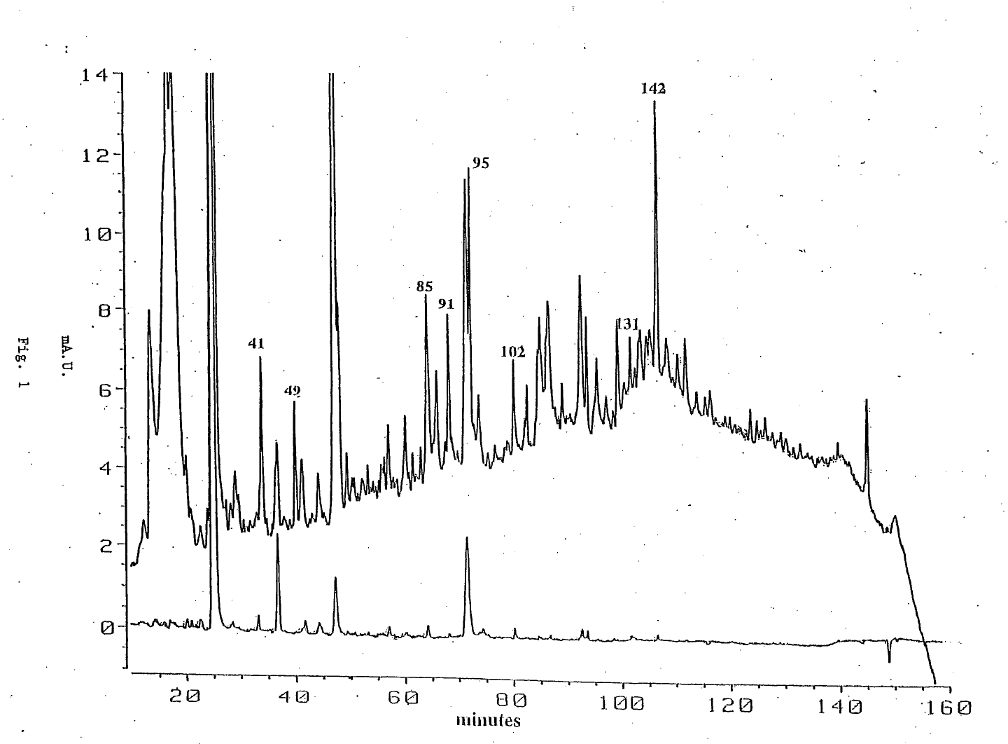

The 97 kDa band (which represented Gab2) was digested with endoprotease Lys-C. The resultant peptides were resolved by reverse-phase HPLC. The HPLC-resolved Lys-C peptides (nine peaks) were sequenced by Edman degradation (FIG. 1), with additional support from MS/MS sequencing, by the Harvard Microchemistry Facility. These peptides (Table 1) did not match any protein in the database, indicating that the 97 kDa band was novel.

TABLE 1
List of peptides obtained by Edman sequencing

| | | |
|---|---|---|
| Peptide No. | Sequence | Location in p97 |

| | | |
|---|---|---|
| GK102 | (S/G/A) [G][G]* | 2-4 |
| GK124 | [Q]LEED[Y][G][L][S](K)(G) | not present |
| PK85 | TQALQN[T]-(Q)* | 641-649 |
| PK91 | [D][S]TYDLPR[S]LA* | 260-270 |
| GK41 | (Q/E/P)(I/S)(L/R)(D/H)(N/K)TEFK | 251-259 |
| GK49 | VD = VQVDK | 631-638 |
| GT131 | ELQDSFVFDIK | 81-91 |
| GT142 | AKPTPLDLRNNTVIDEL* | 512-529 |
| GK95 | SSLTGSETDNEDVYTFK | 277-293 |

Note:
[ ]Amino acids were detennined with reasonable probability.
( )Amino acids were detemilned with low probability.
The amino acids which are present in the Gab2 protein are underlined.
*these peptides are incomplete sequences.

### Example 2

### Cloning of Gab2

Reverse transcription-polymerase chain reaction (RT-PCR) was used to obtain a cDNA fragment corresponding to peptide GT142, and this fragment was used to clone full length Gab2 cDNAs. Degenerate primers corresponding to all possible codons for the sequences KAKPTP (SEQ ID NO: 1) and TVIDEL (SEQ ID NO: 2) in peptide GT-142 (Table 1) were synthesized and used in a RT-PCR reaction with total RNA from P210BCR-ABL BaF3 cells. The expected 68 bp PCR product was subcloned into pUC19. Three inserts were sequenced and found to encode GT-142. A unique sequence (5′CCTTGACCTGAGAAACAACAC3′) (SEQ ID NO: 3) encoding the middle of GT-142 (LDLRNN)(SEQ ID NO: 4) served as the 5′primer in a 3′RACE reaction (GIBCO-BRL, Rockville, Md.), yielding a single 800 bp product. Its sequence revealed a single open reading frame containing peptides GT142, GK49 and PK85 (FIG. 2; Table 1). The 800 bp product was used as to probe a BaF3 cell cDNA library in λUniZap (provided by Dr. Alan D'Andrea, Dana Farber Cancer Institute, Boston, Mass.). Twenty positive clones were obtained, the two largest containing ˜5 kb inserts. Plasmids containing cDNAs were recovered by superinfection, according to the manufacturer's instructions (Stratagene, La Jolla, Calif.).

The two largest clones contained the consensus Kozak sequence specifying transnational initiation (GAC ATG AGC), an in-frame upstream stop codon, and a single long (1998 bp) open reading frame. The sequence predicted a 666 amino acid protein with a calculated molecular weight of 73 kDa (FIG. 2). The nucleotide sequence is deposited as GenBank Accession Number AF104244 (FIG. 6). Eight of nine Lys-C peptides obtained by microsequencing were found within the predicted sequence (FIG. 2 and Table 1), strongly suggesting that this cDNA encodes Gab2. Presumably, the one missing peptide derived from a trace contaminant.

This cDNA must encode bona fide Gab2 from BaF3 cells, because peptide GK102, comprising the p97 N-terminus, is predicted by this cDNA sequence, although it is absent in the sequence of unknown gene KIAA0571 (FIG. 3 and Table 1). KIAA0571 was deposited in GenBank (Accession Number AB011143) as part of a collection of human brain cDNAs (Nagase et al., DNA Res. 5, 31-39 (1998)). The KIAA0571 cDNA encoded a protein with 88% amino acid identity to Gab2, suggesting that KIAA0571 may be the human homolog of Gab2. The nucleotide sequences of the 5′ ends of Gab2 and KIAA0571 are highly divergent, such that the first 39 amino acids of Gab2, which comprises part of the PH domain, were absent in KIAA0571. In the absence of this sequence information, the likely function(s) of Gab2 cannot be deduced. In addition, the function of KIAA0571 open reading frame, were it to be expressed in vivo, is almost certain to be compromised, since PH domains bind to phospholipids and promote targeting to cellular membranes. KIAA0571 may represent an alternatively spliced form of Gab2, although cloning artifacts in KIAA0571 cannot be excluded.

The Gab2 protein contained an N-terminal PH domain that has a potential Grb2 SH2 domain binding site, followed by a long region with multiple potential tyrosyl phosphorylation sites, two serine-rich stretches (a.a. 126-188 and 532-600, respectively), and 2 PXXP sites, potentially capable of binding SH3 or WW domains (FIGS. 2, 3 and Table 2). Mammalian Gab1 and Drosophila Dos have similar topography and some sequence similarity to Gab2 (FIGS. 3, 4). The greatest similarity resides in their PH domains, with 73% identity between Gab2 and Gab1, and 45% identity between Gab2 and Dos (FIG. 4; top panel). Most of the potential phosphorylation sites and their relative positions within each protein are conserved, as is one of the PXXP motifs (FIG. 3 and Table 2).

TABLE 2
List of potential SH2 domain binding motifs in Gab2

| Potential Tyrosyl | SH2 Domain |
|---|---|
| Phosphorylation Sites | Containing proteins |
| YKNE | Grb2 |
| YSLP | PLCγ |
| YDLP | Crk or Nck |
| YQIP | Crk or PLCγ |
| YEYP | Crk |
| YVPM | p85 (PI3-K) |
| YIPM | p85 (PI3-K) |
| YVPM | p85 (PI3-K) |
| YLAL | SHP-2 |
| YVQV | SHP-2 |

There are also potentially significant sequence differences between these proteins. The Met binding domain (MBD) of Gab1 (a.a. 450-532) mediates association with c-Met/HGFR (Weidner et al., Nature 384, 173-176 (1996)). The cognate region in Gab2 (a.a. 443-514) exhibits only 36% amino acid identity (FIG. 4, bottom panel). The MBD, of Gab1, but not Gab2, contains two proline-rich stretches that comprise potential Grb2 SH3 domain-binding sites (Yu, H. et al., Cell 76, 933-945 (1994)). Conversely, two potential 14-3-3 binding sites (Yaffe, M. B. et al., Cell 91, 961-971 (1997)) are present only in Gab2 (RKS₁₆₀SAP, and RQS₆₅₈SEP). It remains to be determined whether Gab2 binds 14-3-3 proteins in vivo.

### Example 3

### Confirmation of Gab2 cDNA

To confirm that the cDNA encoded Gab2, a vector directing expression of HA-tagged Gab2 construct (Gab2HA) was transiently transfected into BaF3 cells. BaF3 cells were washed in serum-free RPMI, resuspended at 10⁷ cells/0.5 ml in RPMI/10% FCS, and incubated (10 minutes) with the indicated amounts of Gab2 expression vector and/or 20 μg of the SHP2 expression vector, the indicated amount of promoter luciferase reporter, and 20 ng of Renilla luciferase-TK reporter (Promega, Madison, Wis.).

Constructs encoding Gab2 (Gab2HA), Gab2 lacking amino acids 604-662 (Gab2ΔY2HA), and Gab2 with tyrosines 604/633 mutated to phenylalanine (Gab2DM), all with C-terminal HA tags, were constructed by PCR, using Gab2 cDNA as the template. PCR products were cloned into pEBB (from Dr. B. Mayer, Children's Hospital, Boston, Mass.), which directed expression under the control of the elongation factor 1-α promoter. The transfected Gab2HA fusion protein, as well as endogenous Gab2, migrated at an apparent mobility of about 97 kDa. Upon IL-3 stimulation, Gab2HA became tyrosyl phosphorylated and co-immunoprecipitated with SHP-2, consistent with its expected properties (Gu, H. et al., J. Biol. Chem. 272, 16421-16430 (1997)).

Further evidence was provided by studies with specific antibodies. A fragment encoding Gab2 a.a. 523-666was subcloned in frame into pGEX4T-1 (Pharmacia, Piscataway, N.J.). GST fusion protein was produced as described (Lechleider et al., J. Biol. Chem. 268, 13434-13438 (1993)). Affinity purified antibodies were prepared by passing antisera sequentially over GST and GST-Gab2 bound to Affi-Gel 15 (Bio-Rad, Hercules, Calif.), prepared as described (Frangioni et al., Cell 68, 545-560 (1992)). 4 μg of affinity purified antibodies quantitatively deplete Gab2 from 10⁷ BaF3 cells. Rabbit antibodies against peptide GT142 coupled to KLH were generated by BABCO (Berkeley, Calif.).

Immunoprecipitations and Immunoblotting.

Cell lysis, immunoprecipitation, immunoblotting, and detection by enhanced chemi-luminescence (Amersham, Piscataway, N.J.) were performed (Timms et al., Mol. Cell. Biol. 18, 3838-3850 (1998)). Monoclonal antibody 9E10 (against the myc-epitope) was obtained from BABCO Berkeley, Calif.). Monoclonal anti-phosphotyrosine antibody 4G10 was obtained from UBI (Santa Cruz). Anti-SHP2 immunoprecipitations utilized 1 μg antibody/10⁷ BaF3 cell equivalents. Anti-Gab2 immunoprecipitations utilized 4 μg antibody/10⁷ BaF3 cell equivalents. Dilutions for immunoblotting were: anti-SHP-2 (1:2500); anti-Grb2 (1:1000, Santa Cruz), anti-Shc (1:1,000, Transduction Laboratories, Lexington, Ky.); anti-p85 (1:3500, from Dr. C. Carpenter, Beth Israel Deaconess Medical Center); anti-MAPK (1:5,000, from Dr. J. Blenis, Harvard Medical School, Boston, Mass.); anti-Gab2 peptide antibodies (1:500); anti-GST-Gab2 antibodies (1:2500) and anti-pTyr antibodies (0.5 μg/ml).

BaF3 cells, starved in RPMI/0.8% BSA for 6 hours, were stimulated with recombinant IL-3 (10 ng/ml). Polyclonal antibodies against peptide GT-142 (Table 1) detected a 97 kDa protein in SHP-2 immunoprecipitates from IL-3-stimulated BaF3 cells; this protein co-migrated with the 97 kDa phosphotyrosyl protein associated with SHP-2. Antibodies against GST-Gab2 specifically immunoprecipitated 97 kDa and 70 kDa tyrosyl phosphorylated proteins from IL-3-stimulated BaF3 cells; these proteins co-migrated with the tyrosyl phosphoproteins present in anti-SHP-2 immunoprecipitates. Probing these immunoblots with anti-GST-p97 antibodies confirmed that the 97 kDa protein immunoprecipitated with anti-Gab2 and anti-SHP-2 antibodies is Gab2.

These data established that Gab2 is a 97 kDa SHP-2 binding protein. To determine whether Gab2 is the only component of the 97 kDa tyrosyl phosphorylated band associated with SHP-2 in IL-3-stimulated BaF3 cells, immunodepletion studies were performed. Quantitative depletion of Gab2 left no remaining 97 kDa phosphotyrosyl protein(s) in SHP-2 immunoprecipitates. Likewise, nearly all of the Gab2 in BaF3 cells associated with SHP-2 upon IL-3 stimulation (as indicated by a comparison of intensities of 97 kDa band in anti-Gab2 and anti-SHP-2 lanes), suggesting that SHP-2 probably is critical for signals emanating from Gab2. In contrast only ˜10% of SHP-2 was found in a complex with Gab2 upon IL-3 stimulation. This excess of SHP-2 is consistent with the possibility that it interacts with additional targets in BaF3 cells.

### Example 4

### Gab2 Expression and Response to Diverse Hematopoietic Stimuli

Since a 97 kDa protein was only observed associated with SHP-2 in hematopoietic cells, Gab2 was expected to be hematopoietic cell-specific. Indeed, Gab2 was expressed in many (but not all) hematopoietic cell lines, representing multiple lineages, and was not expressed in fibroblasts. Surprisingly, however, a 6 kb Gab2 transcript was observed in most tissues, with two additional smaller transcripts found in testis. Expression of Gab2 was highest in heart, testis, and lung, with lower levels in brain and liver. Although Gab2 participates in lymphocyte signaling, its expression was relatively low in spleen and thymus. Gab1 expression in the same tissues was largely overlapping, suggesting that Gab1 and Gab2 are co-expressed in at least some cell types. The relative levels of Gab2 and Gab1 expression are not the same in all tissues (as indicated by a comparison expression in brain, liver, and testis).

SHP-2 associated with proteins of similar size to Gab2 (95-110 kDa) in many signaling cascades, so Gab2 tyrosyl phosphorylation in response to a range of stimuli was examined. Kit225 cells (from Dr. P. Brennan, ICRF, London, U.K.), starved in RPMI/10% FCS for 48 hours, were stimulated with IL-2 (25 units/ml). BAC1.2F5 cells were stimulated as described (Timms et al., Mol. Cell. Biol. 18, 3838-3850 (1998)). WEHI 231 cells were stimulated with 15 μg/ml goat F(ab)′₂ anti-mouse IgG (Jackson ImmunoResearch Laboratories, West Grove, Pa.). Jurkat cells were stimulated with 1 μg/ml anti-CD3 antibody, OKT3 (ATCC), and crosslinked with 10 μg/ml rabbit anti-mouse IgG.

SHP-2 associated with a 100 kDa p-Tyr protein upon CSF-1 stimulation of myeloid progenitor cell lines (Carlberg and Rohrschneider, J. Biol. Chem. 272, 15943-15940 (1997)) or macrophages (Timms et al., Mol. Cell. Biol. 18, 3838-3850 (1998)). CSF-1 stimulation of BAC1.2F5 cells resulted in the rapid tyrosyl phosphorylation of Gab2 and its association with SHP-2. Similar results were obtained following IL-2 stimulation of Kit 225 cells or erythropoietin stimulation of erythropoietin receptor-expressing BaF3 cells. In Jurkat cells, TCR stimulation results in association of SHP-2 with a “110 kDa” p-Tyr protein (Frearson et al., Eur. J. Immunol. 26, 1539-1543 (1996); Frearson and Alexander, J. Exp. Med. 187, 1417-1426 (1998)); in these cells, Gab2 was rapidly tyrosyl phosphorylated and associated with SHP-2. B cell receptor (BCR) stimulation of WEHI-231 cells also led to Gab2 phosphorylation.

### Example 5

### Association of Gab2 with Other Signaling Molecules

The ability of Gab2 to also associate with other signaling molecules was examined. Tyrosyl phosphorylation of Gab2HA occurred within 1 minute of IL-3 stimulation of BaF3 cells, and then increased further, peaking by 10 minutes and accompanied by a dramatic decrease in Gab2 electrophoretic mobility. Besides SHP-2, tyrosyl phosphorylated Gab2 associated with Shc and the p85 subunit of PI-3K. In contrast, consistent with its PXXP motifs, Gab2 associated constitutively with Grb2. Shc, p85 and Grb2 also co-immunoprecipitated with endogenous Gab2 from IL-3-stimulated BaF3 cells. Sites within Gab2 conform to the consensus for the SH2 domains of Crk (and Crk-II and Crk-L) and PLCγ (FIG. 3 and Table 2), but using available immunoreagents, endogenous Gab2 or Gab2HA association with these proteins was not detected. Although Grb2 binds Gab2, Sos was not detected in Gab2 immunoprecipitates, perhaps because Grb2 binds primarily via its SH3 domain to Gab2.

### Example 6

### Role of Gab2 and the Gab2/SHP-2 Interaction in IL-3 Signaling

The effects of expressing wild type Gab2 and Gab2 mutants unable to bind SHP-2 on IL-3 signaling were examined. Two tyrosines within Gab2 (Y₆₀₄LAL/Y₆₃₃VQV) conform to the consensus for binding the SH2 domains of SHP-2 (Songyang et al., Cell 72, 767-778 (1993)); both sites are highly phosphorylated. A deletion mutant lacking these residues (a. a. 604-666) was generated, and a C-terminal HA tag was appended, as described in Example 5. This mutant (Gab2ΔY2HA) or wild type Gab2HA was transiently transfected into BaF3 cells.

Although Gab2ΔY2HA was expressed, it did not bind SHP-2 upon IL-3 stimulation IL-3-induced tyrosyl phosphorylation of Gab2ΔY2HA was similar, or even slightly less than that of wild type Gab2HA. The minimal effect on Gab2 tyrosyl phosphorylation observed upon mutating its SHP-2 binding sites contrasts with its increased tyrosyl phosphorylation upon over-expression of catalytically inactive mutants of SHP-2. Although C-terminal deletion eliminated SHP-2 binding, Gab2 association with p85 and Shc was not decreased, suggesting that Gab2 structure was not grossly perturbed in Gab2ΔY2HA. Grb2 binding to Gab2ΔY2HA also deceased slightly, particularly upon IL-3 stimulation, most likely because some Grb2 associates indirectly with Gab2 via binding to tyrosyl phosphorylated SHP-2 (Welham et al., J. Biol. Chem. 269, 23764-23768 (1994)).

SHP-2 is required for induction of c-fos. Therefore, the role of Gab2 in IL-3-induced c-fos promoter activity was assessed. For c-fos reporter assays, 1.5 μg of c-fos promoter (nt −710 to +42)-luciferase reporter were used (Hu et al., Science 268, 100-102 (1995)). STAT-driven transctivation was measured using 1.5 μg of a GAS-luciferase reporter (Jaster et al., Mol. Cell. Biol. 3364-3372 (1997)). For Elk assays, Gal4-Elk-1 (2 μg) and Gal4-luciferase (1 μg) were used (Bennett et al., Mol. Cell. Biol. 16, 1189-1202 (1996)). Cells were electroporated at 300V/800 μF, transferred to fresh RPMI/10% WEHI supernatant/10% FCS and, after three hours, were starved in RPMI/10% FCS. Twelve hours post-transfection, transfected cells (approximately 10⁶/condition) were incubated in RPMI/10% FCS alone or with murine IL-3 (1 ng/ml) for two hours. Luciferase assays were performed with a kit (Promega, Madison, Wis.). Promoter activities were normalized to Renilla luciferase levels.

Over-expression of wild type Gab2HA had little effect on IL-3-stimulated c-fos luciferase activity, although it did evoke a small (˜2-fold), but reproducible, increase in basal activity (FIG. 5A). However, expression of a comparable level of Gab2ΔY2HA decreased IL-3-evoked c-fos reporter activity (FIG. 5A) in a dose-dependent manner (FIG. 5B). Presumably, Gab2ΔY2HA displaced endogenous Gab2 from its proper location in vivo but, unable to bind SHP-2, Gab2ΔY2HA cannot transmit a signal necessary for full activation of c-fos. Dominant negative SHP-2 (SHP2ΔP) inhibited IL-3 induced c-fos activation (FIG. 5A), and the increased basal c-fos luciferase activity evoked by Gab2HA was blocked by dominant negative SHP-2. A Gab2 mutant in which Y604 and Y633 were converted to phenylalanine (Gab2DM) also lacked SHP-2 binding and inhibited IL-3-evoked c-fos luciferase activity (FIG. 5C). These findings suggest that Gab2 function, and, in particular, Gab2 binding to SHP-2, are required for full cytokine-induced c-fos activation.

The SRE (serum response element), which binds the SRF/TCF (serum response factor/TCF) complex is required for c-fos activation. MAPK phosphorylates Ets family transcription factors that comprise TCF, (e.g., Elk-1), increasing their transactivation potential. The ability of a Gal4-Elk-1 fusion to drive GAL4-luciferase activity in response to IL-3 was inhibited by Gab2ΔY2HA, indicating Gab2/SHP-2 was required for Elk-driven transactivation. STAT 5 also contributed to activation of the c-fos promoter in response to IL-3/GM-CSF (Rajotte et al., Blood 88, 2906-2916 (1996)). The Gab2/SHP-2 complex was required for full activation of a STAT-responsive element as well. Thus, Gab2/SHP-2 association was required for full cytokine-induced activation of the two major elements in the c-fos promoter.

The MEK inhibitor PD98059 ablated IL-3-induced c-fos luciferase activity, indicating that MEK/MAPK activation is essential for IL-3-induced c-fos promoter activation in BaF3 cells. Gab2ΔY2HA was expected to also inhibit IL-3-induced MAPK activity, particularly since SHP-2 is required for cytokine-induced MAPK activation. Transient over-expression of wild type Gab2HA potentiated MAPK activation in response to IL-3, suggesting that Gab2 can signal to MAPK. Surprisingly, however, Gab2ΔY2HA or Gab2DM not only failed to inhibit, but potentiated IL-3-evoked MAPK activation.

Because of these surprising findings, the role of SHP-2 in IL-3-induced MAPK activation was re-examined. For MAPK assays, BaF3 cells (10⁷) were co-transfected with 20 μg Gab2 or SHP2 expression plasmids and 2 μg of myc-tagged Erk1. Cells (5×10⁶) were stimulated with murine IL-3 for various times, washed, lysed in NP40 buffer, and Myc-Erk was immunoprecipitated with 9E10 (1 μl of ascites/sample). Kinase assays using myelin basic protein (MBP) were performed as described (Bennett et al., Mol. Cell. Biol. 16, 1189-1202 (1996)).

As mentioned above, previous work showed that SHP-2 is necessary for IL-3-induced MAPK activity. Consistent with these reports, two mutants of SHP-2, SHP2ΔP and SHP2CS inhibited IL-3-induced MAPK activation. It was concluded that the Gab2/SHP-2 complex was required for full activity of the c-fos promoter, acting on Ets and STAT family transcription factors via a cascade parallel to MAPK activation, and that SHP-2 must act at more than one point in cytokine signaling

### Example 7

### Characteristics of Gab2−/− BMMCs

Wild type (WT) and Gab2 knockout (−/−) BMMCs were incubated with 2 ug/ml anti-DNP monoclonal IgE (SPE-7) for 1 hour on ice. Cells were washed and incubated with FITC-anti-IgE rat monoclonal antibody for 30 minutes. Cell surface expression of FcεRI were determined by FACS, and found to be normal in Gab2−/− BMMCs.

BMMCs were sensitized with anti-DNP IgE, and stimulated with 10 ng/ml anti-dinitrophenol (DNP). Cells were lysed, and total cell lysates were resolved by SDS-PAGE, western blotted with anti-phosphotyrosine antibody (pTyr), and reprobed with anti-Akt antibodies. Total tyrosyl phosphorylation was found to be normal in Gab2−/− BMMCs upon FcεRI engagement.

BMMCs were sensitized with anti-DNP-IgE, stimulated with 10 ng/ml DNP, and lysed. Syk and LAT were immunoprecipitated by anti-Syk and LAT antibodies, resolved by SDS-PAGE, and western blotted with anti-phosphotyrosine antibody (pTyr), and reprobed with Syk antibodies. Syk and LAT tyrosyl phosphorylation are normal in Gab2−/− BMMCs upon FcεRI engagement.

BMMCs were sensitized with DNP mouse IgE (2 ug/ml) for 12 hours and labeled with ³H-serotonin for 3 hours. Unincorporated label was washed away and cells were stimulated for 15 minutes with the indicated concentrations of DNP. Serotonin released into the media and remaining in the cell pellet was quantified by scintillation counting, and it was determined that FcεRI-mediated degranulation is impaired in Gab2−/− BMMCs.

BMMCs were sensitized with anti-DNP-IgE, and stimulated with 10 ng/ml DNP for 0 and 1 hour. Total RNAs were isolated from BMMCs, and reverse-transcribed into first cDNA by reverse-transcriptase. The relative level of TNFα and IL-6 cDNAs in each sample was determined by Real time PCR, and Fcε-evoked TNFα and IL-6 gene expression are impaired in Gab2−/− BMMCs.

BMMCs were sensitized with anti-DNP-IgE, stimulated with 10 ng/ml DNP, and lysed. PLCγl was immunoprecipitated by anti-PLCγl antibodies, resolved by SDS-PAGE, and western blotted with anti-phosphotyrosine antibody (pTyr), and reprobed with anti-PLCγl antibodies. It was determined that tyrosyl phosphorylation of PLCγ is impaired in Gab2−/− BMMCs upon FcεRI crosslinking.

BMMCs were sensitized with anti-DNP IgE, and stimulated with 10 ng/ml DNP. Cells were lysed, and total cell lysates were resolved by SDS-PAGE, western blotted with anti-phospho-Akt antibodies, and reprobed with anti-Akt antibodies. Akt phosphorylation was found to be impaired in Gab2−/− mast cells upon FcεRI engagement.

BMMCs were sensitized with anti-DNP IgE, and stimulated with 10 ng/ml DNP. Cells were lysed, and total cell lysates were resolved by SDS-PAGE, western blotted with anti-phospho-Akt, phospho-p38, and phospho-MAPK antibodies, and reprobed with anti-Akt and MAPK antibodies. JNK and p38 phosphorylation is defective in Gab2−/− BMMCs, however, MAPK phosphorylation is not affected in Gab2−/− BMMCs.

Gab2−/− BMMCs were infected with MSCV-IRES-GFP virus expressing wild type Gab2 (WT) or virus alone (Vector). GFP positive BMMCs were sorted out by FACs, expanded, sensitized by IgE, and stimulated with 10 ng/ml DNP-HSA. Cells were lysed, and equal amount of total cell lysates were resolved by SDS-PAGE, transferred, and blotted with anti-phospho-Akt (473), phopho-JNK, phospho-38, and phospho-MAPK respectively. The same blot was reprobed with anti-Akt and Erk2. Expression of wild type Gab2 rescues signaling defects in Gab2−/− BMMCs.

20 ng of anti-DNP-IgE was injected intradermally into one ear of the mice. 24 hours later, 100 μg DNP in 200 μl of PBS with 2% Evans Blue was injected into the tail vein of the mice. 30 minutes later, the mice were sacrificed, and the ears were removed, cut into small pieces. Evans blue dye was extracted from the ears with formamide and 80° C. incubation for 3 hours, and quantified by reading OD at 610 nm. Passive cutaneous anaphylaxis was found to be defective in Gab2−/− mice.

### Example 8

### Location of Gab2 Gene on Human Chromosome 11 q13.3-14.2

Gab2 was purified and cloned from BCR-ABL transformed cells (Gu, H. et al., Mol Cell. 2, 729-740 (1998)). Unpublished data indicated that Gab2 can be phosphorylated by BCR-ABL in vitro. To further explore potential Gab2 involvement in human disease, FISH analysis was performed to localize the Gab2 gene on human genome. The Gab2 gene was located on human chromosome 11 q13.3-14. Importantly, chromosome 11 q13 is amplified in about 15% of primary breast cancers (Hui, R. et al., Oncogene 15, 1617-1623 (1997)). Cyclin D1, also located in the 11 q13 amplicon, is one of the few genes expressing in the breast tumor bearing this amplicon (Siegel, P. et al., Bioessays 22, 554-563 (2000)). The Gab2 gene may be amplified and expressed in breast tumors with the 11 q13 amplicon.

### Example 9

### Overexpression of Gab2 Protein in Breast Cancer Cell Lines and Breast Tumor Cells

The expression of Gab2 in breast cancer cell lines and breast tumor samples was examined by western blot analysis. The Gab2 protein was overexpressed in ˜40% breast cancer cell lines (16 total) examined, such as MDA-MB-134, 468, BT-20, T47D, MDA-MB-435, 21NT. In contrast Gab2 protein is just above detectable level in immortalized normal human mammary epithelia cell lines MCF-10A and 184B5. Furthermore, Gab2 protein level was high in ˜20% breast tumor samples (total 30 samples) compared to normal breast tissue.

In collaboration with Dr. Qian Wu at Brown University Hospital in Rhode Island, Gab2 expression in five breast tumor samples was examined by immunohistochemistry using anti-Gab2. Importantly, there was strong Gab2 immunostaining in breast carcinoma cells of all the examined samples. Strong Gab2 immunostaining was found in carcinoma cells from both invasive ductal carcinoma and DCIS tumor samples. In contrast, the normal mammary epithelial ducts as well as the surrounding normal connective tissues showed weak Gab2 immunostaining, confirming that Gab2 is overexpressed specifically in tumor cells.

### Example 10

### EGF Induction of Gab2 Tyrosyl Phosphorylation in Breast Cancer Cell Line

Since overexpression of EGFR family members (ErbB2) can promote breast carcinogenesis, the overexpressed Gab2 may be involved in breast cancer by amplifying EGFR initiated signals. Gab2 tyrosyl phosphorylation and its association with signal relay molecules by Gab2 immunoprecipitation followed by immunoblotting with p85 and SHP-2 antibodies was examined. Gab2 was found to be robustly tyrosyl phosphorylated and became associated with p85 and SHP-2 upon EGF stimulation in MDA-MB-486 cells.

### Example 11

### Generation of Mammary Epithelial Cell Lines Overexpressing Gab2

Gab2 was overexpressed in two well-characterized breast epithelial cell lines to investigate the effects of Gab2 overexpression on breast cell growth. One breast epithelial cell line was the immortalized normal epithelial cell line MCF-10A. The other was the breast cancer cell line MCF-7, which expressed a low level of Gab2. MCF-7 clones that can inducibly express Gab2 using the tetracycline-off expression system were generated (Gossen, M. & Bujard, H., Proc Natl Acad Sci, USA 89, 5547-5551 (1992)). The advantage of using this expression system was that Gab2 expression level can be controlled depending on the different concentration of tetracycline present in the culture media These Gab2 overexpressing cell lines will be useful tools to study the effect of Gab2 on breast cell growth in vitro and tumor formation in mice.

### Example 12

### Effects of Gab2 Overexpression on Mammary Epithelial Cell Lines

Breast cancers mainly arise from breast epithelia, and proceed through a series of changes starting with hyperplasia with atypia and progressing to carcinoma in situ, invasive carcinoma, and eventually metastatic disease. At different stages of the carcinogenesis, breast cancer cells become highly proliferative, resistant to apoptosis, grow without maintaining polarity and/or their dependence on basement membrane, and more migratory and invasive. Mammary epithelial cell lines with stable expression of Gab2 were established, whether Gab2 overexpression enhances or promotes growth, migration/invasion, and transformation of mammary epithelial cell lines, such as immortalized mammary epithelial cells (MCF-10A) and breast cancer cell (MCF-7), in vitro can be assessed. The effects of Gab2 overexpression on tumorigenicity in nude mice can also be assessed.

### Example 13

### Effects of Gab2 Overexpression on the Immortalized Mammary Epithelial Cell Line MCF-10A

MCF-10A cell growth mainly depends on the presence of EGF in the culture medium since EGF withdrawal causes MCF-10A cells to arrest at G0/G1 (Blagosklonny, M. V. et al., Cancer Res 60, 3425-3428 (2000)) and eventually become apoptotic (Davis, J. W. et al., Carcinogenesis 21, 881-886 (2000)). First, the issue of whether Gab2 overexpression promotes MCF-10A growth in its growth medium containing EGF can be examined. An equal number of MCF-10A vector-transfected cells (V) and two clones overexpressing Gab2 can be plated in growth media. Cells can be counted every day for three days by trypan-blue exclusion. If the Gab2 overexpressing cells grow faster in this assay, the issue of whether Gab2 expression increased MCF-10A proliferation and/or decreased cell death can be determined. To quantify dead cells, cell samples can be taken every 24 hours for three days and assayed for apoptotic cells using Annexin V (which binds to the cell surface of the early apoptotic cells) reagent.

The cell cycle length of MCF-10A-V and -Gab2 cell lines can be measured to examine whether Gab2 expression increases MCF-10A cell proliferation. Appropriate cells can be pulse labeled with BrdU. Then every 3 hours for 24 hours (the typical doubling time of MCF-10A cells), an aliquot of the labeled cells can be taken and the BrdU+ cells relative to DNA content can be analyzed by Propidium Iodide (PI). Initially, essentially all BrdU+ cells should be in S phase (by PI staining). Over time, the BrdU+ cells should move progressively from S phase into G2/M, G1, and back to S phase. The length of each cell cycle phase can be determined by calculating how long it takes BrdU+ cells to traverse all the phases. A shorting of either G1, S or G2/M phase of MCF-10A-Gab2 cells compared to MCF-10A-V cells can indicate which phase of the cell cycle is affected by the Gab2 overexpression. Since EGF may induce Gab2 tyrosine phosphorylation and its association with p85 and SHP-2, but not insulin/IGF-1, Gab2 may promote MCF-10A cell growth (increase cell proliferation and/or survival) through the amplification of the EGF-initiated signal transduction.

Two assays can be applied to examine whether Gab2 overexpression causes MCF-10A growth independent of polarity and substratum in vitro. First, the ability of MCF-10A-Gab2 cells to undergo anchorage-independent growth can be tested. Equal number of MCF-10A-V and MCF-10A-Gab2 cells can be seeded in soft agar in growth media. Two weeks later, the number of colonies containing more than four cells can be scored as positive (Zelinski, D. P. et al., Cancer Res 61, 2301-2306 (2001)). Next, the issued of whether Gab2 overexpression can disrupt the formation of normal mammary tissue structures (acini) by the parental MCF-10A cells when grown on Matrigel, another property of breast cancer cells, can be tested (Weaver, V. M. et al., Semin Cancer Biol 6, 175-184 (1995)). Both MCF-10A-V and -Gab2 cells can be seeded on Matrigel coated dishes in growth media 7-10 days later, the formation of acini (a spherical structure with a single layer of cells surrounding a hollow lumen) can be examined by confocal microscopy. If the formation of disorganized cell aggregates on Matrigel and/or the appearance of positive colonies in soft agar was observed in cells overexpressing Gab2, this could suggest that Gab2 overexpression was oncogenic to normal breast epithelial cells.

Another property of aggressive breast cancer cells is that they are migratory and invasive. The effect of Gab2 expression on migration and invasion of MCF-10A cells using transwell assay can be examined. For a migration assay, cells can be seeded in the upper chamber of the transwell containing semi-permeable filters. A time course (2, 4, 6, 8 hours after incubation) of cells migrating to the bottom side of the filter can be determined. To measure invasion, a similar assay can be performed except the transwell filter can be coated with Matrigel. To measure the kinetics of cells invading through the matrix and getting onto the other side of the membrane, a longer time course of measurement can be followed.

Because in vitro transformation assays do not always predict tumorigenic potential in vivo, the issue of whether MCF-10A-Gab2 cells cause tumor formation when subcutaneously injected into nude mice can be examined. If MCF-10A-Gab2 cells are tumorigenic in nude mice, this would indicate that Gab2 overexpression may promote tumorigenesis in vivo.

Since PI-3K and SHP-2 are two major effectors of Gab2 (Gu, H. et al., Mol Cell. 2, 729-740 (1998); Gu, H. et al., Nature 412, 186-190 (2001)), the issue of whether Gab2 activated PI-3K and SHP-2 or both contribute to the potential increased growth and transformation of MCF-10A cells can be investigated. To address this question, Gab2 mutants that cannot bind PI-3K (ΔPI3K), SHP-2 (ΔSHP2), and both PI-3K and SHP-2 (ΔPI3K+SHP2) in MCF-10A cells can be expressed using the pBabe-puro retroviral vector. Pools or multiple clones (at least 3 each) of MCF-10A cells expressing similar level of Gab2 wild type (WT) and Gab2 mutants can be used for the proliferation, apoptosis, transformation, and migration/invasion assays mentioned above. One of the Gab2 ΔPI3K, ΔSHP2 and double mutants would be expected to be defective in enhancing some of the MCF-10A responses. In case all of these Gab2 mutants behave in the same way as Gab2 WT, the effects of expressing other Gab2 mutants, such as Gab2 mutant that cannot bind Crk (Crouin, C. et al., FEBS Lett 495, 148-153 (2001)), can be tested on MCF-10A growth. It has been reported that Gab1 binding to Crk correlates to the ability of Gab1 to promote transformation by oncogenic Met (Lamorte, L. et al., Oncogene 19, 5973-5981 (2000)).

### Example 14

### Gab2 Cooperation with ErbB2 on Transformation of MCF-10A Cells

It is conceivable that Gab2 overexpression only enhances MCF-10A proliferation or survival, but not transformation or tumorigenicity of MCF-10A due to the limited number of ErbB1 (EGFR) and ErbB2 on the surface of MCF-10A cells. Overexpression of Gab2 alone may not be enough to activate oncogenic cascades in MCF-10A cells.

ErbB2 overexpression in mammary gland can induce breast tumor in mice (Guy, C. T. et al. Proc Natl Acad Sci U S A 89, 10578-10582 (1992)). Interestingly, overexpressing ErbB2 cannot transform MCF-10A cells and MCF-10A-ErbB2 cells are not tumorigenic when injected into mice (Giunciuglio, D. et al., Int J Cancer 63, 815-822 (1995)). One possible explanation for this is a lack of downstream signaling component for ErbB2 oncogenic transformation in MCF-10A cells.

Therefore, the issue of whether co-overexpression of Gab2 and ErbB2 may cause transformation of MCF-10A cells can be addressed. First, MCF-10A cells overexpressing both Gab2 and ErbB2 can be generated. MCF-10A overexpressing ErbB2 (MCF-10A-ErbB2) cells (kindly provided by Sam Lee, Beth Israel Deaconess Medical Center) can be infected with pBabe-puro virus alone, or with pBabe-puro virus containing Gab2 WT, or with pBabe-puro virus containing different Gab2 mutants such as ΔPI3K and ΔSHP2. MCF-10A-ErbB2 clones overexpressing a similar level of Gab2 WT and mutants can be screened by immunoblotting with Gab2 antibodies. At least two different clones expressing each Gab2 protein can be used for the subsequent studies. Next, the issue of whether MCF-10A-ErbB2-Gab2 cells become transformed can be examined by assaying their ability to grow in soft agar, form disorganized aggregates on Matrigel (inability to form acini), and induce tumor formation when injected into nude mice. If MCF-10A-ErbB2-Gab2 cells display all of the transforming phenotypes, this would suggest that overexpression of Gab2 together with ErbB2 overexpression can contribute to breast tumor formation in vivo.

To examine how Gab2 may cooperate with ErbB2 to cause the transformation of MCF-10A cells, one can test whether MCF-10A-ErbB2 clones expressing different Gab2 mutants (as discussed) can grow in soft agar, disrupt acini formation on Matrigel, and/or form tumor in mice. It would be expected that the Gab2-ΔPI3K mutant may be defective in these transformation assays, suggesting that Gab2 activation of PI-3K was important for breast carcinogenesis, consistent with the known role of PI-3K in oncogenic transformation (Cantley, L. & Neel, B., Proc Natl Acad Sci U S A 96, p 4240-4245 (1999)). However, it cannot be ruled out that the Gab2-DSHP2 may be also defective in some of the transformation assays, considering that SHP-2 mainly functions as a positive signal transducer downstream of RTK including EGFR (Chen, B. et al., Nat Genet 24, 296-299 (2000)).

### Example 15

### Effects of Overexpressing Gab2 on the Growth/Tumorigenicity of Breast Cancer Cell MCF-7

Activation or inactivation of additional key molecules beside ErbB2 and Gab2 may be required for MCF-10A cells to become tumorigenic in mice. If overexpression of Gab2 and ErbB2 only causes transformation of MCF-10A cells in vitro (i.e., anchorage independent growth), but not tumorigenicity in mice, this would suggest that Gab2 may not be the only critical downstream target mediating the ErbB2 oncogenic signal.

To see whether overexpression of Gab2 can promote tumor formation, one can test whether overexpression of Gab2 will enhance the growth/or tumorigenicity of an already carcinogenic but relatively non-aggressive breast cancer cell MCF-7. MCF-7 cells express detectable level of ErbB2 (Cuello, M. et al., Cancer Res 61, 4892-4900 (2001)) and Gab2. It has lower invasive property (Johnson, M. D. et al., Cancer Res 53, 873-877 (1993)) and forms tumors in nude mice with longer latency (Yue, W. & Brodie, A., J Steroid Biochem Mol Biol 44, 671-673 (1993)). A MCF-7 cell line that expresses Gab2 inducibly using the tetracycline (Tet)-off system has been generated. The Tet-off system has been used widely to achieve inducible expression of a gene of interest in cultured cells (Gossen, M. & Bujard, H., Proc Natl Acad Sci, USA 89, 5547-5551 (1992)) as well as in mice (Huettner, C. S. et al., Nat. Genet 24, 57-60 (2000)) upon decreasing or withdrawing tetracycline (Tet) from culture media and drinking water.

First, the issue of whether inducing the expression of Gab2 will enhance transforming properties of MCF-7 cells in vitro can be examined. For example, soft agar and transwell invasion assay using MCF-7 Gab2 cells in the absence or presence of Tet can be performed. If overexpression of Gab2 enhances MCF-7 transformation, one would expect that the formation of larger colonies in the absence of tetracycline (Gab2 is overexpressed) compared to in the presence of tetracycline, and/or that MCF-7 cells will invade through the Matrigel faster in the absence of Tet. Next, the tumorigenic property of MCF-7-Gab2 cells can be tested by injecting the cells into nude mice that have been maintained on Tet-containing water for several days. After injection, half of the injected mice can be still kept on Tet containing water and the other half of the mice can be on Tet free water. Typically, parental MCF-7 cells form visible tumors in nude mice about two months after inoculation. If tumors are observed earlier in mice on Tet free water, this would suggest that overexpression of Gab2 promotes enhanced breast tumor formation. To confirm that the tumors with early onset are due to Gab2 overexpression, Gab2 expression in these tumors can be examined by immunoblotting with Gab2 antibodies.

### Example 16

### To Investigate Whether Gab2 is Necessary and Sufficient to Promote Breast Cancer Alone or in Cooperation with the MMTV-Neu Transgene

Although studies from Gab2 overexpression in breast epithelial cell lines in vitro suggest a role of Gab2 in breast carcinogenesis, the effect of Gab2 overexpression can be varied depending on different cell lines used in the studies. To test whether Gab2 expression is required for breast tumor formation and progress, mouse genetic approaches can be used.

### Example 17

### Overexpression of Gab2 in a Mammary Gland Using Transgenic Approach

The issue of whether Gab2 overexpression is sufficient to cause breast tumor in mice can be investigated. To address this question, transgenic mice overexpressing Gab2 specifically in the mammary gland can be generated. The transgenic construct, in which the Gab2 cDNA has two HA tags at its C-terminus, can be placed under the control of a WAP promoter, which is only active in mammary glands, to generate WAP-Gab2 transgenic mice. The generation of WAP-Gab2 transgenic mice in FvB strain lines can take about 6-8 months, during which western blot analysis can be used to check whether the founder transgenic lines specifically overexpress Gab2 in the mammary gland. Gab2 protein expressed from the transgene should run slower in SDS-PAGE because of the HA tags. By ten months, a large number of WAP4-Gab2 transgenic mice that can be used to set up big breeding colonies for tumorigenic studies can be generated.

To assess whether Gab2 overexpression in a mammary gland is sufficient to cause a breast tumor, a large breeding colony can be established by mating the WAP-Gab2 transgenic mice with the wild type FvB mice. Since it is hard predict when breast tumor will form in WAP-Gab2 mice, these mice can be kept for the duration of their normal lifespan.

Since Gab2 may cooperate with ErbB2, the issue of whether Gab2 overexpression shortens the latency of breast tumor formation in MMTV-neu transgenic mice can be studied, in case WAP-Gab2 mice alone do not develop breast tumors. Therefore, a WAP-Gab2/MMTV-neu cross can be setup at the same time as the setup of the WAP-Gab2 mice interbreeding. Since MMTV-neu mice (obtained from Jackson ImmunoResearch Laboratory, West Grove, Pa.) typically develop mammary tumors around 6-7 month (Muller, W. J. et al., Cell 54, 105-115 (1988)), the question of whether Gab2 overexpression in a mammary gland potentiates breast tumor formation induced by MMTV-neu transgene can be answered.

One possible outcome of this approach is that a WAP-Gab2 transgene alone may not cause a breast tumor, but it may potentiate breast tumor formation-induced MMTV-neu (i.e., it may significantly shorten the latency of the breast tumor onset). This would strongly imply that Gab2 overexpression in tumors is actively involved in the breast tumor progression by cooperation with one or more other oncogenes.

### Example 18

### Analyzing a Cross Between Gab2 Knockout Mice and MMTV-neu Transgenic Mice

Since published biochemical data suggest that Gab2 functions downstream of EGFR (ErbB1), the issue of whether Gab2 is required for mammary tumor induction in MMTV-neu mice can be investigated. To address this question, MMTV-neu mice can be crossed with the Gab2 knockout (−/−) mice. Gab2−/− mice are healthy, except that they have impaired allergy response. As a control, Gab2−/− mice can be crossed with MMTV-myc mice (developing mammary tumor about 8-10 months) since MMTV-neu and MMTV-myc induce mammary tumors through a different mechanism. MMTV-neu induction of breast tumors requires cyclin D1, MMTV-myc does not (Yu, Q. et al., Nature 411, 1017-1021 (2001)).

The Gab2−/− mice can be crossed with either MMTV-neu or myc mice. Gab2+/−:MMTV-neu and Gab2+/−:MMTV-myc mice will result from these initial crosses (takes about three months). Gab2+/−:MMTV-neu mice can be interbred to generate Gab2−/−:MMTV-neu and Gab2+/+:MMTV-neu mice, and Gab2+/−:MMTV-myc mice can be interbred to generate Gab2−/−:MMTV-myc and Gab2+/+:MMTV-myc mice. The progeny can be monitored for breast tumor formation in the progeny over a 14-month period, which should allow Gab2−/−:MMTV-neu or Gab2−/−:MMTV-myc mice to show delayed onset of breast tumor formation.

If Gab2−/−:MMTV-neu mice show no or decreased breast tumor development within fourteen months, and Gab2−/−:MMTV-myc mice develop tumors with the same kinetics as Gab2+/+:MMTV-myc mice, this would suggest the genetic model that MMTV-Neu cause breast cancer via a Gab2->cyclin D1 cascade. It is also possible that loss of Gab2 has no effects on the latency of breast tumor development in either MMTV-neu or MMTV-myc mice. This would suggest that breast tumor induction by the MMTV-neu or myc does not require normal endogenous levels of Gab2.

### Example 19

### Gab2 Overexpression in MCF-10A Cells in Three-Dimensional Cultures

Materials and Methods

Reagents.

MMTV-Gab2-HA was generated by inserting the Gab2 cDNA into MMTV-SV40-Bssk (from S. Muthuswamy, Cold Spring Harbor Laboratory). cDNAs encoding wild-type Gab2, and the Gab2 mutants, 3YF and DM (Gu, H. et al, Mol. Cell 2, 729-740 (1998); Gu, H. et al., Mol. Cell. Biol. 20, 7109-7120 (2000)), were subcloned into pBabepuro (Morgenstern, J. P. et al., Nucleic Acids Res. 18(12): 3587-3596 (1990)). pBabe-cyclin D1 was obtained from A. Diehl (University of Pennsylvania), pLXSNHER2 from L. Petti (Albany Medical College), psi retro-neo vectors from H. Widlund (Dana-Farber Cancer Institute), and pcDNA3-NeuNT, NYPD and YA-E constructs (Dankort, D. et al., Mol. Cell Biol. 21, 1540-1551 (2001); Dankort, D. et al., J. Biol. Chem. 276, 38921-38928 (2001)) from W. Muller (McGill University). Gab2-specific antibodies were described previously (Gu, H. et al., Mol. Cell 2, 729-740 (1998)). The following commercial antibodies were used: polyclonal Ki67-specific (Zymed), monoclonal E-cadherin-specific and GM130-specific (BD Biosciences), monoclonal human laminin-5-specific (Chemicon), polyclonal active caspase-3-specific, HER2-specific (for immunoblotting) and phosphorylated Erk-specific (Cell Signaling), polyclonal HER2-specific (Ab-4; Calbiochem), monoclonal Erk2-specific (Santa Cruz), monoclonal HA-specific clone HA.11 (Covance) and monoclonal phosphotryosine antibody 4G10 and polyclonal Gab2-specific antibodies for immunohistochemistry (Upstate Biotechnology). TO-PRO3 was obtained from Molecular Probes, UO126 was obtained from Calbiochem and growth factor-reduced Matrigel was obtained from BD Biosciences.

Retroviral Infections.

MCF10A cells (supplied by J. Brugge, Harvard Medical School) were cultured in ‘Growth Medium’ containing DMEM-F12 (Invitrogen) supplemented with 5% horse serum (Hyclone), 20 ng/ml EGF (Peprotech), 0.5 mg/ml hydrocortisone (Sigma), 100 ng/ml cholera toxin (Sigma), 10 mg/ml insulin (Sigma), 100 IU/ml penicillin and 100 mg/ml streptomycin. VSV-pseudotyped retroviruses were produced as previously described (Debnath, J. et al., Methods 30, 256-268 (2003)). Stable pools of MCF10A cells were infected with the indicated retroviruses for experiments. MCF10A cells expressing BclX_{L} were provided by J. Brugge, Harvard Medical School.

Three Dimensional Culture and Immunostaining.

Three dimensional MCF10A cultures were maintained in ‘Assay Medium’ composed of DMEM-F12 supplemented with 2% Matrigel (BD Biosciences), 2% horse serum, 5 ng/ml EGF, 0.5 μg/ml hydrocortisone, 100 ng/ml cholera toxin, 10 μl insulin, 100 IU/ml penicillin and 100 μg/ml streptomycin (Debnath, J. et al., Methods 30, 256-268 (2003)). Immunostaining was performed as previously described (Debnath, J. et al., Methods 30, 256-268 (2003)). Acini were visualized using the BioRad MRC 1024 confocal laser system and a Nikon E800 light fluorescence microscope. Proliferation was scored by quantifying the number of Ki67-positive cells in at least 150 acini.

Quantification of Cell Proliferation.

Cell proliferation was quantified by Ki67 staining at days 6 and 15. Results from four independent experiments were normalized against the average control values and are shown as means ±s.e.m.

Results

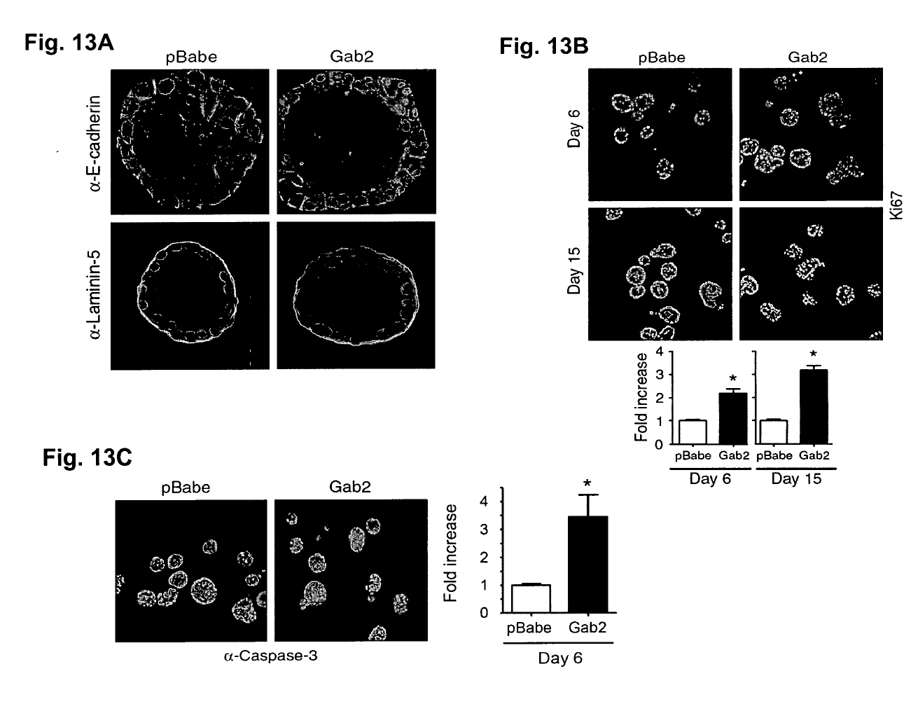

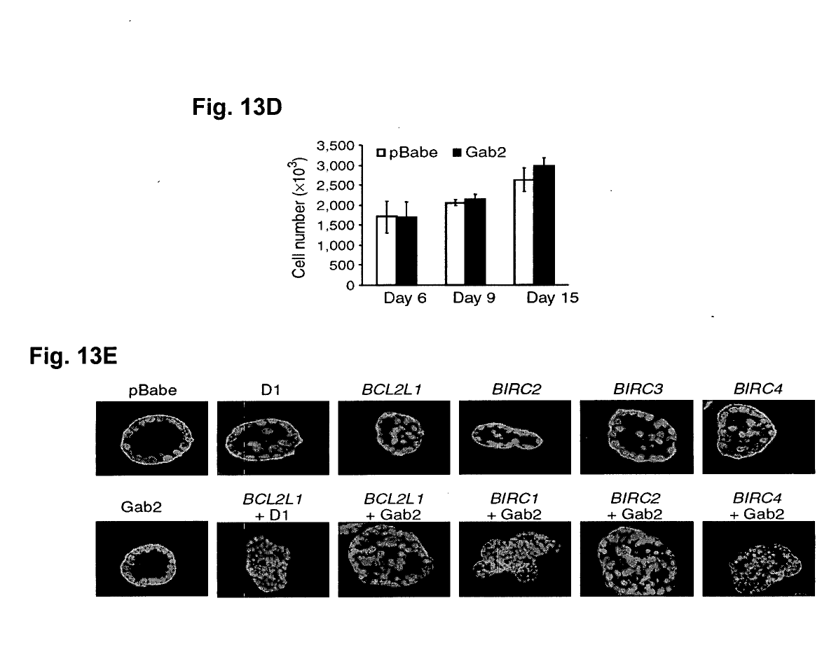

To assess the effects of Gab2 overexpression on human mammary cells, retroviral gene transduction was used to overexpress Gab2 in MCF10A cells, an immortalized, nontransformed human mammary epithelial cell line. When grown in three-dimensional culture using Matrigel, MCF10A cells resemble normal mammary epithelia, forming polarized (day 3) and growth-arrested (days 12-15) acini, which develop and maintain (days 12-20) a hollow lumen (Debnath, J. et al., Cell 111, 240 (2002)). Overexpression of Gab2 did not affect acini polarization, as assessed by immunostaining for the basal marker α6-integrin (data not shown), the apical marker GM130 (data not shown) and the junctional marker E-cadherin (FIG. 13A, top panels), or production of basement membrane, as shown by laminin-5-specific staining (FIG. 13A, bottom panels). However, compared to MCF10A cells infected with the parental control retrovirus, pBabe, Gab2-overexpressing cells showed enhanced proliferation (*P<0.0001) after 6 days of culture (FIG. 13B), at which time control cells also are proliferating, although to a lesser extent, and at 15 days of culture (FIG. 13B, bottom panels), when cell proliferation has largely ceased in control cultures. Nevertheless, Gab2-overexpressing acini retained a normal ‘gland-like’ structure, and their lumens remained hollow (FIG. 13A).

Clearing of the lumenal space in MCF10A acini results from programmed cell death and autophagy (Debnath, J. et al., Cell 111, 29-40 (2002); (Mills, K. R. et al., Proc. Natl. Acad. Sci. USA 101, 3438-3443 (2004)). Increased apoptosis in Gab2-overexpressing cells, shown by activated caspase-3 immunostaining (FIG. 13C), offset the enhanced proliferation evoked by Gab2, resulting in no net change in cell number (FIG. 13D).

Similar effects had been observed upon coexpression of cyclin D1 and antiapoptotic genes in MCF10A cells (Debnath, J. et al. Cell 111, 29-40(2002)). Notably, coexpression of Gab2 with BCL2L1, BIRC2, BIRC3 and BIRC4 (which encode BclX_{L}, c-IAP1, c-IAP2 and XIAP, respectively) led to filling of the lumenal space (FIG. 13E), suggesting that overexpression of Gab2 collaborates with antiapoptotic oncogenes to promote lumenal filling. Thus, Gab2 overexpression alone can enhance cell proliferation, while additional genetic lesions that impair proapoptotic pathways, in combination with Gab2 overexpression, allow mammary epithelial cells to become independent of extracellular matrix (ECM)-derived signals and fill the acinar lumen.

### Example 20

### Gab2 Cooperates with NeuNT to Induce an Invasive Phenotype in MCF10A Cells

Materials and Methods

Reagents.

The reagents used for these experiments are as described herein in the Materials and Methods section of Example 19.

Retroviral Infections.

Retroviral infections were performed as described herein in the Materials and Methods section of Example 19.

Three-Dimensional Culture and Immunostaining.

Three dimensional MCF10A cultures were maintained, and immunoblotting was performed, as described herein in the Materials and Methods section of Example 19.

Results

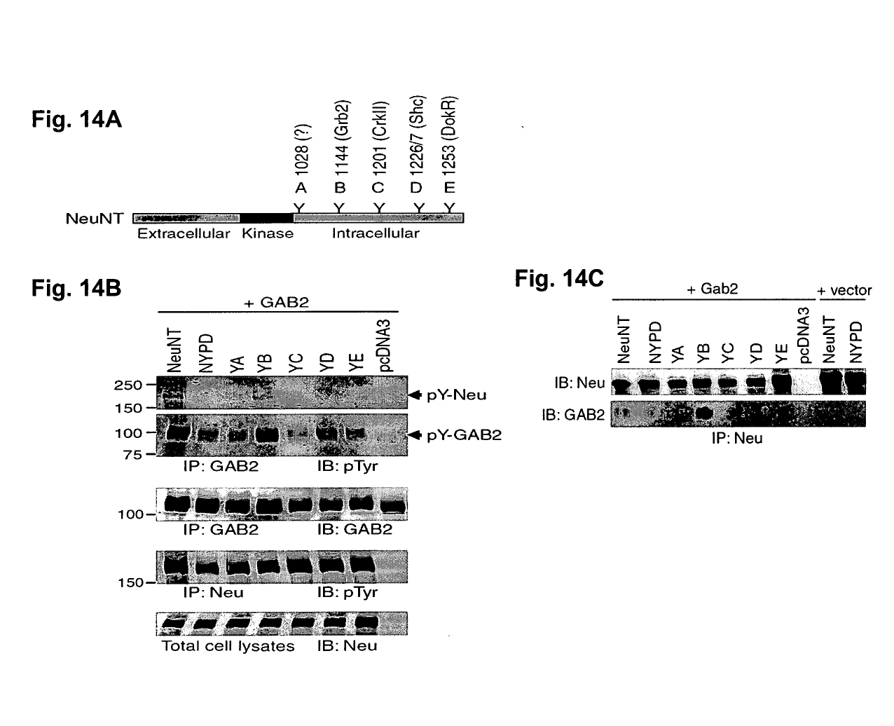

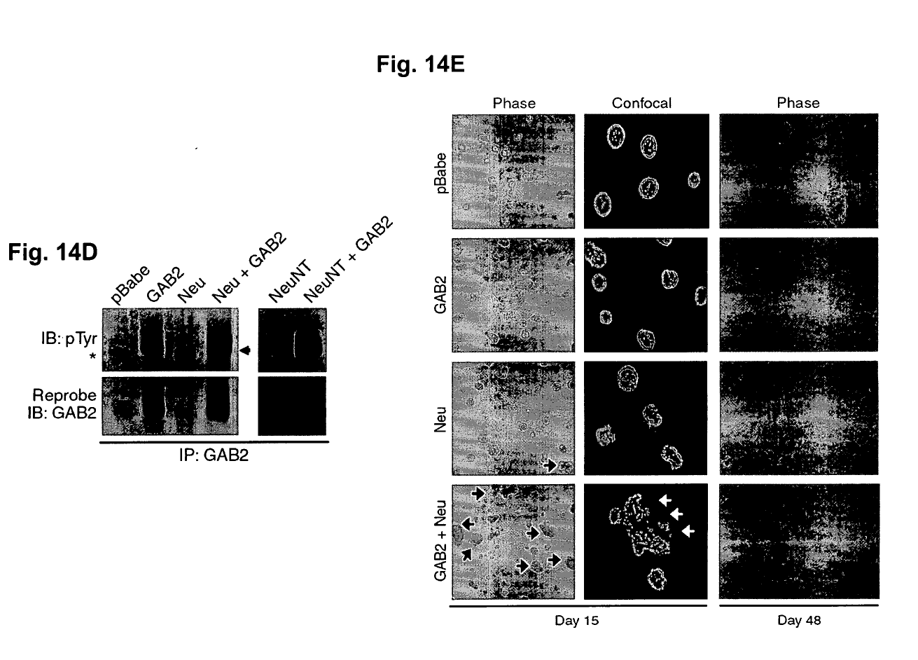

Gab2 and ErbB2 are coamplified in some breast tumors, suggesting that they might cooperate in tumorigenesis. To explore this possibility, the effects of HER2 activation on the phosphorylation and association of Gab2 was assessed. The oncogenic (activated) form of HER2 in rats (known as NeuNT) has five known sites for autophosphorylation (FIG. 14A). A panel of ‘addback’ mutants, in which single tyrosyl phosphorylation sites (labeled YA-YE) are restored to a NeuNT mutant with all of these sites mutated to phenylalanine (NYPD; FIG. 14A) (Dankort, D. et al., Mol. Cell. Biol. 21, 1540-1551 (2001), Dankort, D. et al., J. Biol. Chem. 276, 38921-38928 (2001)) were tested. 293T cells were transiently cotransfected with Gab2 and NeuNT, NYPD and the addback mutants and cell lysates were subjected to immunoprecipitation and immunoblotting analyses. Although the NeuNT mutants were expressed and tyrosyl phosphorylated to comparable extents (FIG. 14B), only NeuNT and the YB mutant coimmunoprecipitated with Gab2 and induced high levels of Gab2 tyrosyl phosphorylation (FIG. 14B, FIG. 14C). The ‘B’ site corresponds to the canonical binding site (YXNX) for the Grb2 SH2 domain (Songyang, Z. et al., Cell 72, 767-778 (1993)). Grb2 also binds, through its SH3 domains, to proline-rich regions in Gab2 (Lock, L. S. et al., J. Biol. Chem. 275, 31536-31545 (2000)). These data suggest that Gab2 is recruited to activated NeuNT in a Grb2-Gab2 complex, where it then undergoes tyrosyl phosphorylation. Although Gab2 is not expressed at detectable levels in MCF10A cells, retrovirally transduced Gab2 was tyrosyl phosphorylated in MCF10A cells, and its phosphorylation was increased further upon expression of HER2 (FIG. 14D).

Synthetic ligand-mediated dimerization of NeuNT in MCF10A cells reinitiates proliferation and induces lumenal filling, but these structures are not invasive (Muthuswamy, S. K. et al., Nat. Cell Biol. 3, 785-792 (2001)). Retrovirally-mediated overexpression of Neu, the wild-type form of NeuNT, also promoted sustained proliferation and the formation of some noninvasive, multiacinar structures (FIG. 14E; Neu panels). Compared to expression of Neu alone, coexpression of Gab2 and Neu increased the number of multiacinar structures (FIG. 14E; Gab2+ Neu panels). For example, in Neu-overexpressing cultures, 17% of structures were multiacinar at 15 d, while coexpression of Gab2 increased this to 55%. Most of these structures also showed a loss of basement membrane integrity, with individual cells streaming from the acini (FIG. 14E and data not shown). Thus, Gab2 can collaborate with Neu to cause breakdown of normal glandular architecture and promote an invasive-like phenotype in human mammary epithelial cells.

### Example 21

### Gab2 Acts Through the SHP-2-Erk Pathway

Materials and Methods

Protein Analysis.

Fifteen hours after seeding, MCF10A cells were starved for 12 h in medium containing 0.1% horse serum. Assay Medium was subsequently added for the indicated times. Three-dimensional structures were incubated with trypsin for 30 min at 37° C., and then cells were lysed in RIPA buffer (Gu, H. et al., Mol. Cell. Biol. 20, 7109-7120 (2000)). Lysates were resolved by SDS-PAGE, transferred to nylon membranes, and subjected to immunoblotting with rabbit antibody to phosphorylated Erk and mouse antibody to Erk2. After washing, the membranes were incubated with fluorescence-labeled antibodies to mouse and rabbit IgG. Images were acquired and quantified using an Infrared Imaging System (Licor Biotechnology) and Odyssey software, with phosphorylated Erk2 intensities normalized to the corresponding total Erk2 signal.

ShRNA Constructs.

A double-stranded oligonucleotide corresponding to mouse Gab2 cDNA (5′-GAAAGAGCTGCAGGATAGT-3′) was subcloned into pSuper retro-puro46 (pSR-puro) (Brummelkamp, T. R. et al., Cancer Cell 2, 243-7 (2002)) and a double-stranded oligonucleotide corresponding to human PTPN11 cDNA (5′-GAATATGGCGTCATGCGTGTT-3′) was subcloned into the psi retro-neo retroviral vector (Brummelkamp, T. R. et al., Cancer Cell 2, 243-7 (2002)). These constructs were transfected into appropriate packaging lines (Ory, D. S. et al., Proc Natl Acad Sci U S A. 93(21):11400-6 (1996 )) and transient supernatants were used for infections.

Results

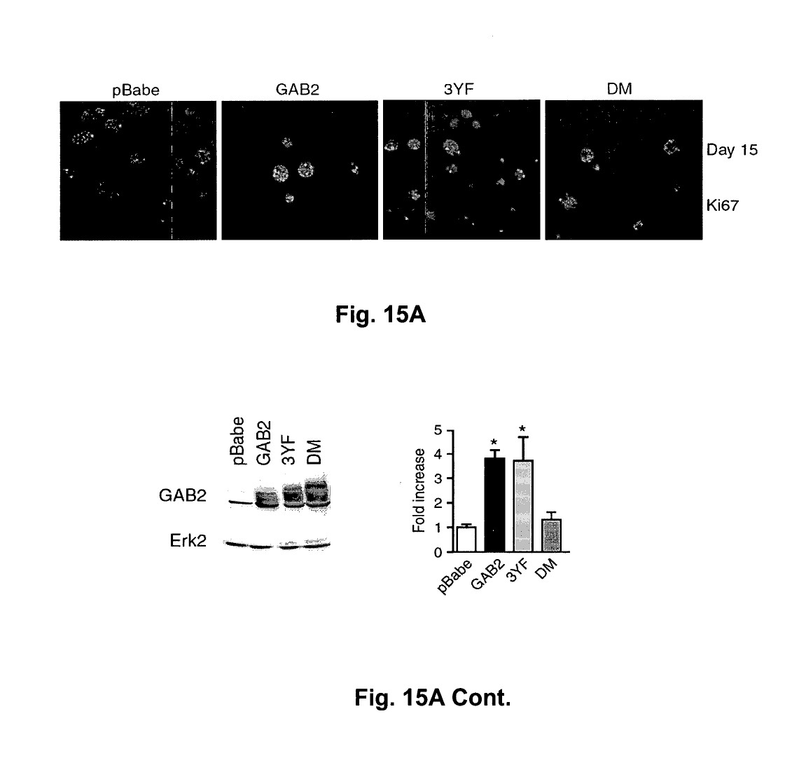

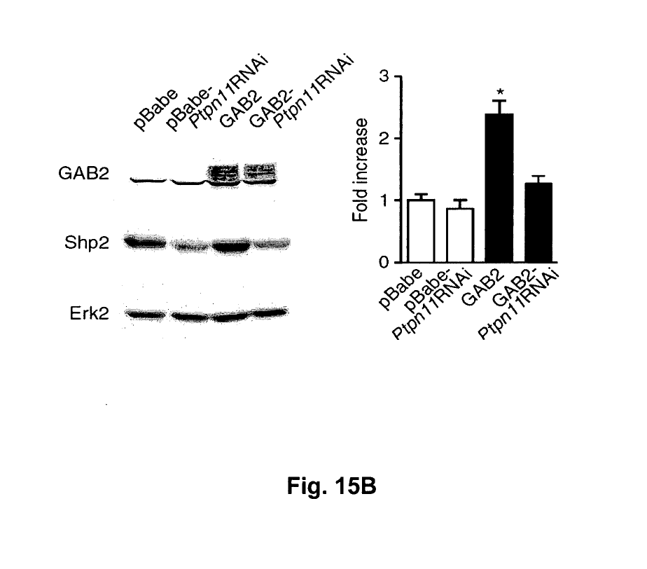

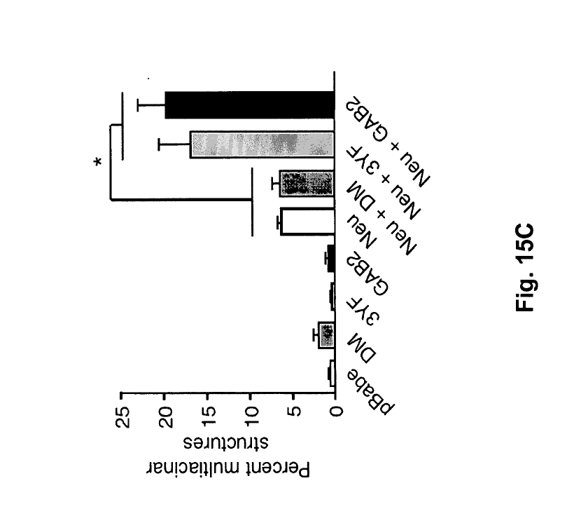

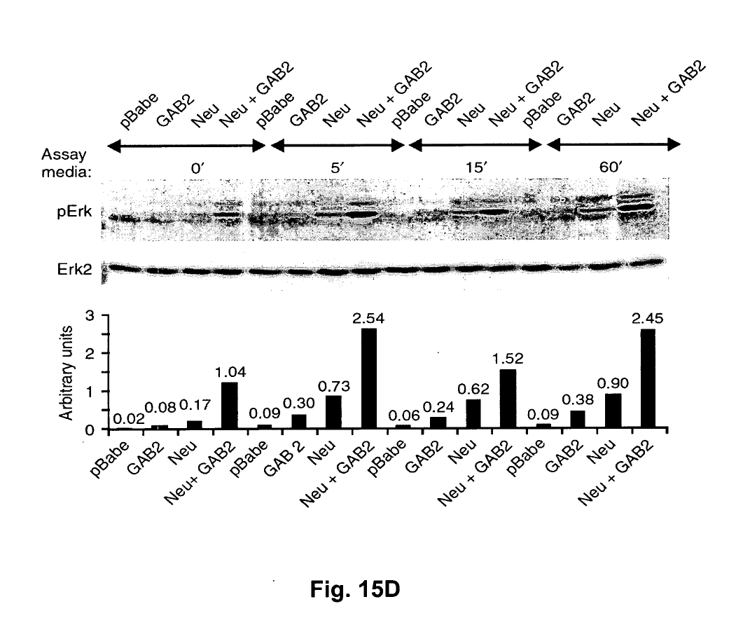

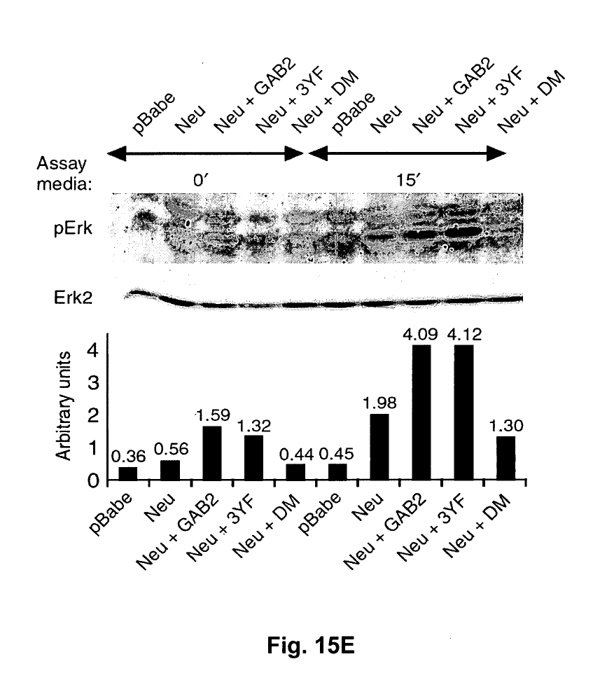

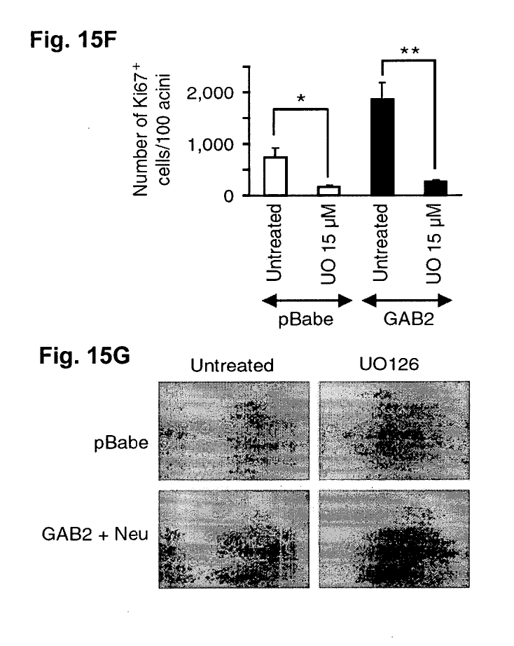

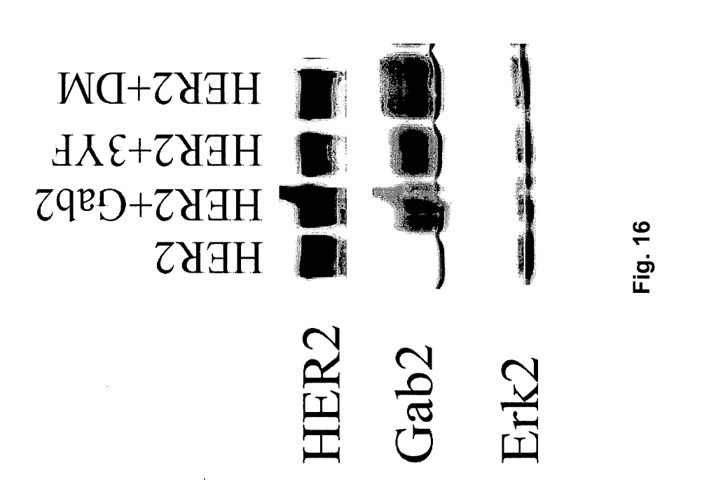

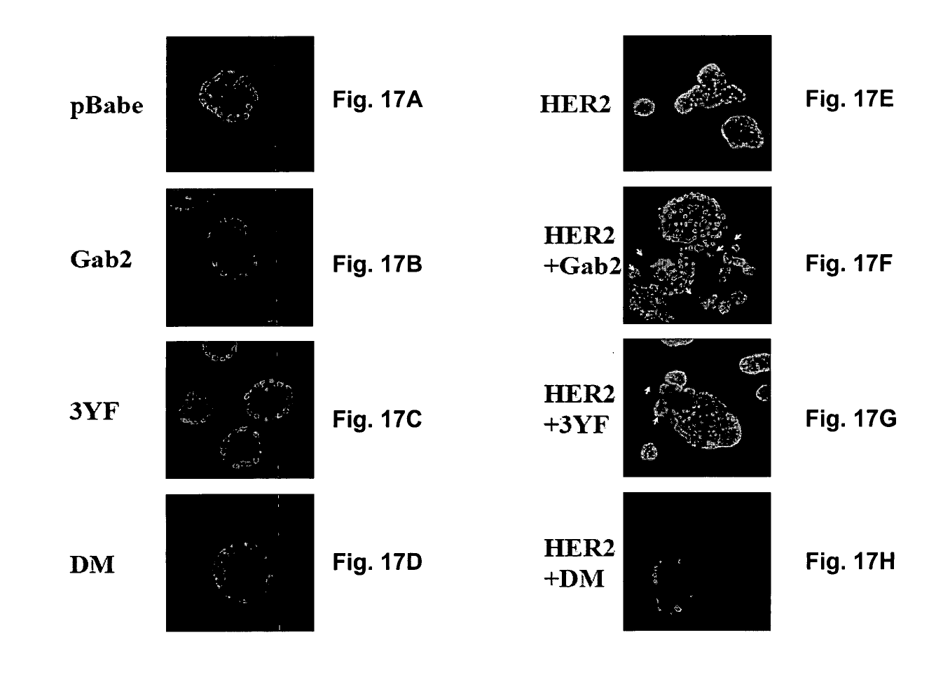

Pools of retrovirally-transduced MCF10A cells that express wild-type Gab2, a Gab2 mutant that cannot bind P13K (3YF), or a Gab2 mutant that cannot bind Shp2 (DM), respectively, were generated. Gab2-evoked hyperproliferation was impaired in DM-expressing but not 3YF-expressing cells (FIG. 15A), suggesting that Gab2 drives proliferation of MCF10A cells through Shp2. To exclude the possibility that another protein that also binds to the Shp2 binding sites on Gab2 mediates enhancement of proliferation, the effect of a retroviral vector expressing PTPN11 shRNA, which is specific for the gene encoding Shp2, was tested. Reducing Shp2 expression by 70% had little effect on proliferation of control MCF10A cells (FIG. 15B, unfilled bars). In contrast, Shp2 knockdown impaired wild-type Gab2-induced proliferation of MCF10A cells (FIG. 15B, black bars). Like wild-type Gab2, the 3YF mutant cooperated with Neu to enhance the formation of multiacinar structures (FIG. 14E). In contrast, loss of Shp2 binding, as shown with the DM mutant, abrogated the ability of Gab2 to cooperate with Neu to cause these morphologic changes (FIG. 15C, FIG. 16 and FIGS. 17A-H).

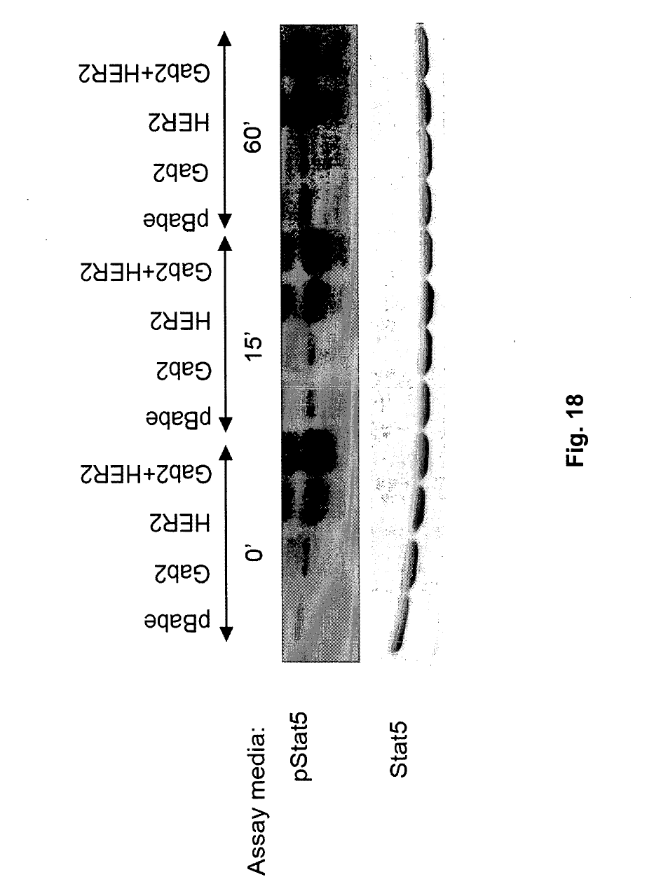

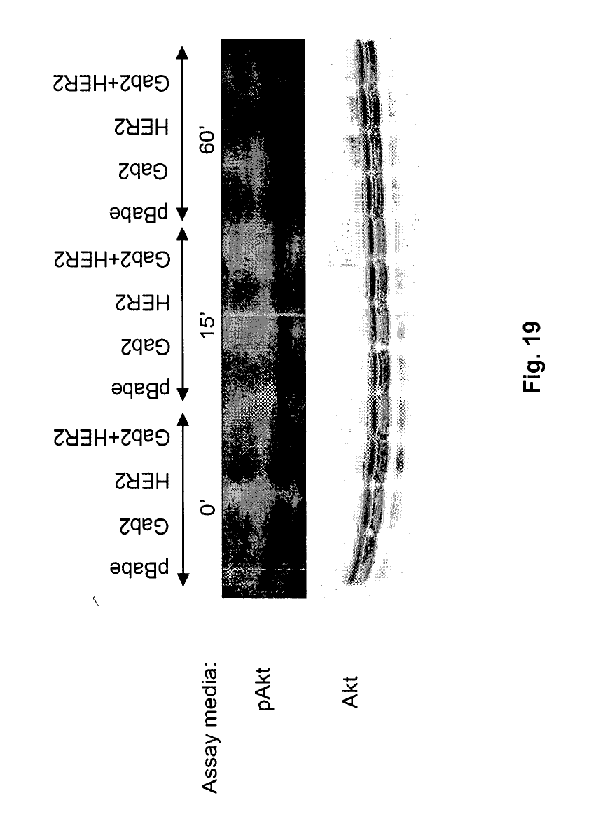

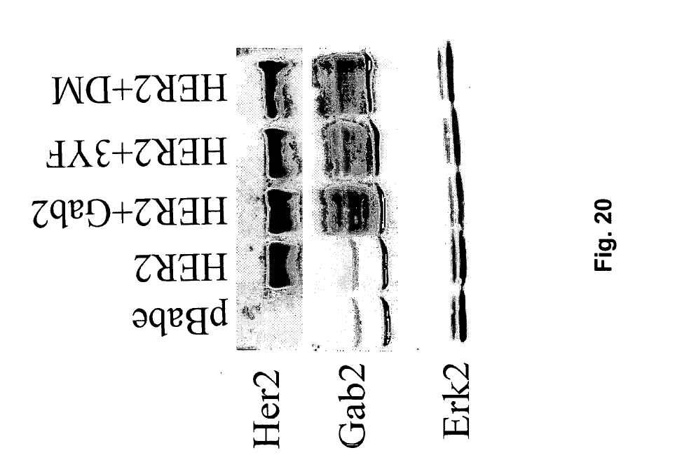

Consistent with its biological effects (FIGS. 13A-E), overexpression of Gab2 enhanced activation of Erk in MCF10A cells in three dimensional culture (FIG. 15D). Overexpression of Gab2 also enhanced Neu-evoked activation of Erk (FIG. 15D). In contrast, overexpression of Gab2 alone, or in combination with expression of Neu, had no effect on activation of Stat5 (FIG. 18) or Akt (FIG. 19). The DM mutant did not enhance activation of Erk, whereas the 3YF mutant behaved similarly to wild-type Gab2 in this assay (FIG. 15E and FIG. 20). Inhibiting the Erk pathway (using the Mek inhibitor UO126) abolished the effects of Gab2 overexpression on the proliferation of MCF10A at days 6 (data not shown) and 15 of culture (FIG. 15F), and blocked cooperation of Gab2 with Neu (FIG. 15G). These results indicate that Gab2 acts through Shp2 to enhance activation of Erk, which in turn increases proliferation of MCF10A cells, and suggest that Erk activation, together with other Neu-evoked pathways, contributes to the development of an invasive, multiacinar phenotype.

### Example 22

### Gab2 is Required for Efficient NeuNT-Evoked Tumorigenesis in Mice

Materials and Methods

Transgenic Mice.

MMTV-Gab2-HA was generated by inserting the Gab2 cDNA into MMTV-SV40-Bssk (obtained from S. Muthuswamy, Cold Spring Harbor Laboratory). MMTV-NeuNT mice (strain TG.NK) (Muller, W. J., et al., Cell 54, 105-115 (1988)) were purchased from Charles River Laboratories. Gab2^{-/-} mice (129Sv×C57B6/J) have been described previously (Gu, H. et al., Nature 412, 186-190 (2001)). Female animals from Gab2^{+/-}×NeuNT Gab2^{+/-} crosses, containing one copy of NeuNT, were kept as virgins for the entire period of the study. Mice were monitored twice weekly for tumors by palpation. An immortalized epithelial cell line was derived from an MMTV-NeuNT mammary tumor, as previously described (Gil-Henn, H. & Elson, A., J. Biol. Chem. 278, 15579-15586 (2003)). MMTV-Gab2 transgenic mice were generated by injection of a linearized MMTV-Gab2-HA construct into pronuclear zygotes obtained from FVB intercrosses, by the Beth Israel Deaconess Medical Center (BIDMC) Transgenic Facility. All animal studies were approved by the Harvard Medical Area Standing Committee on Animals.

Statistical Analysis.

Survival curves were generated using the Kaplan-Meier method, and significance was evaluated using the log rank test. Paired data were evaluated by Student t-test Comparisons between multiple groups were performed using two-way ANOVA.

Results

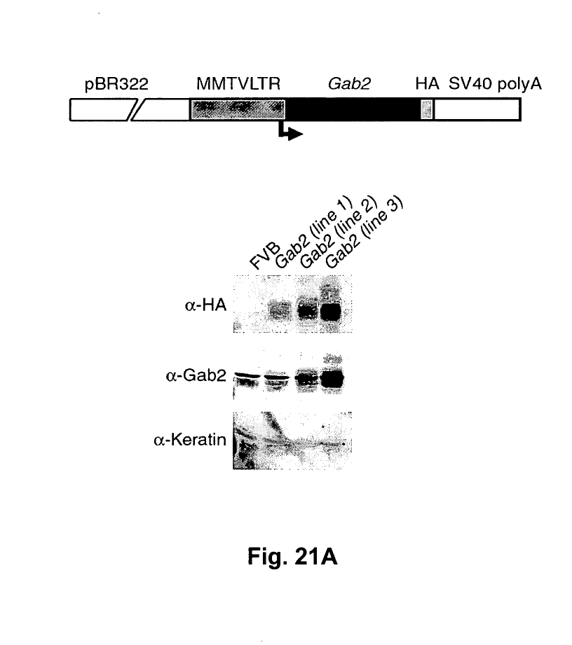

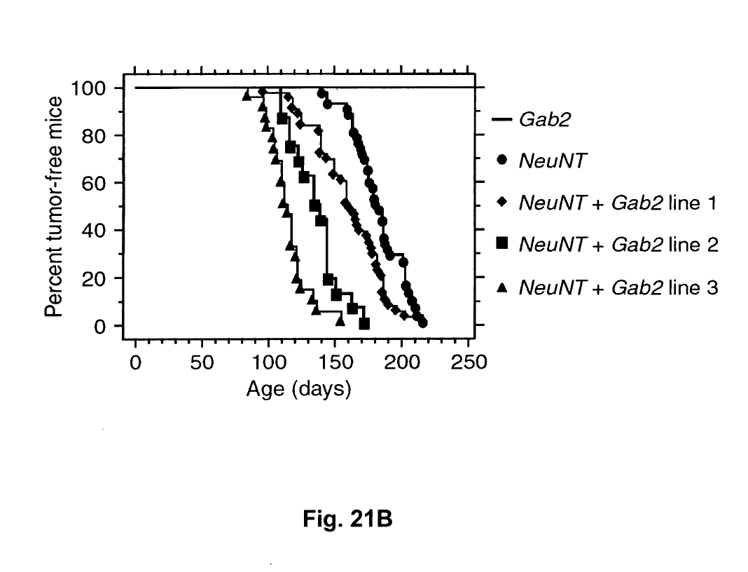

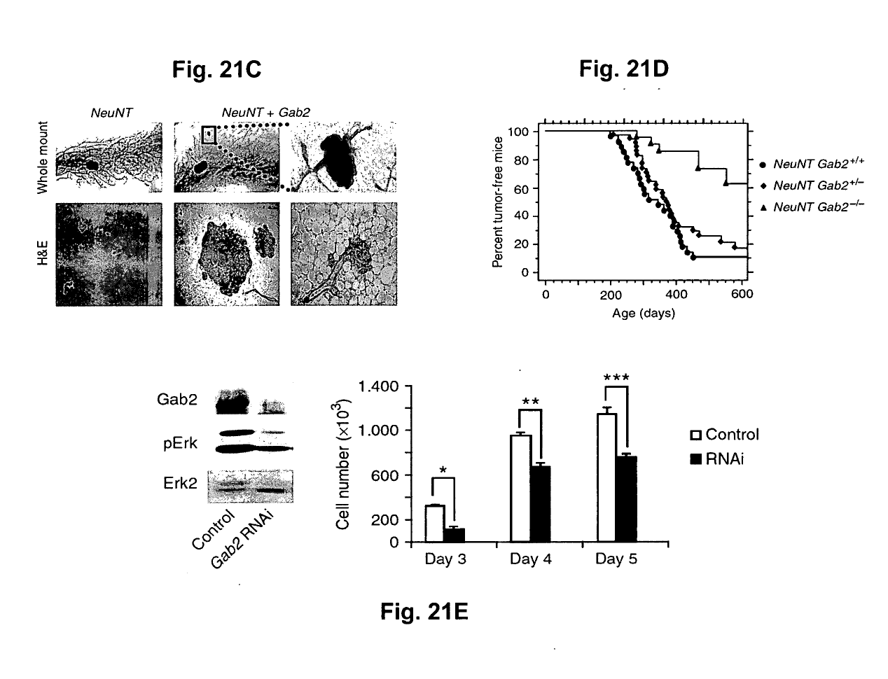

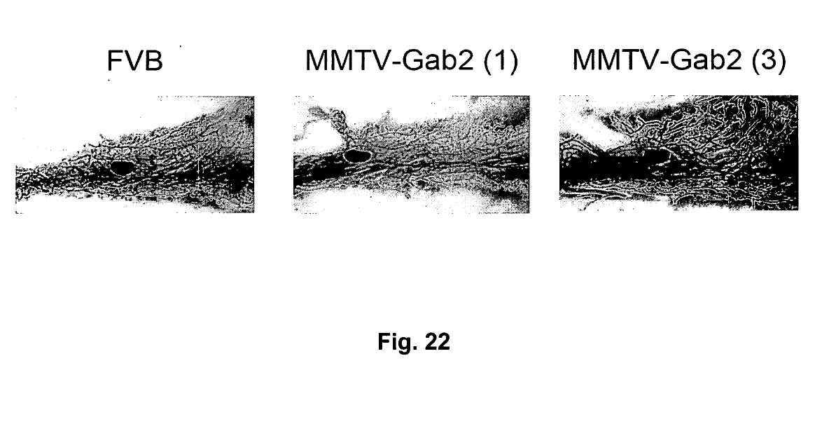

To assess the effects of Gab2 overexpression on mammary carcinogenesis in vivo, transgenic mice (FVB background) were generated with mammary-specific overexpression of HA-tagged Gab2 under the control of the mouse mammary tumor virus (MMTV) promoter (schematic in FIG. 21A). Nine founders were identified by PCR, seven of which transmitted the transgene to their progeny. Four of these lines expressed HA-Gab2, three of which expressed HA-Gab2 at levels 3 to 10-fold higher than endogenous Gab2 (FIG. 21A), which is comparable to the level of overexpression found in human breast cancer cell lines (Daly, R. J. et al., Oncogene 21, 5175-5181 (2002)). Whole mounts (FIG. 22) and H&E-stained histological sections (data not shown) of virgin mammary glands showed no obvious differences between Gab2 transgenic lines 1 and 3 and control FVB mice (n=3 for each group). Virgin littermates (n=20) from control (nontransgenic) and Gab2 transgenic lines were monitored for up to 18 months. Of these, only one mouse (from line 3) developed a mammary tumor (at 14 months). Thus, consistent with the MCF10A experiments described herein in Example 19, Gab2 overexpression alone appears to be insufficient for mammary tumorigenesis.

To assess potential Gab2-NeuNT cooperation in vivo, each of the three Gab2 transgenic lines were crossed with MMTV-NeuNT mice. As in MCF10A and transiently transfected 293T cells (FIGS. 14B-D), tyrosyl phosphorylation of Gab2 was enhanced by expression of NeuNT in bigenic mice (FIG. 14D). As expected, MMTV-NeuNT mice developed mammary tumors with a median incidence of 6 months. Overexpression of Gab2 substantially accelerated onset of tumors, and the higher the level of Gab2 expression, the faster tumors arose (FIG. 21B). Whole mounts and histological analysis showed that virgin mice expressing both Gab2 (line 3) and NeuNT developed multifocal mammary tumors as early as 12 weeks (FIG. 21C). These results indicate that overexpression of Gab2 and coexpression of NeuNT can induce mammary carcinomas in mice in a dose-dependent manner.

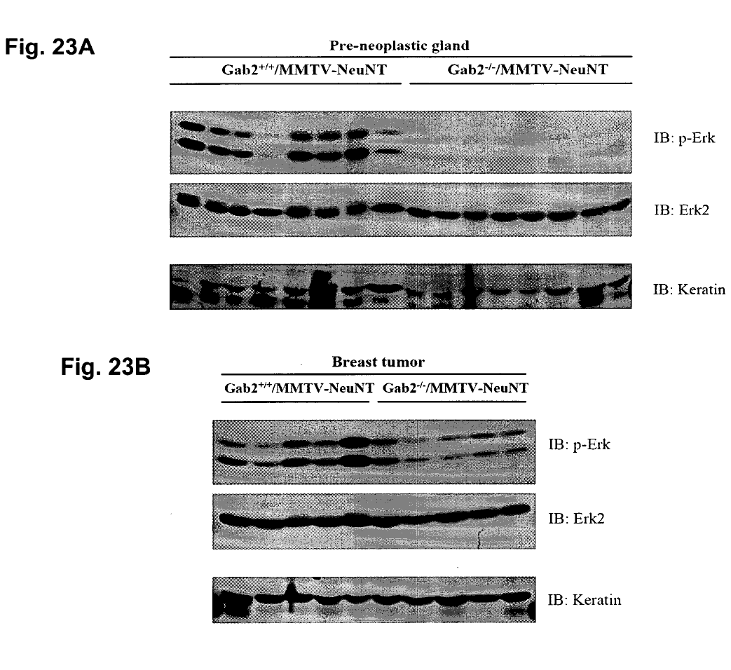

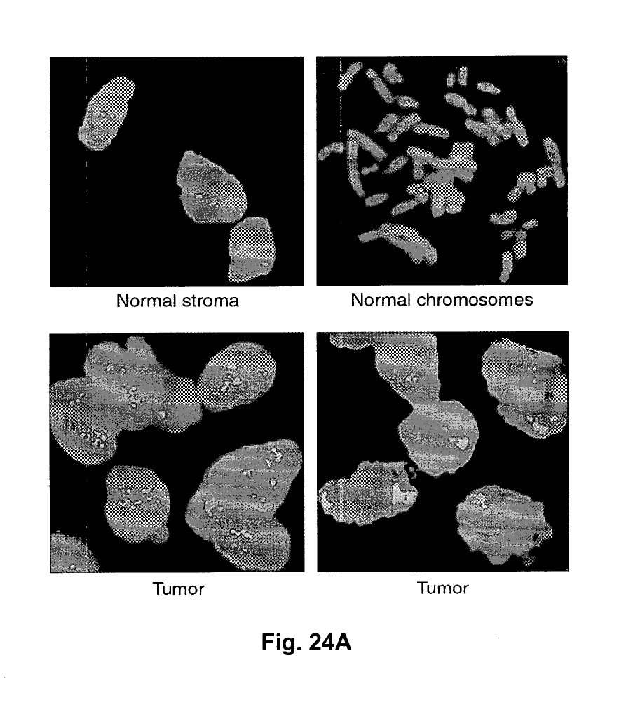

To assess whether normal levels of Gab2 are required for NeuNT-evoked tumorigenesis, Gab2 mice (129Sv×C57B6/J) were crossed with MMTV-NeuNT mice (FVB) (Muller, W. J. et al., Cell 54, 105-115 (1988)) to obtain MMTV-NeuNT Gab2^{+/+}, MMTV-NeuNT Gab2^{+/-} and MMTV-NeuNT Gab2^{-/-} mice (mixed background). As observed previously (Yu, Q., Geng, Y. & Sicinski, P., Nature 411, 1017-1021 (2001)), MMTV-NeuNT Gab2^{+/+} mice developed tumors at later times on this mixed background (FIG. 21D) than on FVB (FIG. 21B). But whereas Gab2 hemizygosity (Gab2^{+/-} genotype) had no significant effect on NeuNT-mediated tumorigenesis, MMTV-NeuNT Gab2^{-/-} mice either developed mammary tumors only after a much longer latent period than MMTV-NeuNT Gab2^{+/+} or Gab2^{+/-} mice, or did not develop tumors at all (FIG. 21D). Activation of Erk was markedly lower in mammary tissue from MMTV-NeuNT Gab2^{-/-}, compared to MMTV-NeuNT Gab2^{+/+} mice (FIG. 23A), before the development of overt tumors. Similarly, greater Erk activation was observed in tumors arising in MMTV-NeuNT Gab2^{+/+} as compared to Gab2^{-/-} littermates, although a few of tumors from this group did show significant activation of Erk (FIG. 23B). In contrast, activation of Akt was unaffected by Gab2 status in premalignant and tumor tissue (data not shown).

The effects of Gab2 levels in Neu expressing tumor cells were tested to determine whether Gab2 behaved in a cell-autonomous fashion by using a retrovirally expressed Gab2 shRNA to knock down Gab2 expression in a cell line established from an MMTV-NeuNT-induced mammary tumor. Consistent with other experiments described herein (e.g., see Example 21 and FIG. 15), knockdown of Gab2 caused decreased phosphorylation of Erk and proliferation (FIG. 21E). Therefore, signals emanating from Gab2, most notably the Erk pathway, may be required for the maintenance of mammary tumor growth induced by NeuNT.

### Example 23

### Gab2 is Amplified and Overexpressed in Breast Cancers

Materials and Methods

FISH and Immunohistochemistry.

The human Gab2 BAC clone RP11-1149C10, purchased from Children's Hospital Oakland Research Institute, was used as a probe in FISH analysis of Gab2-overexpressing human breast tumors, which were identified by microarray analyses (Matros, E. et al., Breast Cancer Res. Treat. 91, 179-186 (2005); Richardson, A. et al., Cancer Cell (in press)). FISH was performed by the Cytogenetics Core Facility at the Dana-Farber/Harvard Cancer Center. The average Gab2 gene copy number per cell was assessed by counting the total number of Gab2 hybridization spots in 100 breast tumor cells and 100 stromal cells in the same section. A ratio of Gab2 copy number in tumor/stromal cells greater than or equal to 2 was considered to be evidence of gene amplification. Gab2 immunohistochemistry was performed on frozen sections from human primary breast tumors as previously described (Daly, R. J. et al., Oncogene 21, 5175-5181 (2002)). All human breast samples were obtained as anonymous specimens from the Harvard Breast SPORE blood and tissue repository under a protocol approved by the Partners Hospital Institutional Review Board.

Histopathology and Whole-Mount Analysis.

Mammary tissue was dissected, fixed in 4% paraformaldehyde, paraffin-embedded, sectioned and stained with hematoxylin and eosin for histopathological evaluation. The protocol for whole-mount analysis of inguinal mammary glands can be found at the website mammary.nih.gov/tools/histological/wholemounts.

Results

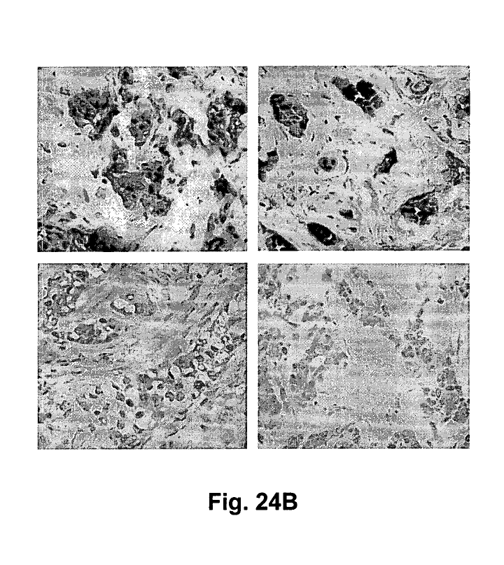

Gab2 is located on 11q13.5-14.1, a region amplified in 10-15% of human breast cancers (Ormandy, C. J. et al., Cytogenet. Cell Genet. 79, 125-131 (1997)). Fine mapping of the 11q13 region has shown four independent core regions of amplification. Core 3 harbors CCND1 (which encodes cyclin D1), overexpression of which is known to contribute to breast carcinogenesis (Ormandy, C. J. et al., Breast Cancer Res. Treat 78, 323-335 (2003)). The identity of the crucial gene(s) in core 1, where Gab2 is located, is unclear (Ormandy, C. J. et al., Breast Cancer Res. Treat. 78, 323-335 (2003)). A large cohort (142 samples) of human breast carcinomas were analyzed by microarray analysis using Affymetrix technology (Matros, E. et al., Breast Cancer Res. Treat. 91, 179-186 (2005); Richardson, A. et al., Cancer Cell (in press)). Twelve tumors showed overexpression of Gab2, defined as a>1.8-fold above the average level of expression in normal breast tissue. Of these, seven were available for fluorescence in situ hybridization (FISH) analysis (FIG. 24A), and six of these contained at least two-fold more Gab2 hybridization signals per cell compared to the adjacent normal epithelium or stroma (Table 1). Gab2 levels, assessed by immunohistochemistry, paralleled the level of Gab2 amplification (FIG. 24B). Two of the samples with Gab2 amplification also were HER2-positive by FISH (Table 1). Examination of 22 HER2-positive breast tumors from an independent cohort showed four with overexpression of Gab2, three of which also showed amplification of Gab2 by FISH (data not shown). These data suggest that amplification of Gab2 is a candidate mechanism for overexpression of Gab2 in human breast cancer.

Only some breast tumors with amplification of Gab2 amplify ERBB2. However, other RTKs, including FGFR family members, MET and IGF-1R, are overexpressed in some Gab2-amplified, Gab2-overexpressing tumors in the absence of amplification of ERBB2 (data not shown). The serine-threonine kinase PAK1 also maps within the core 1 amplicon, and is known to be amplified (Bekri, S. et al. Cytogenet. Cell Genet. 79, 125-131 (1997)) and overexpressed (Balasenthil, S. et al., J. Biol. Chem. 279, 1422-1428 (2004)) in some breast tumors. A recent study (Reyal, F. et al., Cancer Res. 65, 1376-1383 (2005)) and our own observations (data not shown) show a correlation between expression of Gab2 and PAK1 in human breast tumors, and overexpression of an activated PAK1 mutant promotes mammary hyperplasia in transgenic mice (Wang, R. A., et al., EMBO J. 21, 5437-5447 (2002)). Coamplification of 11q13 and 8p12 is often seen in breast cancer, raising the possibility that Gab2 also collaborates with gene(s) within the 8p12 region (Bautista, S. & Theillet, C., Genes Chromosom. Cancer 22, 268-277 (1998)). Our findings have potential clinical implications, as Gab2 amplification and overexpression may have prognostic significance. For example, recent microarray analyses identified Gab2 within a group of genes (‘metagene’) whose expression predicts metastasis to the lymph nodes (Huang, E. et al., Lancet 361, 1590-1596.(2003)). It will be important to design clinical studies to assess the predictive value of Gab2 amplification and overexpression in breast cancer. The results described

| | | | | GAB2 copy | | |
|---|---|---|---|---|---|---|
| | | | | number (mean) | | Ratio |
| Sample | Stage | Grade | HER2 status | Tumor (T) | Stroma (S) | T/S |
| 1 | IIIA | III | HER2⁺ | 9.5 | 1.38 | 6.9 |
| 2 | IIA | III | HER2⁺ | 16.7 | 1.51 | 11 |
| 3 | IIB | II | HER2⁻ | 14 | 1.4 | 10 |
| 4 | IIB | III | HER2⁻ | 6.4 | 1.42 | 4.5 |
| 5 | IIA | III | HER2⁻ | 3.91 | 1.63 | 2.4 |
| 6 | IIA | III | HER2⁻ | 2.98 | 1.42 | 2.1 |
| 7 | IIA | II | HER2⁻ | 1.56 | 1.59 | 0.98 |

herein also suggest that inhibitors of Mek, or upstream kinases (for example, Raf), may be particularly useful in individuals characterized by overexpression of Gab2 and/or in HER2-positive individuals having either normal or increased levels of Gab2.

Table 1 Clinical characteristics of human breast cancers with Gab2 gene amplification. Quantification of Gab2 gene copy number in breast tumors and normal adjacent stroma. FISH analysis showed two HER2⁺ samples with HER2/Centrosome 17 copy ratios of 3.1 (Sample 1) and 7.8 (Sample 2), respectively.

The relevant teachings of all publications cited herein that have not explicitly been incorporated by reference, are incorporated herein by reference in their entirety. While this invention has been particularly shown and described with references to preferred embodiments thereof, it will be understood by those skilled in the art that various changes in form and details may be made therein without departing from the scope of the invention encompassed by the appended claims.

1. An isolated nucleic acid molecule comprising:
a. Gab2 or a fragment, derivative or mutation thereof;
b. the nucleic acid sequence of SEQ ID NO. 6;
c. a sequence which encodes a polypeptide comprising amino acid sequence of SEQ ID NO. 5;
d. a nucleic acid sequence with 90% sequence identity to SEQ ID NO. 6;
e. a complementary strand of the sequence of (b), (c) or (d);
f. RNA sequences transcribed from sequences (b), (c), (d) or (e), or a fragment or mutation thereof; or
g. DNA sequences that hybridize to the sequence of (b), (d) or (e).

2-7. (canceled)

8. A transgenic non-human mammal with a genome comprising a disruption and/or an alteration of the Gab2 gene such that the mammal lacks or has altered levels of functional Gab2 protein, and wherein the mammal exhibits an altered responsiveness to cytokine, growth factor, hormone or antigen stimulation.

9-11. (canceled)

12. A method for preventing or treating a Gab2-mediated injury, comprising administering an agent which inhibits a Gab2 interaction with an associated protein.

13. The method of claim 12 wherein the Gab2 interaction with an associated protein is in response to an extracellular stimulation.

14. The method of claim 13 wherein the extracellular stimulus is a cytokine, growth factor, hormone or antigen.

15. The method of claim 12 wherein the Gab2-mediated injury is an allergic response, a neoplastic disease, or an immune disorder.

16. The method of claim 12 wherein the agent is selected from the group consisting of proteins, polypeptides, antibodies, oligonucleotides, small molecules, natural product inhibitors, mutants of Gab2, and mutants of Gab2-associated molecules.

17. The method of claim 12 wherein the agent is an oligonucleotide antisense to the nucleic acid sequence of Gab2, or antisense to a Gab2 homolog, fragment, complementary sequence, or mutant.

18. The method of claim 12 wherein the agent is a mutant Gab2, or fragment thereof, which competes with Gab2 for interaction with its associated proteins.

19. The method of claim 12 wherein tyrosyl phosphorylation of Gab2 is prevented by administration of the agent.

20. The method of claim 12 wherein expression of Gab2 is inhibited or eliminated by administration of the agent.

21. The method of claim 12 wherein the agent is administered for nasal, topical or systemic use.

22. The method of claim 12 wherein the agent is an oligonucleotide administered as an insert in a gene therapy vector.

23. The method of claim 12 wherein the associated protein is selected from the group consisting of p85, PI-3K, a protein containing a SH-2 domain, a protein containing a SH-3 domain, a protein containing a PH domain and a protein containing a WW domain.

24. The method of claim 12 wherein the agent inhibits the response of mast cells to FceRI receptor stimulation by administration to the mast cells.

25. The method of claim 12 wherein the Gab2-mediated injury is an allergic response and the agent inhibits said Gab2 interaction in response to an extracellular stimulus, thereby preventing activation of a Gab2-mediated signaling cascade.

26. The method of claim 24 wherein the response is degranulation, cytokine gene expression or anaphylaxis.

27. The method of claim 12 wherein the Gab2-mediated injury is a neoplastic disease and the agent prevents activation of a Gab2-mediated signaling cascade.

28. The method of claim 27 wherein the neoplastic disease is selected from the group consisting of leukemia, prostate cancer, ovarian cancer and breast cancer.

29. The method of claim 12 wherein the Gab2-mediated injury is breast cancer and the agent prevents activation of a Gab2-mediated signaling cascade.

30. The method of claim 12 wherein the agent is a short, double-stranded RNA molecule or a short, double-stranded RNA/DNA combination, directed against Gab2 nucleic acid sequence for the purpose of decreasing or eliminating Gab2 gene expression.

31. A method of detecting upregulation of Gab2 product in a patient with a neoplastic disorder comprising testing a sample from a patient suspected of having a neoplastic disorder with the probe of claim 7.

32. A method of identifying a drug to be administered to treat a Gab2-mediated condition in a mammal in which the condition occurs, comprising:
(a) producing a mouse that is a model of the condition;
(b) administering to the mouse a drug to be assessed for its effectiveness in treating or preventing the condition; and
(c) assessing the ability of the drug to treat or prevent the condition,
wherein if the drug reduces the extent to which the condition is present or progresses, the drug is a drug to be administered to treat the condition.

33. The method of claim 32 wherein the mouse contains a genetic mutation causing a Gab2-mediated condition.

34. The method of claim 32 wherein the Gab2-mediated condition is an allergic response, a neoplastic disease, or an immune disorder.

35. Isolated RNA that mediates RNA interference of Gab2 mRNA and/or inactivates the Gab2 gene by transcriptional silencing.

36-39. (canceled)

40. A method of mediating RNA interference of mRNA of the Gab2 gene in a cell or organism comprising:
a. introducing RNA of sufficient length which targets the mRNA of the Gab2 gene for degradation into the cell or organism; and
b. maintaining the cell or organism produced in (a) under conditions under which degradation of the mRNA occurs, thereby mediating RNA interference of the mRNA of the gene in the cell or organism.

41. (canceled)

42. The method of claim 40, further comprising, prior to step a.:
i. combining double-stranded RNA that corresponds to a sequence of the Gab2 gene with a soluble extract that mediates RNA interference, thereby producing a combination;
ii. maintaining the combination produced in (i) under conditions under which the double-stranded RNA is processed to RNA of sufficient length, thereby producing processed RNA of sufficient length; and
iii. isolating the RNA of sufficient length produced in (ii), thereby mediating RNA interference of the mRNA of the Gab2 gene in the cell or organism.

43-44. (canceled)

45. A method for treating a disease or condition associated with the presence of a Gab2 protein in an individual, comprising administering an agent to the individual, wherein the agent comprises RNA of sufficient length that targets the mRNA of the Gab2 gene for degradation.

46-48. (canceled)

49. The method of claim 12, wherein the associated protein is SHP-2.

50. The method of claim 12, wherein the associated protein is selected from the group consisting of Grb2, ERBB2/HER2/Neu and an ERBB2/HER2/Neu oncogenic mutant protein.

51. The method of claim 12, wherein the agent is a mutant Gab2 protein that is unable to bind to SHP-2 protein.

52. The method of claim 27, wherein the neoplastic disease is selected from the group consisting of leukemia, prostate cancer, ovarian cancer and breast cancer.

53. The method of claim 27, wherein the neoplastic disease is a breast cancer associated with Gab2 overexpression, a Gab2 gene amplification or a combination thereof.

54. The method of claim 52, wherein the breast cancer is associated with ERBB2/HER2/Neu overexpression or the expression of a ERBB2/HER2/Neu oncogenic mutant protein.

55. The method of claim 29, wherein the Gab2-mediated signaling cascade is an Erk signaling pathway.

56. A method for preventing or treating a disorder associated with Gab2 overexpression in a subject in need thereof, comprising administering at least one Erk signaling pathway inhibitor to the subject, whereby the disorder associated with Gab2 overexpression is prevented or treated in the subject.

57. The method of claim 56, wherein the at least one Erk signaling pathway inhibitor prevents Erk activation.

58. The method of claim 57, wherein the at least one Erk signaling pathway inhibitor comprises isolated RNA that mediates RNA interference of an mRNA for a component of an Erk-mediated signaling cascade, wherein the mRNA is selected from the group consisting of SHP-2 mRNA, Ras mRNA, Raf mRNA, Mek mRNA and Erk mRNA.

59. The method of claim 58, wherein the mRNA is SHP-2 mRNA.

60. The method of claim 57, wherein the at least one Erk signaling pathway inhibitor is a Mek inhibitor.

61. The method of claim 56, wherein the disorder associated with Gab2 overexpression is selected from the group consisting of leukemia, prostate cancer, ovarian cancer and breast cancer.

62. The method of claim 56, wherein the disorder associated with Gab2 overexpression is a breast cancer associated with a Gab2 gene amplification.

63. The method of claim 56, wherein the disorder associated with Gab2 overexpression is breast cancer associated with ERBB2/HER2/Neu overexpression or the expression of a ERBB2/HER2/Neu oncogenic mutant protein.

64. A method for preventing or treating a disorder associated with Gab2 overexpression in a subject in need thereof, comprising administering at least one agent that inhibits or eliminates Gab2 expression in one or more cells of the subject, whereby the disorder associated with Gab2 overexpression is prevented or treated in the subject.

65. The method of claim 64, wherein the disorder associated with Gab2 overexpression is breast cancer.

66. The method of claim 65, wherein the breast cancer is associated with a Gab2 gene amplification.

67. A method for diagnosing whether a subject has or is at risk for developing a disorder associated with altered Gab2 expression, comprising detecting expression of a Gab2 gene product in a sample from the subject, wherein an increase in the expression of the Gab2 gene product in the sample from the subject, relative to the expression of the Gab2 gene product in a suitable control sample, is indicative of the subject having or being at risk for developing a disorder associated with altered Gab2 expression.

68. The method of claim 67, wherein the Gab2 gene product is a Gab2 protein.

69. The method of claim 68, wherein the Gab2 protein is overexpressed.

70. The method of claim 68, wherein the expression of Gab2 protein in a sample from the subject is detected by immunohistochemical staining using a Gab2-specific antibody.

71. The method of claim 68, wherein the expression of Gab2 protein in a sample from the subject is detected by a Gab2-specific antibody using an assay selected from the group consisting of Western blot analysis, enzyme-linked immunosorbent assay (ELISA) and radioimmunoassay (RIA).

72. The method of claim 67, wherein the disorder associated with altered Gab2 expression is selected from the group consisting of leukemia, prostate cancer, ovarian cancer and breast cancer.

73. The method of claim 67, wherein the disorder associated with altered Gab2 overexpression is a breast cancer associated with a Gab2 gene amplification.

74. The method of claim 67, wherein the disorder associated with altered Gab2 overexpression is a breast cancer associated with ERBB2/HER2/Neu overexpression or the expression of a ERBB2/HER2/Neu oncogenic mutant protein.

75. A method for diagnosing whether a subject has or is at risk for developing a disorder associated with Gab2 overexpression, comprising testing one or more cells from the subject for the presence of a Gab2 gene amplification, wherein the presence of a Gab2 gene amplification in the one or more cells is indicative of the subject having or being at risk for developing a disorder associated with Gab2 overexpression.

76. The method of claim 75, wherein the presence of a Gab2 gene amplification is tested by determining a Gab2 gene copy number in one or more cells from the subject.

77. The method of claim 76, wherein a Gab2 gene copy number that is greater than 2 is indicative of the presence of a Gab2 gene amplification.

78. The method of claim 76, wherein a Gab2 gene copy number that is at least two-fold greater in cells that are affected with the disorder, relative to normal control cells from the subject, is indicative of the presence of a Gab2 gene amplification.

79. The method of claim 76, wherein the Gab2 gene copy number in the one or more cells is determined using a nucleic acid probe comprising a Gab2-specific nucleotide sequence.

80. The method of claim 79, wherein the nucleic acid probe comprising a Gab2-specific nucleotide sequence further comprises a fluorescent label.

81. The method of claim 79, wherein the nucleic acid probe comprising a Gab2-specific nucleotide sequence is BAC clone RP11-1149C10.

82. The method of claim 75, wherein the disorder associated with Gab2 overexpression is selected from the group consisting of leukemia, prostate cancer, ovarian cancer and breast cancer.

83. The method of claim 82, wherein the disorder associated with Gab2 overexpression is a breast cancer associated with ERBB2/HER2/Neu overexpression or the expression of an ERBB2/HER2/Neu oncogenic mutant protein.

84. A kit for diagnosing a disorder associated with altered Gab2 expression, comprising a Gab2-specific antibody, a nucleic acid probe that comprises a Gab2-specific nucleotide sequence, or a combination thereof.

85. The kit of claim 84, wherein the nucleic acid probe that comprises a Gab2-specific nucleotide sequence further comprises a detectable label.

86. The kit of claim 85, wherein the detectable label is a fluorophore.

87. The kit of claim 84, wherein the disorder associated with altered Gab2 expression is breast cancer.

88. The kit of claim 87, wherein the breast cancer is associated with Gab2 overexpression, a Gab2 gene amplification or a combination thereof.


SEQUENCE LISTING (56 sequences)

SEQ ID NO: 1 - protein, 6 aa, Unknown
  Murine
KAKPTP

SEQ ID NO: 2 - protein, 6 aa, Unknown
  Murine
TVIDEL

SEQ ID NO: 3 - DNA, 21 bp, Artificial Sequence
  primer sequence
ccttgacctgagaaacaacac

SEQ ID NO: 4 - protein, 6 aa, Unknown
  Murine
LDLRNN

SEQ ID NO: 5 - protein, 666 aa, Unknown
  Murine
MSGGGGDDVVCTGWLRKSPPEKKLRRYAWKKRWFILRSGRMSGDPDVLEYYKNEHSKKPLRIINLNLCEQVDAGLTFNKKELQDSFVFDIKTSERTFYLVAETEADMNKWVQSICQICGFNQAEESTDSLRNLSSASHGPRSSPAEFSSSQHLLRERKSSAPSHSSQPTLFTFEPPVSSHMQPTLSTSAPQEYLYLHQCISRRTENARSASFSQGTRQKSDTAVQKLAQSNGHCINGVGGQVHGFYSLPKPSRHNTEFKDSTYDLPRSLASHGHTKSSLTGSETDNEDVYTFKMPSNTLCRELGDLLVDNMDVPTTPLSAYQIPRTFTLDKNHNAMTVATPGDSAIAPPPRPPKPSQAETSQWGSIQQRPPISENSRSVAATIPRRNTLPAMDNSRLHRASSCETYEYPARGSGESASWSAEPPGKTAVGRSNSASSDDNYVPMNPGSSTLLAMERPGDNSQSVYIPMSPGPHHFDPLGYPSTALPIHRGPSRGSEIQPPPVNRNLKPDRKAKPTPLDLRNNTVIDELPFKSPVTKSWSRINSHTFNSSSSQYCRPISTQSITSTDSGDSEENYVPMQNPVSASPVPSGTNSPAPKKSTGSVDYLALDFQPGSPSPHRKPSTSSVTSDEKVDYVQVDKEKTQALQNTMQEWTDVRQSSEPSKGAKL

SEQ ID NO: 6 - DNA, 1965 bp, Unknown
  Murine
atgagcggcggcggcggcgacgacgtggtgtgtaccggctggctgaggaaatcgcctcccgagaagaagttgaggcgctatgcctggaagaaacgctggtttatacttcggagtggccgaatgagtggagatccagatgttctggaatactacaagaatgagcactccaagaaacccctgcggatcatcaacctgaacttgtgtgagcaggtggatgcaggcctgaccttcaacaagaaagagctgcaggatagttttgtgtttgatatcaagaccagcgagcgcacattttacctggtggctgagacagaggctgacatgaataagtgggtccagagcatctgccagatctgcggcttcaatcaggctgaagagagcacagactccctgaggaacctttcttcagccagtcatggtccccgctcttctccagctgagttcagctccagtcagcacctgctccgagaacggaagtcctcagcccttcacactctagccagcctactttattcacgtttgagccccctgtgtcaagccacatgcagcctacctgtccaccagtgcacctcaggagtatctctacttgcaccagtgcataagcagaaggacagaaaatgcaaggagtgccagcttctctcagggcacccggcagaagagtgatacagctgtgcaaaaacttgcccagagcaatggacactgtatcaacggcgtcggaggtcaagtccatggcttctatagccttcccaagccaagccgacacaatacagaattcaaagacagtacttatgatctcccacggagcctggcttcccatggccacaccaagagcagcctcacagggtctgagactgataacgaggatgtgtacaccttcaagatgcccagcaacaccctgtgtcgggaacttggagacctccttgtggacaatatggatgtcccaaccactcctctctcagcctaccagatccctagaacattcacactggacaagaaccacaatgccatgacagtggccactcctggagattcagccatagctcccccaccccggccacccaagccaagtcaggcagaaacatctcaatggggcagcattcagcaaagacctccaatcagcgaaaatagcagatctgtagctgctactatccccaggcgcaataccctccctgcaatggacaacagccgactccatcgagcttcttcctgtgagacctacgagtacccggcacgaggcagtggggaaagtgccagctggtctgctgaacctccaggaaagactgccgtaggtcgatcaaatagtgccagctctgatgacaactacgtgcccatgaacccaggttcttctaccctgctggctatggaacgaccaggggacaactcccagagtgtctacatccccatgagcccaggaccccatcactttgacccacttggctacccgccacagcccttcctattcacagaggccccagccgaggaagtgagatccagccacccccggtcaaccgaaacctcaagcctgacagaaaagcaaagccaacaccccttgacctgagaaacaacactgtcatcgatgacctgcccttcaagtcacctgtcaccaagtcttggtccaggatcaacagccacacctttaactccagttcctcccagtactgccgtcactatgtccctatgcaaaacccagtatctgcatcccctgttcccagtggcactaacagcccagctccaaagaagagtactggcagtgtggattatctcgccctggacttccagccgggctccccaagccctcaccgcaagccatccacatcatctgtcacatcagatgagaaggtagactatgtccaagtggataaagagaagacccaggccctgcagaacaccatgcaggagtggacagatgtgcgggcagtcctccgaaccttccaagggtgccaagctgtaatgaagggggccaccaag

SEQ ID NO: 7 - protein, 12 aa, Unknown
  Murine
QLEEDYYGLSKG

SEQ ID NO: 8 - protein, 7 aa, Unknown
  Murine
TQALQNT

SEQ ID NO: 9 - protein, 11 aa, Unknown
  Murine
DSTYDLPRSLA

SEQ ID NO: 10 - protein, 9 aa, Unknown
  Murine
PSRHNTEFK

SEQ ID NO: 11 - protein, 5 aa, Unknown
  Murine
VQVDK

SEQ ID NO: 12 - protein, 11 aa, Unknown
  Murine
ELQDSFVFDIK

SEQ ID NO: 13 - protein, 17 aa, Unknown
  Murine
AKPTPLDLRNNTVIDEL

SEQ ID NO: 14 - protein, 17 aa, Unknown
  Murine
SSLTGSETDNEDVYTFK

SEQ ID NO: 15 - protein, 4 aa, Unknown
  Murine
YKNE

SEQ ID NO: 16 - protein, 4 aa, Unknown
  Murine
YSLP

SEQ ID NO: 17 - protein, 4 aa, Unknown
  Murine
YDLP

SEQ ID NO: 18 - protein, 4 aa, Unknown
  Murine
YQIP

SEQ ID NO: 19 - protein, 4 aa, Unknown
  Murine
YEYP

SEQ ID NO: 20 - protein, 4 aa, Unknown
  Murine
YVPM

SEQ ID NO: 21 - protein, 4 aa, Unknown
  Murine
YIPM

SEQ ID NO: 22 - protein, 4 aa, Unknown
  Murine
YLAL

SEQ ID NO: 23 - protein, 4 aa, Unknown
  Murine
YVQV

SEQ ID NO: 24 - protein, 4 aa, Unknown
  Murine
KSPP

SEQ ID NO: 25 - protein, 118 aa, Unknown
  Murine
MSGGGGDDVVCTGWLRKSPPEKKLRRYAWKKRWFILRSGRMSGDPDVLEYYKNEHSKKPLRIINLNLCEQVDAGLTFNKKELQDSFVFDIKTSERTFYLVAETEADMNKWVQSICQIC

SEQ ID NO: 26 - protein, 115 aa, Unknown
  Murine
MSGGEVVCSGWLRKSPPEKKLKRYAWKKRWFVLRSGRLTGDPDVLEYYKNDHAKKPIRIIDLNLCQQVDAGLTFNKKEFENSYIFDINTIDRIFYLVADSEEEMNKWVRCICDIC

SEQ ID NO: 27 - protein, 112 aa, Unknown
  Murine
MDRTFYEGWLIKSPPTKRIWRARWRRRYFTLKQGEIPEQFCLEYYTDHNCRKLKGVIDLDQCEQVDCGLRLENRKQKFQYMFDIKTPKRTYYLAAETEADMRDWVNCICQVC

SEQ ID NO: 28 - protein, 4 aa, Unknown
  Murine
LEYY

SEQ ID NO: 29 - protein, 83 aa, Unknown
  Murine
PMNPNSPPRQHSSSFTEPIQEANYVPMTPGTFDFSSFGMQVPPPAHMGFRSSPKTPPRRPVPVAQCEPPPVDRNLKPDRKVKP

SEQ ID NO: 30 - protein, 4 aa, Unknown
  Murine
PMNP

SEQ ID NO: 31 - protein, 4 aa, Unknown
  Murine
PPPV

SEQ ID NO: 32 - protein, 8 aa, Unknown
  Murine
RNLKPDRK

SEQ ID NO: 33 - protein, 72 aa, Unknown
  Murine
PMNPGSSTLLAMERPGDNSQSVYIPMSPGPHHFDPLGYPSTALPIHRGPSRGSEIQPPPVNRNLKPDRKAKP

SEQ ID NO: 34 - protein, 4 aa, Unknown
  Murine
YCRP

SEQ ID NO: 35 - protein, 4 aa, Unknown
  Murine
YLYL

SEQ ID NO: 36 - protein, 9 aa, Unknown
  VARIANT 1: Xaa = Gln or Glu or Pro
XXXXXTEFK

SEQ ID NO: 37 - protein, 4 aa, Unknown
  Murine
PXXP

SEQ ID NO: 38 - protein, 4 aa, Unknown
  Murine
YVNT

SEQ ID NO: 39 - protein, 4 aa, Unknown
  Murine
YSNR

SEQ ID NO: 40 - protein, 4 aa, Unknown
  Murine
YIPI

SEQ ID NO: 41 - protein, 4 aa, Unknown
  Murine
YDTP

SEQ ID NO: 42 - protein, 4 aa, Unknown
  Murine
YDIP

SEQ ID NO: 43 - protein, 4 aa, Unknown
  Murine
YYNV

SEQ ID NO: 44 - protein, 4 aa, Unknown
  Murine
YTNA

SEQ ID NO: 45 - protein, 4 aa, Unknown
  Murine
YDFM

SEQ ID NO: 46 - protein, 4 aa, Unknown
  Murine
YFDL

SEQ ID NO: 47 - protein, 4 aa, Unknown
  Murine
YRSV

SEQ ID NO: 48 - protein, 4 aa, Unknown
  Murine
YKND

SEQ ID NO: 49 - protein, 4 aa, Unknown
  Murine
YLLL

SEQ ID NO: 50 - protein, 4 aa, Unknown
  Murine
YDSP

SEQ ID NO: 51 - protein, 4 aa, Unknown
  Murine
YNLP

SEQ ID NO: 52 - protein, 4 aa, Unknown
  Murine
YCIP

SEQ ID NO: 53 - protein, 4 aa, Unknown
  Murine
YLDL

SEQ ID NO: 54 - protein, 4 aa, Unknown
  Murine
YVVV

SEQ ID NO: 55 - DNA, 19 bp, Mus musculus
gaaagagctgcaggatagt

SEQ ID NO: 56 - DNA, 21 bp, Homo sapiens
gaatatggcgtcatgcgtgtt